(19) United States
(12) Patent Application Publication
(10) Pub. No.: US 2024/0150462 A1
(43) Pub. Date: May 9, 2024

(54) LILRB1 AND LILRB2-BINDING MOLECULES AND USES THEREFOR

(71) Applicant: UNIVERSITY HEALTH NETWORK, TORONTO (CA)

(72) Inventors: Richard BROKX, Toronto (CA); Jacqueline M. MASON, Toronto (CA); Mark Robert BRAY, Oakville (CA); Gordon S. DUNCAN, Toronto (CA)

(21) Appl. No.: 18/549,897

(22) PCT Filed: Mar. 11, 2022
(86) PCT No.: PCT/CA22/50363

§ 371 Date: Sep. 8, 2023

Related U.S. Application Data
(60) Provisional application No. 63/159,613, filed on Mar. 11, 2021.

(51) Int. Cl. C07K 16/28; A61K 45/06; A61K 39/00

(52) U.S. Cl. CPC C07K 16/2803; A61K 45/06; A61K 2039/505; C07K 2317/52; C07K 2317/565

(57) ABSTRACT

The invention provides novel anti-LILR antibodies, pharmaceutical compositions comprising such antibodies, and therapeutic methods of using such antibodies and pharmaceutical compositions for the treatment of diseases such as cancer, autoimmune disease, or allergic inflammation.

### CROSS-REFERENCE TO RELATED APPLICATIONS

This application claims priority to U.S. Provisional Patent Application No. 63/159,613, filed on Mar. 11, 2021, which is hereby incorporated by reference in its entirety.

### TECHNICAL FIELD

The present disclosure relates to antibodies that bind to one or more members of the LIRB receptor family, particularly antibodies that bind to LILRB1 and/or LILRB2, e.g., human LILRB1 and/or LILRB2 (hLILRB1 and/or LILRB2), and pharmaceutical compositions comprising such antibodies thereof. Methods of using the antibodies of the invention to detect human LILRB1 and/or LILRB2 or to modulate human LILRB1 and/or LILRB2 activity in the treatment of various diseases, including inflammatory diseases, autoimmune diseases and cancer, are also encompassed by the invention.

### BACKGROUND OF THE INVENTION

The human leukocyte immunoglobulin-like receptor [LILR, also known as immunoglobulin-like transcript (ILT)] family belongs to the superfamily of paired receptors that have the potential to transmit stimulatory or inhibitory signals according to the presence or absence of tyrosine-based signaling motifs in their cytoplasmic tail. Human LILRs consist of six stimulatory receptors (LILRA1-6) and five inhibitory receptors (LILRB1-5). LILRs are predominately expressed on myeloid and lymphoid cells and some non-immune cells, and the expression patterns are different from receptor to receptor. Polymorphism and copy-number variation contribute to diversity within humans. Receptor engagement results in intracellular phosphorylation of the tyrosine-based motifs within the receptors (LILRB) or on associated adaptor molecules (LILRA). Downstream signaling events can be mediated by phosphatases, such as SHP1, SHP2 and SHIP. In general, LILR activity can result in the upregulation or downregulation of both innate and adaptive immune functions with a range of effects on different cell types. Certain LILRs also play regulatory roles in neuronal activity and osteoclast development.

LILRB1 is broadly expressed on myeloid cells, as well as B cells and subsets of T cells and natural killer (NK) cells. LILRB2-5 are more restricted to myeloid cells and dendritic cells (DCs). Some of the ligands and signaling pathways for LILRBs have been identified. LILRB1 and LILRB2 are the best characterized receptors and both bind to classical (HLA-A, HLA-B and HLA-C) and non-classical (HLA-E, HLA-F, HLA-G and HLA-H) MHC class I or HLA class I molecules, as well as to members of the angiopoietin-like protein family. Because the immune-suppressive function of LILRBs is similar to that of the classical immune checkpoint proteins, CTLA-4 and PD-1, the interaction between LILRBs and ligands is proposed to serve as immune checkpoints. For example, engagement of LILRB1 and LILRB2 by HLA-G on cancer cells inhibits immune cell activation and generates regulatory T cells (Tregs) and suppressive antigen presenting cells (APCs), which can indirectly support tumor development. Further, the interaction of β₂-microglobulin (β2M)-associated MHC class I on cancer cells with LILRB1 on macrophages leads to loss of immune surveillance. Whether the interaction between LILRB2 and ligand also functions as a phagocytosis checkpoint is unknown. LILRBs may also represent targets for induction of transplantation tolerance to prevent allograft rejection. LILRBs (especially LILRB2 and LILRB4) are critical for induction of the tolerogenic phenotype of APCs and initiation of the T cell suppression cascade that results in immune tolerance. LILRB1 and LILRB2 can also mediate graft tolerance by binding to HLA-G. In addition to immune cells, LILRBs are expressed by cancer cells and may support malignant transformation and relapse, as well as the activity of cancer stem cells. Collectively, these findings reveal dual roles for LILRBs as immune checkpoint molecules and as tumor-sustaining factors. Development of agents useful in modulating signaling from LILRBs may be of great benefit in diseases involving dysregulation of the immune system, including cancer, inflammatory diseases and autoimmune diseases, as well as transplantation rejection.

### SUMMARY OF THE INVENTION

In one aspect, the present invention relates to novel anti-LILRB antibodies. In some embodiments, the antibodies of the presnet invention binds human LILRB1 or LILRB2.

In some embodiments, the antibody of the present invention comprises a heavy chain variable region comprising a vhCDR1, a vhCDR2 and a vhCDR3 from a heavy chain variable region comprising an amino acid sequence of SEQ ID NO:1 and a light chain variable region comprising a vlCDR1, a vlCDR2, and a vlCDR3 from a light chain variable region comprising an amino acid sequence of SEQ ID NO:5. In some embodiments, the antibody of the present invention comprises a heavy chain variable region comprising a vhCDR1, a vhCDR2 and a vhCDR3 from a heavy chain variable region comprising an amino acid sequence of SEQ ID NO:9 and a light chain variable region comprising a vlCDR1, a vlCDR2, and a vlCDR3 from a light chain variable region comprising an amino acid sequence of SEQ ID NO:13. In some embodiments, the antibody of the present invention comprises a heavy chain variable region comprising a vhCDR1, a vhCDR2 and a vhCDR3 from a heavy chain variable region comprising an amino acid sequence of SEQ ID NO:17 and a light chain variable region comprising a vlCDR1, a vlCDR2, and a vlCDR3 from a light chain variable region comprising an amino acid sequence of SEQ ID NO:21. In some embodiments, the antibody of the present invention comprises a heavy chain variable region comprising a vhCDR1, a vhCDR2 and a vhCDR3 from a heavy chain variable region comprising an amino acid sequence of SEQ ID NO:25 and a light chain variable region comprising a vlCDR1, a vlCDR2, and a vlCDR3 from a light chain variable region comprising an amino acid sequence of SEQ ID NO:29. In some embodiments, the antibody of the present invention comprises a heavy chain variable region comprising a vhCDR1, a vhCDR2 and a vhCDR3 from a heavy chain variable region comprising an amino acid sequence of SEQ ID NO:33 a light chain variable region comprising a vlCDR1, a vlCDR2, and a vlCDR3 from a light chain variable region comprising an amino acid sequence of SEQ ID NO:37. In some embodiments, the antibody of the present invention comprises a heavy chain variable region comprising a vhCDR1, a vhCDR2 and a vhCDR3 from a heavy chain variable region comprising an amino acid sequence of SEQ ID NO:41 and a light chain variable region comprising a vlCDR1, a vlCDR2, and a vlCDR3 from a light chain variable region comprising an amino acid sequence of SEQ ID NO:45. In some embodiments, the antibody of the present invention comprises a heavy chain variable region comprising a vhCDR1, a vhCDR2 and a vhCDR3 from a heavy chain variable region comprising an amino acid sequence of SEQ ID NO:49 and a light chain variable region comprising a vlCDR1, a vlCDR2, and a vlCDR3 from a light chain variable region comprising an amino acid sequence of SEQ ID NO:53. In some embodiments, the antibody of the present invention comprises a heavy chain variable region comprising a vhCDR1, a vhCDR2 and a vhCDR3 from a heavy chain variable region comprising an amino acid sequence of SEQ ID NO:57 and a light chain variable region comprising a vlCDR1, a vlCDR2, and a vlCDR3 from a light chain variable region comprising an amino acid sequence of SEQ ID NO:61. In some embodiments, the antibody of the present invention comprises a heavy chain variable region comprising a vhCDR1, a vhCDR2 and a vhCDR3 from a heavy chain variable region comprising an amino acid sequence of SEQ ID NO:65 and a light chain variable region comprising a vlCDR1, a vlCDR2, and a vlCDR3 from a light chain variable region comprising an amino acid sequence of SEQ ID NO:69. In some embodiments, the antibody of the present invention comprises a heavy chain variable region comprising a vhCDR1, a vhCDR2 and a vhCDR3 from a heavy chain variable region comprising an amino acid sequence of SEQ ID NO:73 and a light chain variable region comprising a vlCDR1, a vlCDR2, and a vlCDR3 from a light chain variable region comprising an amino acid sequence of SEQ ID NO:77. In some embodiments, the antibody of the present invention comprises a heavy chain variable region comprising a vhCDR1, a vhCDR2 and a vhCDR3 from a heavy chain variable region comprising an amino acid sequence of SEQ ID NO:81 and a light chain variable region comprising a vlCDR1, a vlCDR2, and a vlCDR3 from a light chain variable region comprising an amino acid sequence of SEQ ID NO:85. In some embodiments, the antibody of the present invention comprises a heavy chain variable region comprising a vhCDR1, a vhCDR2 and a vhCDR3 from a heavy chain variable region comprising an amino acid sequence of SEQ ID NO:89 and a light chain variable region comprising a vlCDR1, a vlCDR2, and a vlCDR3 from a light chain variable region comprising an amino acid sequence of SEQ ID NO:93. In some embodiments, the antibody of the present invention comprises a heavy chain variable region comprising a vhCDR1, a vhCDR2 and a vhCDR3 from a heavy chain variable region comprising an amino acid sequence of SEQ ID NO:97 and a light chain variable region comprising a vlCDR1, a vlCDR2, and a vlCDR3 from a light chain variable region comprising an amino acid sequence of SEQ ID NO:101. In some embodiments, the antibody of the present invention comprises a heavy chain variable region comprising a vhCDR1, a vhCDR2 and a vhCDR3 from a heavy chain variable region comprising an amino acid sequence of SEQ ID NO:105 and a light chain variable region comprising a vlCDR1, a vlCDR2, and a vlCDR3 from a light chain variable region comprising an amino acid sequence of SEQ ID NO:109. In some embodiments, the antibody of the present invention comprises a heavy chain variable region comprising a vhCDR1, a vhCDR2 and a vhCDR3 from a heavy chain variable region comprising an amino acid sequence of SEQ ID NO: 113 and a light chain variable region comprising a vlCDR1, a vlCDR2, and a vlCDR3 from a light chain variable region comprising an amino acid sequence of SEQ ID NO:117. In some embodiments, the antibody of the present invention comprises a heavy chain variable region comprising a vhCDR1, a vhCDR2 and a vhCDR3 from a heavy chain variable region comprising an amino acid sequence of SEQ ID NO:121 and a light chain variable region comprising a vlCDR1, a vlCDR2, and a vlCDR3 from a light chain variable region comprising an amino acid sequence of SEQ ID NO:125. In some embodiments, the antibody of the present invention comprises a heavy chain variable region comprising a vhCDR1, a vhCDR2 and a vhCDR3 from a heavy chain variable region comprising an amino acid sequence of SEQ ID NO: 129 and a light chain variable region comprising a vlCDR1, a vlCDR2, and a vlCDR3 from a light chain variable region comprising an amino acid sequence of SEQ ID NO:133. In some embodiments, the antibody of the present invention comprises a heavy chain variable region comprising a vhCDR1, a vhCDR2 and a vhCDR3 from a heavy chain variable region comprising an amino acid sequence of SEQ ID NO:137 and a light chain variable region comprising a vlCDR1, a vlCDR2, and a vlCDR3 from a light chain variable region comprising an amino acid sequence of SEQ ID NO:141. In some embodiments, the antibody of the present invention comprises a heavy chain variable region comprising a vhCDR1, a vhCDR2 and a vhCDR3 from a heavy chain variable region comprising an amino acid sequence of SEQ ID NO: 145 and a light chain variable region comprising a vlCDR1, a vlCDR2, and a vlCDR3 from a light chain variable region comprising an amino acid sequence of SEQ ID NO:149. In some embodiments, the antibody of the present invention comprises a heavy chain variable region comprising a vhCDR1, a vhCDR2 and a vhCDR3 from a heavy chain variable region comprising an amino acid sequence of SEQ ID NO:153 and a light chain variable region comprising a vlCDR1, a vlCDR2, and a vlCDR3 from a light chain variable region comprising an amino acid sequence of SEQ ID NO:157. In some embodiments, the antibody of the present invention comprises a heavy chain variable region comprising a vhCDR1, a vhCDR2 and a vhCDR3 from a heavy chain variable region comprising an amino acid sequence of SEQ ID NO: 161 and a light chain variable region comprising a vlCDR1, a vlCDR2, and a vlCDR3 from a light chain variable region comprising an amino acid sequence of SEQ ID NO:165. In some embodiments, the antibody of the present invention comprises a heavy chain variable region comprising a vhCDR1, a vhCDR2 and a vhCDR3 from a heavy chain variable region comprising an amino acid sequence of SEQ ID NO:169 and a light chain variable region comprising a vlCDR1, a vlCDR2, and a vlCDR3 from a light chain variable region comprising an amino acid sequence of SEQ ID NO:173. In some embodiments, the antibody of the present invention comprises a heavy chain variable region comprising a vhCDR1, a vhCDR2 and a vhCDR3 from a heavy chain variable region comprising an amino acid sequence of SEQ ID NO: 177 and a light chain variable region comprising a vlCDR1, a vlCDR2, and a vlCDR3 from a light chain variable region comprising an amino acid sequence of SEQ ID NO:181.

In some embodiments, the antibody of the present invention comprises a heavy chain variable region comprising a vhCDR1, a vhCDR2 and a vhCDR3 from a heavy chain variable region comprising an amino acid sequence of SEQ ID NO:185 and a light chain variable region comprising a vlCDR1, a vlCDR2, and a vlCDR3 from a light chain variable region comprising an amino acid sequence of SEQ ID NO:189. In some embodiments, the antibody of the present invention comprises a heavy chain variable region comprising a vhCDR1, a vhCDR2 and a vhCDR3 from a heavy chain variable region comprising an amino acid sequence of SEQ ID NO: 193 and a light chain variable region comprising a vlCDR1, a vlCDR2, and a vlCDR3 from a light chain variable region comprising an amino acid sequence of SEQ ID NO:197. In some embodiments, the antibody of the present invention comprises a heavy chain variable region comprising a vhCDR1, a vhCDR2 and a vhCDR3 from a heavy chain variable region comprising an amino acid sequence of SEQ ID NO:201 and a light chain variable region comprising a vlCDR1, a vlCDR2, and a vlCDR3 from a light chain variable region comprising an amino acid sequence of SEQ ID NO:204. In some embodiments, the antibody of the present invention comprises a heavy chain variable region comprising a vhCDR1, a vhCDR2 and a vhCDR3 from a heavy chain variable region comprising an amino acid sequence of SEQ ID NO:208 and a light chain variable region comprising a vlCDR1, a vlCDR2, and a vlCDR3 from a light chain variable region comprising an amino acid sequence of SEQ ID NO:212.

In some embodiments, the antibody of the present invention comprises a vhCDR1 comprising SEQ ID NO:2, a vhCDR2 comprising SEQ ID NO:3, a vhCDR3 comprising SEQ ID NO:4, a vlCDR1 comprising SEQ ID NO:6, a vlCDR2 comprising SEQ ID NO:7, and a vlCDR3 comprising SEQ ID NO:8. In some embodiments, the antibody of the present invention comprises a vhCDR1 comprising SEQ ID NO:10, a vhCDR2 comprising SEQ ID NO:11, a vhCDR3 comprising SEQ ID NO:12, a vlCDR1 comprising SEQ ID NO:14, a vlCDR2 comprising SEQ ID NO:15, and a vlCDR3 comprising SEQ ID NO:16. In some embodiments, the antibody of the present invention comprises a vhCDR1 comprising SEQ ID NO:18, a vhCDR2 comprising SEQ ID NO:19, a vhCDR3 comprising SEQ ID NO:20, a vlCDR1 comprising SEQ ID NO:22, a vlCDR2 comprising SEQ ID NO:23, and a vlCDR3 comprising SEQ ID NO:24. In some embodiments, the antibody of the present invention comprises a vhCDR1 comprising SEQ ID NO:26, a vhCDR2 comprising SEQ ID NO:27, a vhCDR3 comprising SEQ ID NO:28, a vlCDR1 comprising SEQ ID NO:30, a vlCDR2 comprising SEQ ID NO:31, and a vlCDR3 comprising SEQ ID NO:32. In some embodiments, the antibody of the present invention comprises a vhCDR1 comprising SEQ ID NO:34, a vhCDR2 comprising SEQ ID NO:35, a vhCDR3 comprising SEQ ID NO:36, a vlCDR1 comprising SEQ ID NO:38, a vlCDR2 comprising SEQ ID NO:39, and a vlCDR3 comprising SEQ ID NO:40. In some embodiments, the antibody of the present invention comprises a vhCDR1 comprising SEQ ID NO:42, a vhCDR2 comprising SEQ ID NO:43, a vhCDR3 comprising SEQ ID NO:44, a vlCDR1 comprising SEQ ID NO:46, a vlCDR2 comprising SEQ ID NO:47, and a vlCDR3 comprising SEQ ID NO:48. In some embodiments, the antibody of the present invention comprises a vhCDR1 comprising SEQ ID NO:50, a vhCDR2 comprising SEQ ID NO:51, a vhCDR3 comprising SEQ ID NO:52, a vlCDR1 comprising SEQ ID NO:54, a vlCDR2 comprising SEQ ID NO:55, and a vlCDR3 comprising SEQ ID NO:56. In some embodiments, the antibody of the present invention comprises a vhCDR1 comprising SEQ ID NO:58, a vhCDR2 comprising SEQ ID NO:59, a vhCDR3 comprising SEQ ID NO:60, a vlCDR1 comprising SEQ ID NO:62, a vlCDR2 comprising SEQ ID NO:63, and a vlCDR3 comprising SEQ ID NO:64. In some embodiments, the antibody of the present invention comprises a vhCDR1 comprising SEQ ID NO:66, a vhCDR2 comprising SEQ ID NO:67, a vhCDR3 comprising SEQ ID NO:68, a vlCDR1 comprising SEQ ID NO:70, a vlCDR2 comprising SEQ ID NO:71, and a vlCDR3 comprising SEQ ID NO:72. In some embodiments, the antibody of the present invention comprises a vhCDR1 comprising SEQ ID NO:74, a vhCDR2 comprising SEQ ID NO:75, a vhCDR3 comprising SEQ ID NO:76, a vlCDR1 comprising SEQ ID NO:78, a vlCDR2 comprising SEQ ID NO:79, and a vlCDR3 comprising SEQ ID NO:80. In some embodiments, the antibody of the present invention comprises a vhCDR1 comprising SEQ ID NO:82, a vhCDR2 comprising SEQ ID NO:83, a vhCDR3 comprising SEQ ID NO:84, a vlCDR1 comprising SEQ ID NO:86, a vlCDR2 comprising SEQ ID NO:87, and a vlCDR3 comprising SEQ ID NO:88. In some embodiments, the antibody of the present invention comprises a vhCDR1 comprising SEQ ID NO:90, a vhCDR2 comprising SEQ ID NO:91, a vhCDR3 comprising SEQ ID NO:92, a vlCDR1 comprising SEQ ID NO:94, a vlCDR2 comprising SEQ ID NO:95, and a vlCDR3 comprising SEQ ID NO:96. In some embodiments, the antibody of the present invention comprises a vhCDR1 comprising SEQ ID NO:98, a vhCDR2 comprising SEQ ID NO:99, a vhCDR3 comprising SEQ ID NO: 100, a vlCDR1 comprising SEQ ID NO:102, a vlCDR2 comprising SEQ ID NO:103, and a vlCDR3 comprising SEQ ID NO:104. In some embodiments, the antibody of the present invention comprises a vhCDR1 comprising SEQ ID NO:106, a vhCDR2 comprising SEQ ID NO:107, a vhCDR3 comprising SEQ ID NO:108, a vlCDR1 comprising SEQ ID NO:110, a vlCDR2 comprising SEQ ID NO:111, and a vlCDR3 comprising SEQ ID NO:112. In some embodiments, the antibody of the present invention comprises a vhCDR1 comprising SEQ ID NO:114, a vhCDR2 comprising SEQ ID NO:115, a vhCDR3 comprising SEQ ID NO:116, a vlCDR1 comprising SEQ ID NO:118, a vlCDR2 comprising SEQ ID NO:119, and a vlCDR3 comprising SEQ ID NO: 120. In some embodiments, the antibody of the present invention comprises a vhCDR1 comprising SEQ ID NO:122, a vhCDR2 comprising SEQ ID NO:123, a vhCDR3 comprising SEQ ID NO: 124, a vlCDR1 comprising SEQ ID NO:126, a vlCDR2 comprising SEQ ID NO: 127, and a vlCDR3 comprising SEQ ID NO:128. In some embodiments, the antibody of the present invention comprises a vhCDR1 comprising SEQ ID NO:130, a vhCDR2 comprising SEQ ID NO:131, a vhCDR3 comprising SEQ ID NO:132, a vlCDR1 comprising SEQ ID NO: 134, a vlCDR2 comprising SEQ ID NO:135, and a vlCDR3 comprising SEQ ID NO: 136. In some embodiments, the antibody of the present invention comprises a vhCDR1 comprising SEQ ID NO:138, a vhCDR2 comprising SEQ ID NO:139, a vhCDR3 comprising SEQ ID NO:140, a vlCDR1 comprising SEQ ID NO: 142, a vlCDR2 comprising SEQ ID NO:143, and a vlCDR3 comprising SEQ ID NO:144. In some embodiments, the antibody of the present invention comprises a vhCDR1 comprising SEQ ID NO:146, a vhCDR2 comprising SEQ ID NO:147, a vhCDR3 comprising SEQ ID NO:148, a vlCDR1 comprising SEQ ID NO:150, a vlCDR2 comprising SEQ ID NO:151, and a vlCDR3 comprising SEQ ID NO: 152. In some embodiments, the antibody of the present invention comprises a vhCDR1 comprising SEQ ID NO: 154, a vhCDR2 comprising SEQ ID NO:155, a vhCDR3 comprising SEQ ID NO:156, a vlCDR1 comprising SEQ ID NO: 158, a vlCDR2 comprising SEQ ID NO:159, and a vlCDR3 comprising SEQ ID NO:160. In some embodiments, the antibody of the present invention comprises a vhCDR1 comprising SEQ ID NO:162, a vhCDR2 comprising SEQ ID NO:163, a vhCDR3 comprising SEQ ID NO:164, a vlCDR1 comprising SEQ ID NO:166, a vlCDR2 comprising SEQ ID NO:167, and a vlCDR3 comprising SEQ ID NO:168. In some embodiments, the antibody of the present invention comprises a vhCDR1 comprising SEQ ID NO: 170, a vhCDR2 comprising SEQ ID NO:171, a vhCDR3 comprising SEQ ID NO:172, a vlCDR1 comprising SEQ ID NO: 174, a vlCDR2 comprising SEQ ID NO:175, and a vlCDR3 comprising SEQ ID NO:176. In some embodiments, the antibody of the present invention comprises a vhCDR1 comprising SEQ ID NO:178, a vhCDR2 comprising SEQ ID NO:179, a vhCDR3 comprising SEQ ID NO:180, a vlCDR1 comprising SEQ ID NO:182, a vlCDR2 comprising SEQ ID NO:183, and a vlCDR3 comprising SEQ ID NO:184. In some embodiments, the antibody of the present invention comprises a vhCDR1 comprising SEQ ID NO: 186, a vhCDR2 comprising SEQ ID NO:187, a vhCDR3 comprising SEQ ID NO:188, a vlCDR1 comprising SEQ ID NO: 190, a vlCDR2 comprising SEQ ID NO:191, and a vlCDR3 comprising SEQ ID NO:192. In some embodiments, the antibody of the present invention comprises a vhCDR1 comprising SEQ ID NO:194, a vhCDR2 comprising SEQ ID NO:195, a vhCDR3 comprising SEQ ID NO:196, a vlCDR1 comprising SEQ ID NO:198, a vlCDR2 comprising SEQ ID NO:199, and a vlCDR3 comprising SEQ ID NO:200. In some embodiments, the antibody of the present invention comprises a vhCDR1 comprising SEQ ID NO:202, a vhCDR2 comprising SEQ ID NO:203, a vlCDR1 comprising SEQ ID NO:205, a vlCDR2 comprising SEQ ID NO:206, and a vlCDR3 comprising SEQ ID NO:207. In some embodiments, the antibody of the present invention comprises a vhCDR1 comprising SEQ ID NO:209, a vhCDR2 comprising SEQ ID NO:210, a vhCDR3 comprising SEQ ID NO:211, a vlCDR1 comprising SEQ ID NO:213, a vlCDR2 comprising SEQ ID NO:214, and a vlCDR3 comprising SEQ ID NO:215.

In some embodiments, the anti-LILRB antibodies include a heavy chain variable region comprising an amino acid sequence of SEQ ID NO:1 and a light chain variable region comprising an amino acid sequence of SEQ ID NO:5. In some embodiments, the anti-LILRB antibodies include a heavy chain variable region comprising an amino acid sequence of SEQ ID NO:9 and a light chain variable region comprising an amino acid sequence of SEQ ID NO:13. In some embodiments, the anti-LILRB antibodies include a heavy chain variable region comprising an amino acid sequence of SEQ ID NO:17 and a light chain variable region comprising an amino acid sequence of SEQ ID NO:21. In some embodiments, the anti-LILRB antibodies include a heavy chain variable region comprising an amino acid sequence of SEQ ID NO:25 and a light chain variable region comprising an amino acid sequence of SEQ ID NO:29. In some embodiments, the anti-LILRB antibodies include a heavy chain variable region comprising an amino acid sequence of SEQ ID NO:33 and a light chain variable region comprising an amino acid sequence of SEQ ID NO:37. In some embodiments, the anti-LILRB antibodies include a heavy chain variable region comprising an amino acid sequence of SEQ ID NO:41 and a light chain variable region comprising an amino acid sequence of SEQ ID NO:45. In some embodiments, the anti-LILRB antibodies include a heavy chain variable region comprising an amino acid sequence of SEQ ID NO:49 and a light chain variable region comprising an amino acid sequence of SEQ ID NO:53. In some embodiments, the anti-LILRB antibodies include a heavy chain variable region comprising an amino acid sequence of SEQ ID NO:57 and a light chain variable region comprising an amino acid sequence of SEQ ID NO:61. In some embodiments, the anti-LILRB antibodies include a heavy chain variable region comprising an amino acid sequence of SEQ ID NO:65 and a light chain variable region comprising an amino acid sequence of SEQ ID NO:69. In some embodiments, the anti-LILRB antibodies include a heavy chain variable region comprising an amino acid sequence of SEQ ID NO:73 and a light chain variable region comprising an amino acid sequence of SEQ ID NO:77. In some embodiments, the anti-LILRB antibodies include a heavy chain variable region comprising an amino acid sequence of SEQ ID NO:81 and a light chain variable region comprising an amino acid sequence of SEQ ID NO:85. In some embodiments, the anti-LILRB antibodies include a heavy chain variable region comprising an amino acid sequence of SEQ ID NO:89 and a light chain variable region comprising an amino acid sequence of SEQ ID NO:93. In some embodiments, the anti-LILRB antibodies include a heavy chain variable region comprising an amino acid sequence of SEQ ID NO:97 and a light chain variable region comprising an amino acid sequence of SEQ ID NO:101. In some embodiments, the anti-LILRB antibodies include a heavy chain variable region comprising an amino acid sequence of SEQ ID NO:105 and a light chain variable region comprising an amino acid sequence of SEQ ID NO:109. In some embodiments, the anti-LILRB antibodies include a heavy chain variable region comprising an amino acid sequence of SEQ ID NO:113 and a light chain variable region comprising an amino acid sequence of SEQ ID NO:117. In some embodiments, the anti-LILRB antibodies include a heavy chain variable region comprising an amino acid sequence of SEQ ID NO:121 and a light chain variable region comprising an amino acid sequence of SEQ ID NO:125. In some embodiments, the anti-LILRB antibodies include a heavy chain variable region comprising an amino acid sequence of SEQ ID NO:129 and a light chain variable region comprising an amino acid sequence of SEQ ID NO:133. In some embodiments, the anti-LILRB antibodies include a heavy chain variable region comprising an amino acid sequence of SEQ ID NO:137 and a light chain variable region comprising an amino acid sequence of SEQ ID NO: 141. In some embodiments, the anti-LILRB antibodies include a heavy chain variable region comprising an amino acid sequence of SEQ ID NO:145 and a light chain variable region comprising an amino acid sequence of SEQ ID NO:149. In some embodiments, the anti-LILRB antibodies include a heavy chain variable region comprising an amino acid sequence of SEQ ID NO:153 and a light chain variable region comprising an amino acid sequence of SEQ ID NO:157. In some embodiments, the anti-LILRB antibodies include a heavy chain variable region comprising an amino acid sequence of SEQ ID NO:161 and a light chain variable region comprising an amino acid sequence of SEQ ID NO:165. In some embodiments, the anti-LILRB antibodies include a heavy chain variable region comprising an amino acid sequence of SEQ ID NO:169 and a light chain variable region comprising an amino acid sequence of SEQ ID NO:173. In some embodiments, the anti-LILRB antibodies include a heavy chain variable region comprising an amino acid sequence of SEQ ID NO:177 and a light chain variable region comprising an amino acid sequence of SEQ ID NO:181. In some embodiments, the anti-LILRB antibodies include a heavy chain variable region comprising an amino acid sequence of SEQ ID NO:185 and a light chain variable region comprising an amino acid sequence of SEQ ID NO:189. In some embodiments, the anti-LILRB antibodies include a heavy chain variable region comprising an amino acid sequence of SEQ ID NO:193 and a light chain variable region comprising an amino acid sequence of SEQ ID NO:197. In some embodiments, the anti-LILRB antibodies include a heavy chain variable region comprising an amino acid sequence of SEQ ID NO:201 and a light chain variable region comprising an amino acid sequence of SEQ ID NO:204. In some embodiments, the anti-LILRB antibodies include a heavy chain variable region comprising an amino acid sequence of SEQ ID NO:208 and a light chain variable region comprising an amino acid sequence of SEQ ID NO:212.

In some embodiments, the antibody comprises a constant region with an amino acid sequence at least 90% identical to a human IgG. In some embodiments, the human IgG is selected from a group consisting of IgG1, IgG2, IgG3 and IgG4. In some embodiments, the IgG is an IgG1. In some embodiments, the IgG is an IgG2.

In some embodiments, the present disclosure provides nucleic acid compositions encoding the anti-LILRB antibodies as disclosed herein.

In some embodiments, the present disclosure provides expression vector compositions comprising the nucleic acid composition as disclosed herein, wherein the first nucleic acid is contained in a first expression vector and the second nucleic acid is contained in a second expression vector.

In some embodiments, the present disclosure provides expression vector compositions comprising the nucleic acid composition as disclosed herein, wherein the first nucleic acid and the second nucleic acid are contained in a single expression vector.

In some embodiments, the present disclosure provides host cells comprising the expression vector composition as disclosed herein.

In some embodiments, the present disclosure provides methods of making an antibody comprising culturing the host cell as disclosed herein under conditions wherein the antibody is expressed, and recovering the antibody.

In some embodiments, the present disclosure provides compositions comprising the antibody as disclosed herein, and a pharmaceutical acceptable carrier or diluent.

In some embodiments, the present disclosure provides methods of modulating an immune response in a subject, the method comprising administering to the subject an effective amount of the antibody or the composition as disclosed herein.

In some embodiments, the present disclosure provides methods of treating cancer in a subject comprising administering to the subject an effective amount of the antibody or the composition as disclosed herein. In some embodiments, the cancer upregulates LILRB1 or LILRB2. In some embodiments, the antibody is combined with one or more additional therapeutic agents to treat cancer. In some embodiments, the additional therapeutic agents are other immune checkpoint inhibitors. In some embodiments, the other immune checkpoint inhibitors are selected from the group consisting of Ipilimumab, Nivolumab, Pembrolizumab, Avelumab, Durvalumab, and Atezolizumab.

In some embodiments, the present disclosure provides methods of treating an autoimmune disease in a subject comprising administering to the subject an effective amount of the antibody or the composition as disclosed herein. In some embodiments, the antibody is combined with one or more additional therapeutic agents to treat autoimmune disease.

In some embodiments, the present disclosure provides methods of treating allergic inflammation in a subject comprising administering to the subject an effective amount of the antibody or the composition as disclosed herein. In some embodiments, the antibody is combined with one or more additional therapeutics to treat allergic inflammation.

The present disclosure alos provides for the use of an antibody as described herein, including in paragraphs [0005]-[0020], according to a method as described herein, including in paragraphs [0005]-[0020].

### BRIEF DESCRIPTION OF THE DRAWINGS

The invention may be best understood from the following detailed description when read in conjunction with the accompanying drawings. Included in the drawings are the following figures:

FIG. 1A-1B show the expression of primary monocyte populations in the form of two-dimensional flow cytometry (FCM) representations called quantile contour plots (probability plots). Peripheral blood from healthy human donors was obtained from the Hematology Malignancy Tissue Bank at the University Health Network. Peripheral blood mononuclear cells (PBMCs) were prepared from the blood by density gradient centrifugation using Ficoll-Paque PLUS by manufacturer's instructions (GE Healthcare Life Sciences). Monocytes were purified from PBMCs using Pan Monocyte Isolation Kit, Human by manufacturer's instructions (Miltenyi Biotech). PBMCs and monocytes were used fresh or frozen at 20×10⁶ cells/vial and 5×10⁶ cells/vial, respectively, in 90% heat-inactivated fetal bovine serum (FBS) and 10% dimethylsulfoxide (DMSO) and stored in liquid nitrogen until use. Purified monocytes were stained with Ghost Dye Violet 510 viability dye (Tonbo Biosciences) and CD3, CD19, CD56, CD11b, CD14 and CD16 antibodies, and analyzed on a BD LRSFortessa flow cytometer (BD Biosciences). The plot depicts expression of classical monocytes (C) by gated CD3⁻CD19⁻CD56⁻CD11b⁺CD14⁺⁺CD16⁻ cells, intermediate monocytes (INT) by gated CD3⁻CD19⁻CD56⁻CD11b⁺CD14⁺CD16⁺ cells and non-classical monocytes (NC) by gated CD3⁻CD19⁻CD56⁻CD11b⁺CD14⁺CD16⁺⁺ cells. Percentages of gated cells within the purified sample are depicted inside the plot. In FIG. 1B, purified monocytes were stained as in FIG. 1A and with Pacific Blue anti-LILRB1 (clone GHI/75; BioLegend, Inc.) or APC anti-LILRB2 (clone 287219; BD Biosciences) antibodies, and analyzed on a BD LRSFortessa flow cytometer (BD Biosciences). Plots depict relative LILRB1 (left panel) or LILRB2 (right panel) expression by gated classical (C), intermediate (INT) and non-classical (NC) monocytes. Specificity of the LILRB1 or LILRB2 staining was determined by staining all of the above cell subsets with an IgG1 isotype control antibody (BioLegend, Inc.). Data are representative of several independent experiments utilizing different human monocyte samples.

FIG. 2A-2C show the HLA-G binding profile of exemplary anti-LILRB antibodies with respect to primary human monocytes as determined by flow cytometry. Purified monocytes were washed with FACS buffer (DPBS+2% BSA+2 mM EDTA), stained with Ghost Dye Violet 510 viability dye (Tonbo Biosciences) and Fc receptor (FcR) blocked with Human TruStain FcX (BioLegend, Inc.) and normal mouse serum. 2 μg each anti-LILRB antibody or isotype control antibody (BioLegend, Inc.) was added to 100,000 monocytes in 100 μL FACS buffer for 30 minutes at 4° C. 1 μL of PE HLA-G*01:01 tetramer (Creative Biolabs) was then added to each sample and stained, protected from light, for 30 minutes at 4° C. Monocytes were then stained with CD3, CD19, CD56, CD11b, CD14 and CD16 antibodies for 30 minutes at 4° C. for cell surface phenotyping. Samples were washed with FACS buffer, centrifuged, resuspended in FACS buffer and analyzed on a BD LRSFortessa flow cytometer (BD Biosciences). Data are representative of several independent experiments utilizing different monocyte samples (circle and square), and are reported as the average percent mean fluorescence intensity (MFI) of PE HLA-G*01:01 tetramer binding in the presence of the anti-LILRB antibodies relative to tetramer alone±standard error of duplicate samples. PE HLA-G*01:01 tetramer binding to classical, intermediate and non-classical monocytes is shown in FIG. 2A, FIG. 2B and FIG. 2C, respectively. Five out of the fourteen anti-LILRB1 antibodies and six out of the twelve anti-LILRB2 antibodies blocked HLA-G tetramer binding to monocytes. Nine out of the fourteen anti-LILRB1 antibodies and five out of the twelve anti-LILRB2 antibodies increased HLA-G tetramer binding to monocytes. None of the isotype control antibodies affected HLA-G tetramer from binding monocytes.

FIG. 3A-3C show the HLA-G binding profile of exemplary HLA-G blocking anti-LILRB antibodies with respect to primary human monocytes as determined by flow cytometry. Data are representative of several independent experiments utilizing different monocyte samples (circle and square) and are reported as the average percent mean fluorescence intensity (MFI) of PE HLA-G*01:01 tetramer binding in the presence of the anti-LILRB antibodies relative to tetramer alone±standard error of duplicate samples. PE HLA-G*01:01 tetramer binding to classical, intermediate and non-classical monocytes is shown in FIG. 3A, FIG. 3B and FIG. 3C, respectively. None of the isotype control antibodies affected HLA-G tetramer from binding monocytes.

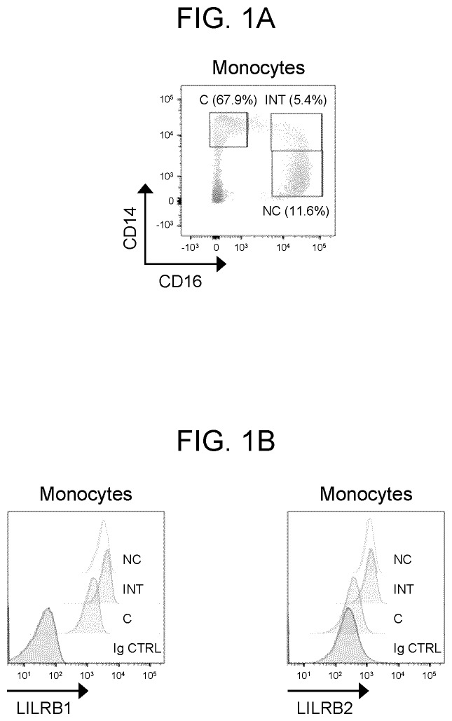

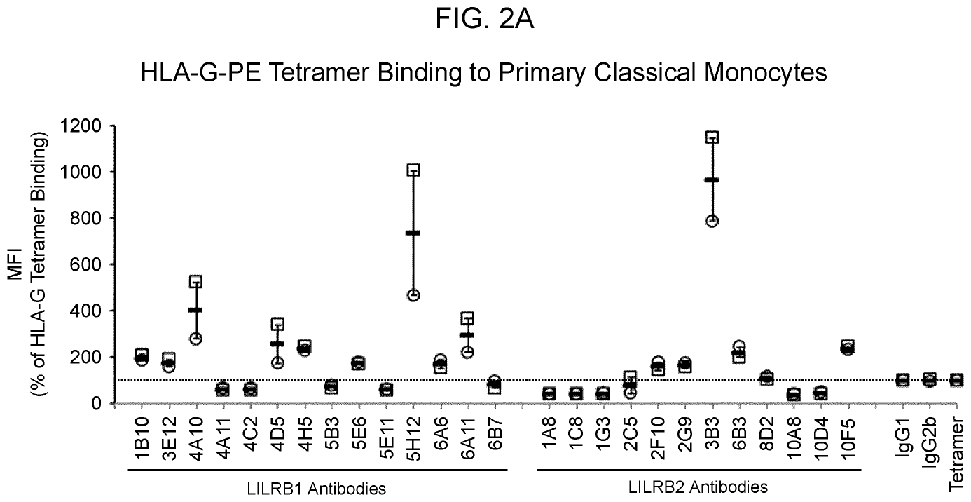

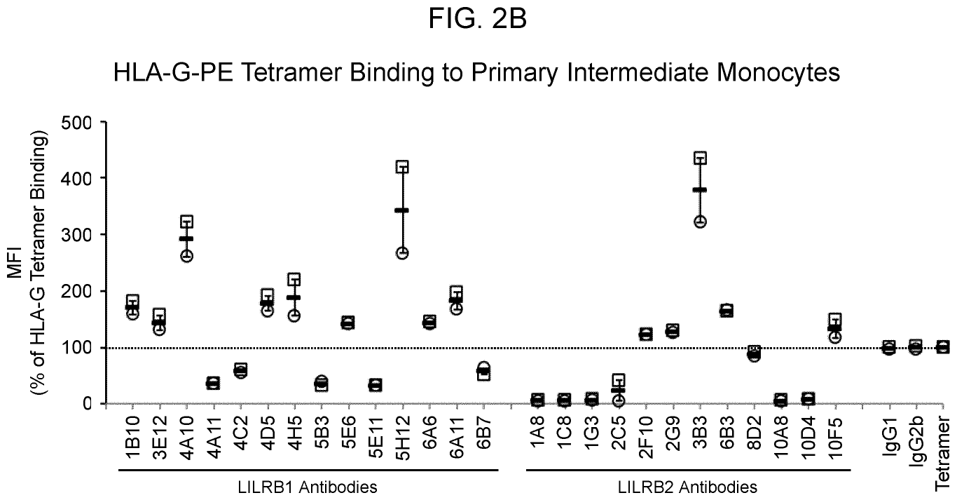

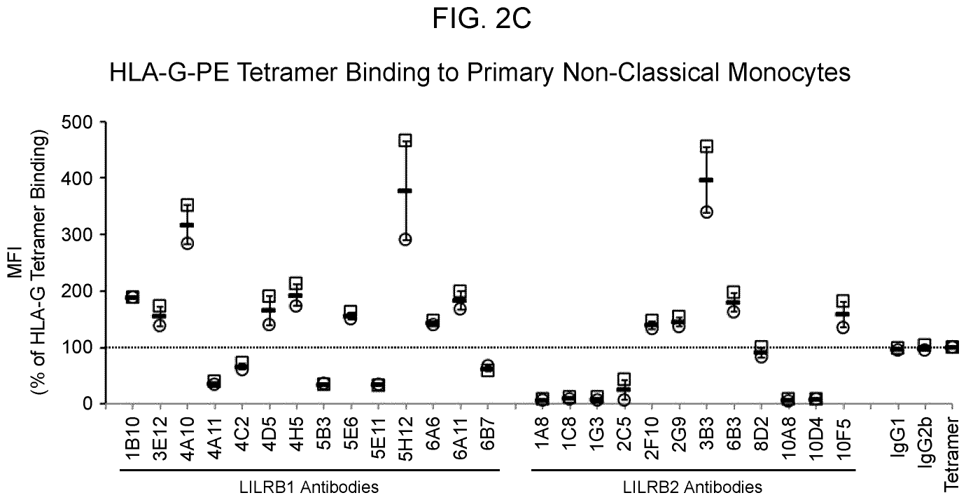

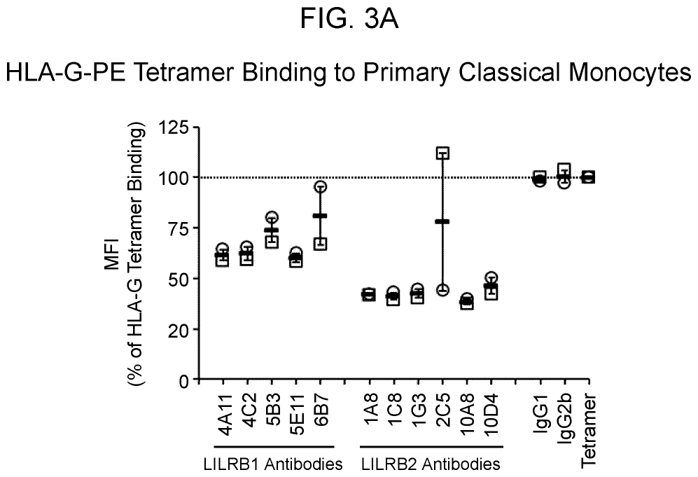

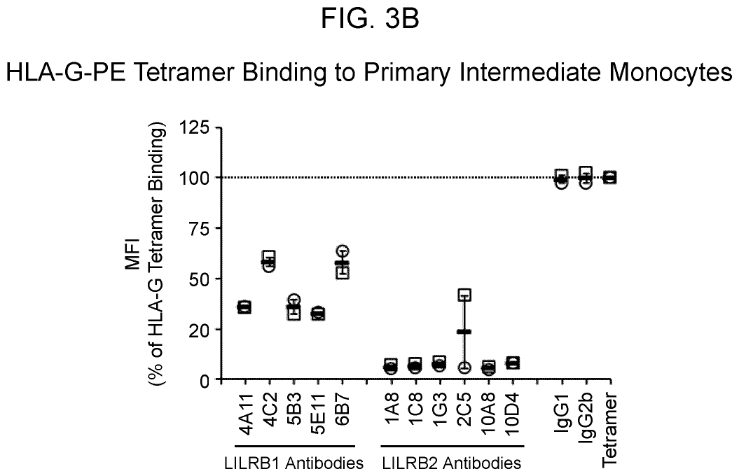

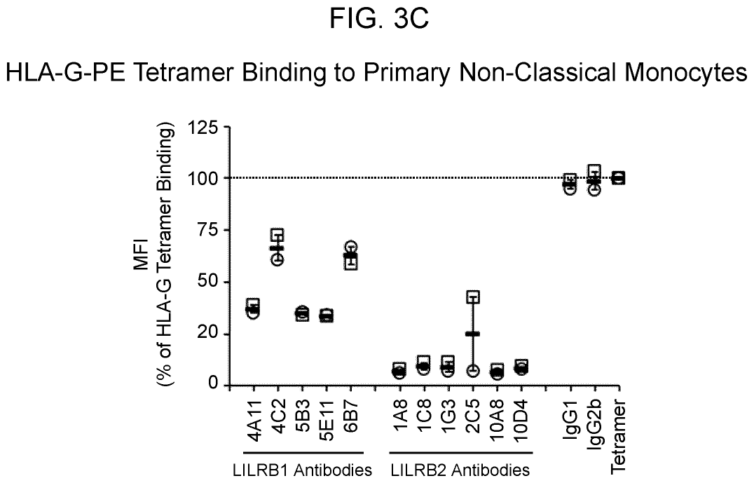

### DETAILED DESCRIPTION

The present disclosure provides novel anti-LILRB1 and anti-LILRB2 antibodies. In some embodiments, the antibodies described herein act to modulate an immune response in a subject, and, for example, to treat cancer or an autoimmune disease. In some embodiments the antibodies described herein act to treat allergic inflammation.

To facilitate an understanding of the present invention, a number of terms and phrases are defined below.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of 20% or ±10%, more preferably ±5%, even more preferably ±1%, and still more preferably ±0.1% from the specified value, as such variations are appropriate to perform the disclosed methods.

By “antigen binding domain” or “ABD” herein is meant a set of six Complementary Determining Regions (CDRs) that, when present as part of a polypeptide sequence, specifically binds a target antigen as discussed herein. Thus, an “antigen binding domain” binds a target antigen as outlined herein. As is known in the art, these CDRs are generally present as a first set of variable heavy CDRs (vhCDRs or VHCDRs or CDR-HC) and a second set of variable light CDRs (vlCDRs or VLCDRs or CDR-LC), each comprising three CDRs: vhCDRT, vhCDR2, vhCDR3 for the heavy chain and vlCDR1, vlCDR2 and vlCDR3 for the light chain. The CDRs are present in the variable heavy and variable light domains, respectively, and together form an Fv region. Thus, in some cases, the six CDRs of the antigen binding domain are contributed by a variable heavy and variable light chain. In a “Fab” format, the set of 6 CDRs are contributed by two different polypeptide sequences, the variable heavy domain (vh or VH; containing the vhCDRT, vhCDR2 and vhCDR3) and the variable light domain (vl or VL; containing the vlCDR1, vlCDR2 and vlCDR3), with the C-terminus of the vh domain being attached to the N-terminus of the CH1 domain of the heavy chain and the C-terminus of the vl domain being attached to the N-terminus of the constant light domain (and thus forming the light chain). In a scFv format, the VH and VL domains are covalently attached, generally through the use of a linker as outlined herein, into a single polypeptide sequence, which can be either (starting from the N-terminus) vh-linker-vl or vl-linker-vh, with the former being generally preferred (including optional domain linkers on each side, depending on the format used. As is understood in the art, the CDRs are separated by framework regions in each of the variable heavy and variable light domains: for the light variable region, these are FR1-vlCDR1-FR2-vlCDR2-FR3-vlCDR3-FR4, and for the heavy variable region, these are FR1-vhCDR1-FR2-vhCDR2-FR3-vhCDR3-FR4, with the framework regions showing high identity to human germline sequences. Antigen binding domains of the invention include, Fab, Fv and scFv.

The term “antibody” is used in the broadest sense and includes, for example, an intact immunoglobulin or an antigen binding portion. Antigen binding portions may be produced by recombinant DNA techniques or by enzymatic or chemical cleavage of intact antibodies. Thus the term antibody includes traditional tetrameric antibodies of two heavy chains and two light chains, as well as antigen binding fragments such as Fv, Fab and scFvs. In some cases, the invention provides bispecific antibodies that include at least one antigen binding domain as outlined herein.

By “modification” herein is meant an amino acid substitution, insertion, and/or deletion in a polypeptide sequence or an alteration to a moiety chemically linked to a protein. For example, a modification may be an altered carbohydrate or PEG structure attached to a protein. By “amino acid modification” herein is meant an amino acid substitution, insertion, and/or deletion in a polypeptide sequence. For clarity, unless otherwise noted, the amino acid modification is always to an amino acid coded for by DNA, e.g., the 20 amino acids that have codons in DNA and RNA.

By “amino acid substitution” or “substitution” herein is meant the replacement of an amino acid at a particular position in a parent polypeptide sequence with a different amino acid. In particular, in some embodiments, the substitution is to an amino acid that is not naturally occurring at the particular position, either not naturally occurring within the organism or in any organism. For example, the substitution M252Y refers to a variant polypeptide, in this case an Fc variant, in which the methionine at position 252 is replaced with tyrosine. For clarity, a protein which has been engineered to change the nucleic acid coding sequence but not change the starting amino acid (for example exchanging CGG (encoding arginine) to CGA (still encoding arginine) to increase host organism expression levels) is not an “amino acid substitution”; that is, despite the creation of a new gene encoding the same protein, if the protein has the same amino acid at the particular position that it started with, it is not an amino acid substitution.

By “variant protein” or “protein variant”, or “variant” as used herein is meant a protein that differs from that of a parent protein by virtue of at least one amino acid modification. Protein variant may refer to the protein itself, a composition comprising the protein, or the amino sequence that encodes it. Preferably, the protein variant has at least one amino acid modification compared to the parent protein, e.g., from about one to about seventy amino acid modifications, and preferably from about one to about five amino acid modifications compared to the parent. As described below, in some embodiments the parent polypeptide, for example an Fc parent polypeptide, is a human wild-type sequence, such as the Fc region from IgG1, IgG2, IgG3 or IgG4. The protein variant sequence herein will preferably possess at least about 80% identity with a parent protein sequence, and most preferably at least about 90% identity, more preferably at least about 95%-98%-99% identity. Variant protein can refer to the variant protein itself, compositions comprising the protein variant, or the DNA sequence that encodes it.

Accordingly, by “antibody variant” or “variant antibody” as used herein is meant an antibody that differs from a parent antibody by virtue of at least one amino acid modification, “IgG variant” or “variant IgG” as used herein is meant an antibody that differs from a parent IgG (again, in many cases, from a human IgG sequence) by virtue of at least one amino acid modification, and “immunoglobulin variant” or “variant immunoglobulin” as used herein is meant an immunoglobulin sequence that differs from that of a parent immunoglobulin sequence by virtue of at least one amino acid modification. “Fc variant” or “variant Fc” as used herein is meant a protein comprising an amino acid modification in an Fc domain. The Fc variants of the present invention are defined according to the amino acid modifications that compose them. Thus, for example M252Y or 252Y is an Fc variant with the substitution tyrosine at position 252 relative to the parent Fc polypeptide, wherein the numbering is according to the EU index. Likewise, M252Y/S254T/T256E defines an Fc variant with the substitutions M252Y, S254T and T256E relative to the parent Fc polypeptide. The identity of the wild type amino acid may be unspecified, in which case the aforementioned variant is referred to as 252Y/254T/256E. It is noted that the order in which substitutions are provided is arbitrary, that is to say that, for example, 252Y/254T/256E is the same Fc variant as 254T/252Y/256E, and so on. For all positions discussed in the present invention that relate to antibodies, unless otherwise noted, amino acid position numbering is according to Kabat for the variable region numbering and is according to the EU index for the constant regions, including the Fc region. The EU index or EU index as in Kabat or EU numbering scheme refers to the numbering of the EU antibody (Edelman et al., 1969, Proc Natl Acad Sci USA 63:78-85, hereby entirely incorporated by reference.) The modification can be an addition, deletion, or substitution. Substitutions can include naturally occurring amino acids and, in some cases, synthetic amino acids.

As used herein, “protein” herein is meant at least two covalently attached amino acids, which includes proteins, polypeptides, oligopeptides and peptides. The peptidyl group may comprise naturally occurring amino acids and peptide bonds.

By “Fab” or “Fab region” as used herein is meant the polypeptide that comprises the VH, CH1, VL, and CL immunoglobulin domains. Fab may refer to this region in isolation, or this region in the context of a full-length antibody, antibody fragment or Fab fusion protein.

By “Fv” or “Fv fragment” or “Fv region” as used herein is meant a polypeptide that comprises the VL and VH domains of a single antigen binding domain (ABD). As will be appreciated by those in the art, these generally are made up of two chains, or can be combined (generally with a linker as discussed herein) to form a scFv.

By “amino acid” and “amino acid identity” as used herein is meant one of the 20 naturally occurring amino acids that are coded for by DNA and RNA.

By “parent polypeptide” as used herein is meant a starting polypeptide that is subsequently modified to generate a variant. The parent polypeptide may be a naturally occurring polypeptide, or a variant or engineered version of a naturally occurring polypeptide. Parent polypeptide may refer to the polypeptide itself, compositions that comprise the parent polypeptide, or the amino acid sequence that encodes it. Accordingly, by “parent immunoglobulin” as used herein is meant an unmodified immunoglobulin polypeptide that is modified to generate a variant, and by “parent antibody” as used herein is meant an unmodified antibody that is modified to generate a variant antibody. It should be noted that “parent antibody” includes known commercial, recombinantly produced antibodies as outlined below.

By “heavy constant region” herein is meant the CH1-hinge-CH2-CH3 portion of an antibody, generally from human IgG1, IgG2 or IgG4.

By “target antigen” as used herein is meant the molecule that is bound specifically by the variable region of a given antibody. In the present case, the target antigen is a LILRB protein.

By “target cell” as used herein is meant a cell that expresses a target antigen.

By “variable region” as used herein is meant the region of an immunoglobulin that comprises one or more Ig domains substantially encoded by any of the V.kappa., V.lamda., and/or VH genes that make up the kappa, lambda, and heavy chain immunoglobulin genetic loci, respectively.

By “wild type or WT” herein is meant an amino acid sequence or a nucleotide sequence that is found in nature, including allelic variations. A WT protein has an amino acid sequence or a nucleotide sequence that has not been intentionally modified.

By “position” as used herein is meant a location in the sequence of a protein. Positions may be numbered sequentially, or according to an established format, for example the EU index for antibody numbering.

By “residue” as used herein is meant a position in a protein and its associated amino acid identity. For example, Asparagine 297 (also referred to as Asn297 or N297) is a residue at position 297 in the protein sequence.

The antibodies of the present invention are generally recombinant. “Recombinant” means the antibodies are generated using recombinant nucleic acid techniques in exogenous host cells.

“Percent (%) amino acid sequence identity” with respect to a protein sequence is defined as the percentage of amino acid residues in a candidate sequence that are identical with the amino acid residues in the specific (parental) sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. One particular program is the ALIGN-2 program outlined at paragraphs [0279] to [0280] of US Pub. No. 20160244525, hereby incorporated by reference. Another approximate alignment for nucleic acid sequences is provided by the local homology algorithm of Smith and Waterman, Advances in Applied Mathematics, 2:482-489 (1981). This algorithm can be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986).

An example of an implementation of this algorithm to determine percent identity of a sequence is provided by the Genetics Computer Group (Madison, WI) in the “BestFit” utility application. The default parameters for this method are described in the Wisconsin Sequence Analysis Package Program Manual, Version 8 (1995) (available from Genetics Computer Group, Madison, WI). Another method of establishing percent identity in the context of the present invention is to use the MPSRCH package of programs copyrighted by the University of Edinburgh, developed by John F. Collins and Shane S. Sturrok, and distributed by IntelliGenetics, Inc. (Mountain View, CA). From this suite of packages, the Smith-Waterman algorithm can be employed where default parameters are used for the scoring table (for example, gap open penalty of 12, gap extension penalty of one, and a gap of six). From the data generated the “Match” value reflects “sequence identity.” Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs can be found at the internet address located by placing http:// in front of blast.ncbi.nlm.nih.gov/Blast.cgi.

The degree of identity between an amino acid sequence of the present invention (“invention sequence”) and the parental amino acid sequence is calculated as the number of exact matches in an alignment of the two sequences, divided by the length of the “invention sequence,” or the length of the parental sequence, whichever is the shortest. The result is expressed in percent identity.

In some embodiments, two or more amino acid sequences are at least 50%, 60%, 70%, 80%, or 90% identical. In some embodiments, two or more amino acid sequences are at least 95%, 97%, 98%, 99%, or even 100% identical.

“Specific binding” or “specifically binds to” or is “specific for” a particular antigen or an epitope means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target.

The term “Kassoc” or “Ka”, as used herein, is intended to refer to the association rate of a particular antibody-antigen interaction, whereas the term “Kdis” or “Kd,” as used herein, is intended to refer to the dissociation rate of a particular antibody-antigen interaction. The term “K_{D}”, as used herein, is intended to refer to the dissociation constant, which is obtained from the ratio of Kd to Ka (i.e., Kd/Ka) and is expressed as a molar concentration (M). K_{D} values for antibodies can be determined using methods well established in the art. In some embodiments, the method for determining the K_{D} of an antibody is by using surface plasmon resonance, for example, by using a biosensor system such as a BIACORE® system. In some embodiments, the K_{D} of an antibody is determined by Bio-Layer Interferometry. In some embodiments, the K_{D} value is measured with the immobilized. In other embodiments, the K_{D} value is measured with the antibody (e.g., parent mouse antibody, chimeric antibody, or humanized antibody variants) immobilized. In certain embodiments, the K_{D} value is measured in a bivalent binding mode. In other embodiments, the K_{D} value is measured in a monovalent binding mode.

A “disease” includes a state of health of an animal, including a human, wherein the animal cannot maintain homeostasis, and wherein if the disease is not ameliorated then the animal's health continues to deteriorate.

In contrast, a “disorder” in an animal, including a human, includes a state of health in which the animal is able to maintain homeostasis, but in which the animal's state of health is less favorable than it would be in the absence of the disorder. Left untreated, a disorder does not necessarily cause a further decrease in the animal's state of health.

The terms “treatment”, “treating”, “treat”, and the like, refer to obtaining a desired pharmacologic and/or physiologic effect. The effect may be prophylactic in terms of completely or partially preventing a disease or symptom thereof or reducing the likelihood of a disease or symptom thereof and/or may be therapeutic in terms of a partial or complete cure for a disease and/or adverse effect attributable to the disease. “Treatment”, as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development or progression; and (c) relieving the disease, i.e., causing regression of the disease and/or relieving one or more disease symptoms. “Treatment” is also meant to encompass delivery of an agent in order to provide for a pharmacologic effect, even in the absence of a disease or condition. For example, “treatment” encompasses delivery of a composition that can elicit an immune response or confer immunity in the absence of a disease condition, e.g., in the case of a vaccine.

As used herein, the term “mammal” refers to any mammal, including, but not limited to, mammals of the order Rodentia, such as mice and hamsters, and mammals of the order Logomorpha, such as rabbits. In some embodiments, the mammals are from the order Carnivora, including felines (cats) and canines (dogs). In some embodiments, the mammals are from the order Artiodactyla, including bovines (cows) and swines (pigs) or of the order Perssodactyla, including Equines (horses). It is most preferred that the mammals are of the order Primates, Ceboids, or Simoids (monkeys) or of the order Anthropoids (humans and apes). In some embodiments, the mammal is a human. In some embodiments, the mammal is cynomolgus monkey.

The term “regression,” as well as words stemming therefrom, as used herein, does not necessarily imply 100% or complete regression. Rather, there are varying degrees of regression of which one of ordinary skill in the art recognizes as having a potential benefit or therapeutic effect. In this respect, the disclosed methods can provide any amount of any level of regression of a cancer in a mammal. Furthermore, the regression provided by the inventive method can include regression of one or more conditions or symptoms of the disease, e.g., a cancer. Also, for purposes herein, “regression” can encompass delaying the onset of the disease, delaying the onset of a symptom, and/or delaying the onset of a condition thereof. With respect to progressive diseases and disorders, “regression” can encompass slowing the progression of the disease or disorder, slowing the progression of a symptom of the disease or disorder, and/or slowing the progression of a condition thereof.

An “effective amount” or “therapeutically effective amount” of a composition includes that amount of the composition which is sufficient to provide a beneficial effect to the subject to which the composition is administered. An “effective amount” of a delivery vehicle includes that amount sufficient to effectively bind or deliver a composition.

By “individual” or “host” or “subject” or “patient” is meant any mammalian subject for whom diagnosis, treatment, or therapy is desired, particularly humans. Other subjects may include cynomolgus monkey, cattle, dogs, cats, guinea pigs, rabbits, rats, mice, horses, and so on.

The term “in combination with” as used herein refers to uses where, for example, a first therapy is administered during the entire course of administration of a second therapy; where the first therapy is administered for a period of time that is overlapping with the administration of the second therapy, e.g., where administration of the first therapy begins before the administration of the second therapy and the administration of the first therapy ends before the administration of the second therapy ends; where the administration of the second therapy begins before the administration of the first therapy and the administration of the second therapy ends before the administration of the first therapy ends; where the administration of the first therapy begins before administration of the second therapy begins and the administration of the second therapy ends before the administration of the first therapy ends; where the administration of the second therapy begins before administration of the first therapy begins and the administration of the first therapy ends before the administration of the second therapy ends. As such, “in combination” can also refer to regimen involving administration of two or more therapies. “In combination with” as used herein also refers to administration of two or more therapies which may be administered in the same or different formulations, by the same or different routes, and in the same or different dosage form type.

The term “allergic inflammation” as used herein refers to a local or general hypersensitivity reaction to at least one particular allergen. “Allergic inflammation” symptoms can vary greatly in effects and intensity.

“Encoding” includes the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if, for example, transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

The term “nucleic acid” includes RNA or DNA molecules having more than one nucleotide in any form including single-stranded, double-stranded, oligonucleotide or polynucleotide. The term “nucleotide sequence” includes the ordering of nucleotides in an oligonucleotide or polynucleotide in a single-stranded form of nucleic acid.

By “nucleic acid construct” it is meant a nucleic acid sequence that has been constructed to comprise one or more functional units not found together in nature. Examples include circular, linear, double-stranded, extrachromosomal DNA molecules (plasmids), cosmids (plasmids containing COS sequences from lambda phage), viral genomes including non-native nucleic acid sequences, and the like.

The term “operably linked” as used herein includes a polynucleotide in functional relationship with a second polynucleotide, e.g., a single-stranded or double-stranded nucleic acid moiety comprising the two polynucleotides arranged within the nucleic acid moiety in such a manner that at least one of the two polynucleotides is able to exert a physiological effect by which it is characterized, upon the other. By way of example, a promoter operably linked to the coding region of a gene is able to promote transcription of the coding region. The order specified when indicating operably linkage is not important. For example, the phrases: “the promoter is operably linked to the nucleotide sequence” and “the nucleotide sequence is operably linked to the promoter” are used interchangeably herein and are considered equivalent. In some cases, when the nucleic acid encoding the desired protein further comprises a promoter/regulatory sequence, the promoter/regulatory sequence is positioned at the 5′ end of the desired protein coding sequence such that it drives expression of the desired protein in a cell.

The terms “oligonucleotide,” “polynucleotide,” and “nucleic acid molecule”, used interchangeably herein, refer to a polymeric forms of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The backbone of the polynucleotide can comprise sugars and phosphate groups (as may typically be found in RNA or DNA), or modified or substituted sugar or phosphate groups.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see e.g., Martin, Remington's Pharmaceutical Sciences, 15th Ed., Mack Publ. Co., Easton, PA [1975].

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

Various aspects of the invention are set forth below in sections; however, aspects of the invention described in one particular section are not to be limited to any particular section.

I. Antibodies

The present disclosure provides novel anti-LILRB1 and anti-LILRB2 antibodies. Such antibodies bind to and/or affect the functional properties of human LILRB1 or human LILRB2 respectively. Table 1 lists peptide sequences of heavy chain variable regions and light chain variable regions that, in combination as designated in Table 1, are LILRB1 antibodies. Table 2 lists peptide sequences of heavy chain variable regions and light chain variable regions that, in combination as designated in Table 1, are LILRB2 antibodies. For both the LILRB1 and LILRB2 antibodies disclosed herein, in some embodiments, the heavy chain variable region and the light chain variable region are arranged in a Fab format. In some embodiments, the heavy chain variable region and the light chain variable region are fused together to from an scFv. CDR sequences were determined by IgBLAST (Ye, J., Ma, N., Madden, T. L., and Ostell, J. M. (2013) IgBLAST: an immunoglobulin variable domain sequence analysis tool. Nucleic Acids Res. 41 (Web Server Issue), W34-W40. doi: 10.1093/nar/gkt382)

TABLE 1
(LILRB1 antibodies)

| | Heavy chain variable region | Light chain variable region |
|---|---|---|
| Clone | amino acid sequence | amino acid sequence |
| 1B10 | MGWSYIILFLVATATGVHSQVQL | MDFQVQIFSFLLMSASVIMSRG |
| | QQPGAELVKPGASVKLSCKASG | QIVLTQSPALMSASPGEKVTMT |
| | YTFTNYWVQWVKQRPGQGLEW | CSASSSVSHMYWYQQKPRSSP |
| | IGMIHPNSGSTNSNEKFKNKATL | KPWIYLTSNLASGVPARFSGSG |
| | TVDRSSSTAYMQLSRLTSEDSAV | SGTSYSLTISNMEAEDAATYYC |
| | YYCARGDDGYLYYFDYWGQGT | QQWSSYAFTFGSGTKLEIKRAD |
| | TLTVSSAKTTPPSVYPLAPGCGD | AAPTVSIFPPSSEQLTSGGASVV |
| | TTGSSVTLGCLVKGYFPESVTVT | CFLNNFYPRDINVKWKIDGSER |
| | WNSGSLSSSVHTFPALLQSGLYT | QNGVLNSWTDQDSKDSTYSMS |
| | MSSSVTVPSSTWPSQTVTCSVAH | STLTLTKDEYERHNSYTCEATH |
| | PASSTTVDKKLEPSGPISTINPCPP | KTSTSP |
| | CKECHKCPAPNLEGGPSVFIFPPN | SEQ ID NO: 5 (kappa) |
| | IKDVLMISLTPKVTCVVVDVSED | CDR1 (SEQ ID NO: 6)-SSVSH |
| | DQ | CDR2 (SEQ ID NO: 7)-LTS |
| | SEQ ID NO: 1 (IgG2b) | CDR3 (SEQ ID NO: 8)-QQWSS |
| | CDR1 (SEQ ID NO: 2)- | |
| | GYTFTNYW | |
| | CDR2 (SEQ ID NO: 3)- | |
| | IHPNSGST | |
| | CDR3 (SEQ ID NO: 4)- | |
| | AR | |
| 3E12 | MEWTWVFLFLLSVTAGVHSQVQ | MHFQVQIFSFLLISASVIMSRGQ |
| | LQQSGAELMKPGASVKLSCKAT | FILTQSPAIMSASPGEKVTITCS |
| | GYTFTGYWIEWVKQRPGHGLEW | ASSSVSYMHWFQQKPGTSPKL |
| | IGEILLGSGNTNYNENFKGKATL | WIYSTSNLASGVPARFSGSGSG |
| | TADTSSNTAYMQLSSLTTEDSAI | TSYSLTISRMEAEDAATYYCQR |
| | YYCASTHDGYWGQGTTLTVSSA | RSSYPFTFGSGTKLEIKRADAA |
| | KTTAPSVYPLAPVCGGTTGSSVT | PTVSIFPPSSEQLTSGGASVVCF |
| | LGCLVKGYFPEPVTLTWNSGSLS | LNNFYPRDINVKWKIDGSERQ |
| | SGVHTFPALLQSGLYTLSSSVTV | NGVLNSWTDQDSKDSTYSMSS |
| | TSNTWPSQTITCNVAHPASSTKV | TLTLTKDEYERHNSYTCEATH |
| | DKKIEPRVPITQNPCPPLKECPPC | KTSTSP |
| | AAPDLLGGPSVFIFPPKIKDVLMI | SEQ ID NO: 13 (kappa) |
| | SLSPMVTCVVVDVSEDDQ | CDR1 (SEQ ID NO: 14)- |
| | SEQ ID NO: 9 (IgG2c) | SSVSY |
| | CDR1 (SEQ ID NO: 10)- | CDR2 (SEQ ID NO: 15)- |
| | GYTFTGYW | STS |
| | CDR2 (SEQ ID NO: 11)- | CDR3 (SEQ ID NO: 16)- |
| | ILLGSGNT | QRRSSYP |
| | CDR3 (SEQ ID NO: 12)- | |
| | A | |
| 4C2 | MGWSYIILFLVATATGVHSQVQL | MRVLAELLGLLLFCFLGVRCDI |
| | QQPGAALVKPGASVKLSCKASG | QMNQSPSSLSASLGDTITITCHA |
| | YTFTSYWMHWVKQRPGQGLEW | RQNINVWLSWYQQKPGNIPKL |
| | IGMIHPNSGSTNYNEKFRSEATLT | LIYKASNLHTGVSSRFSGSGSG |
| | VDKSSSTVYMQLSSLTSEDSAVY | TGFTLTISSLQPEDIATYYCQQG |
| | YCARSGDAYYDGLDYWGQGTS | QSYPWTFGGGTKLEIKRADAA |
| | VTVSSAKTTPPSVYPLAPGSAAQ | PTVSIFPPSSEQLTSGGASVVCF |
| | TNSMVTLGCLVKGYFPEPVTVT | LNNFYPRDINVKWKIDGSERQ |
| | WNSGSLSSGVHTFPAVLQSDLYT | NGVLNSWTDQDSKDSTYSMSS |
| | LSSSVTVPSSTWPSQTVTCNVAH | TLTLTKDEYERHNSYTCEATH |
| | PASSTKVDKKIVPRDCGCKPCIC | KTSTSP |
| | TVPEVSSVFIFPPKPKDVLTITLTP | SEQ ID NO: 21 (kappa) |
| | KVTCVVVDISKDDQ | CDR1 (SEQ ID NO: 22)- |
| | SEQ ID NO: 17 (IgG1) | QNINVW |
| | CDR1 (SEQ ID NO: 18)- | CDR2 (SEQ ID NO: 23)- |
| | GYTFTSYW | KAS |
| | CDR2 (SEQ ID NO: 19)- | CDR3 (SEQ ID NO: 24)- |
| | IHPNSGST | QQGQSYP |
| | CDR3 (SEQ ID NO: 20)- | |
| | AR | |
| 4A10 | MGWSYIILSLVATATGVHSQVQL | MRCLAEFLGLLVLWIPGAIGDI |
| | QQPGAELVKPGASVKLSCKASG | VMTQAAPSVPVTPGESVSISCR |
| | YTFTNYWMYWVKQRPGQGLEW | SSKSLLHSYGNTHLYWFLQRP |
| | IGMIHPNSDSTNYNEKFKNKATL | GQSPQLLIYRMSNLASGVPDRF |
| | TVDRSSSTAYMQLSSLTSEDSAV | SGSGSGTAFTLRISRVEAEDVG |
| | YCCARGEDNYDYVMDYWGQGT | VYYCMQHLEYPLTFGAGTKLE |
| | SVTVSSAKTTPPSVYPLAPGSAA | LKRADAAPTVSIFPPSSEQLTSG |
| | QTNSMVTLGCLVKGYFPEPVTV | GASVVCFLNNFYPRDINVKWKI |
| | TWNSGSLSSGVHTFPAVLQSDLY | DGSERQNGVLNSWTDQDSKDS |
| | TLSSSVTVPSSTWPSQTVTCNVA | TYSMSSTLTLTKDEYERHNSYT |
| | HPASSTKVDKKIVPRDCGCKPCI | CEATHKTSTSP |
| | CTVPEVSSVFIFPPKPKDVLTITLT | SEQ ID NO: 29 (kappa) |
| | PKVTCVVVDISKDDQ | CDR1 (SEQ ID NO: 30)- |
| | SEQ ID NO: 25 (IgG1) | SKSLLHSYGNTH |
| | CDR1 (SEQ ID NO: 26)- | CDR2 (SEQ ID NO: 31)- |
| | GYTFTNYW | RMS |
| | CDR2 (SEQ ID NO: 27)- | CDR3 (SEQ ID NO: 32)- |
| | MIHPNSDS | MQHLEYP |
| | CDR3 (SEQ ID NO: 28)- | |
| | AR | |
| 4A11 | MGWSWIFFFLLSGTAGVHCQVQ | MGIKMETHSQVFVYMLLWLSG |
| | LKQSGAELVRPGTSVKLSCKASG | VEGDIVMTQSHKFMSTSVGDR |
| | YTFTDYYINWVKQRPGQGLEWI | VSITCKASQDVNTAVAWYQQK |
| | ARIYPGSGNTFYNEKFQDKATLT | PGQSPKLLIYWASTRHTGVPDR |
| | AEKSSSTAYMQLSSLTSEDSAVY | FTGSGSGTDFTLTISNVQSEDLA |
| | FCANTPSYGSSHWYFDVWGTGT | DYFCQQYSTYPRTFGGGTKLEI |
| | TVTVSSAKTTPPSVYPLAPGSAA | KRADAAPTVSIFPPSSEQLTSGG |
| | QTNSMVTLGCLVKGYFPEPVTV | ASVVCFLNNFYPRDINVKWKID |
| | TWNSGSLSSGVHTFPAVLQSDLY | GSERQNGVLNSWTDQDSKDST |
| | TLSSSVTVPSSTWPSQTVTCNVA | YSMSSTLTLTKDEYERHNSYTC |
| | HPASSTKVDKKIVPRDCGCKPCI | EATHKTSTSP |
| | CTVPEVSSVFIFPPKPKDVLTITLT | SEQ ID NO: 37 (kappa) |
| | PKVTCVVVDISKDDQ | CDR1 (SEQ ID NO: 38)- |
| | SEQ ID NO: 33 (IgG1) | QDVNTA |
| | CDR1 (SEQ ID NO: 34)- | CDR2 (SEQ ID NO: 39)- |
| | GYTFTDYY | WAS |
| | CDR2 (SEQ ID NO: 35)- | CDR3 (SEQ ID NO: 40)- |
| | IYPGSGNT | QQYSTYP |
| | CDR3 (SEQ ID NO: 36)- | |
| | A | |
| 4D5 | MGWSCIILFLVATATGVHSQVQL | MSVPTQVLGLLLLWLTDARCD |
| | QQPGAELVRPGSSVKLSCKASGY | IQMTQSPASLSVSVGETVTITCR |
| | TFTSYWMDWVKQRPGQGLEWI | ASENIYSNLAWYQQKQGKSPQ |
| | GNIYPSDSATHYNQKFKDKATLT | LLVYGATNLADGVPSRFSGSGS |
| | VDKSSSTAYMHLSSLTSEDSAVY | GTQYSLKINSLQSEDFGSYYCQ |
| | YCARGLLQYFDVWGTGTSVTVS | HFWGIPWTFGGGTKLEIKRAD |
| | SAKTTPPSVYPLAPGSAAQTNSM | AAPTVSIFPPSSEQLTSGGASVV |
| | VTLGCLVKGYFPEPVTVTWNSG | CFLNNFYPRDINVKWKIDGSER |
| | SLSSGVHTFPAVLQSDLYTLSSSV | QNGVLNSWTDQDSKDSTYSMS |
| | TVPSSTWPSQTVTCNVAHPASST | STLTLTKDEYERHNSYTCEATH |
| | KVDKKIVPRDCGCKPCICTVPEV | KTSTSP |
| | SSVFIFPPKPKDVLTITLTPKVTCV | SEQ ID NO: 45 (kappa) |
| | VVDISKDDQ | CDR1 (SEQ ID NO: 46)- |
| | SEQ ID NO: 41 (IgG1) | ENIYSN |
| | CDR1 (SEQ ID NO: 42)- | CDR2 (SEQ ID NO: 47)- |
| | GYTFTSYW | GAT |
| | CDR2 (SEQ ID NO: 43)- | CDR3 (SEQ ID NO: 48)- |
| | IYPSDSAT | HFWGIP |
| | CDR3 (SEQ ID NO: 44)- | |
| | AR | |
| 4H5 | MGWSWIFLFLLSGTAGVHCQIQL | MVSTPQFLVFLLFWIPASRGDIL |
| | QQSGPELVKPGASVKISCKASGY | LTQSPAILSVSPGERVSFSCRAS |
| | TFTDYYINWVKQRPGQGLEWIG | QYIGTSIHWYQQRTNGSPRLLI |
| | WIYPGGDNTKYNEKFKDKATLT | KYASESISGIPSRFSGSGSGTDF |
| | VDTSSSTTYMQLSSLTSEDSAVY | TLSINSVESEDVADYYCQQSNR |
| | FCARFEDKYSSNYWFAYWGQGT | WPFTFGSGTKLEIKRADAAPTV |
| | LVTVSAAKTTPPSVYPLAPGSAA | SIFPPSSEQLTSGGASVVCFLNN |
| | QTNSMVTLGCLVKGYFPEPVTV | FYPRDINVKWKIDGSERQNGV |
| | TWNSGSLSSGVHTFPAVLQSDLY | LNSWTDQDSKDSTYSMSSTLT |
| | TLSSSVTVPSSTWPSQTVTCNVA | LTKDEYERHNSYTCEATHKTST |
| | HPASSTKVDKKIVPRDCGCKPCI | SP |
| | CTVPEVSSVFIFPPKPKDVLTITLT | SEQ ID NO: 53 (kappa) |
| | PKVTCVVVDISKDDQ | CDR1 (SEQ ID NO: 54)- |
| | SEQ ID NO: 49 (IgG1) | QYIGTS |
| | CDR1 (SEQ ID NO: 50)- | CDR2 (SEQ ID NO: 55)- |
| | GYTFTDYY | YAS |
| | CDR2 (SEQ ID NO: 51)- | CDR3 (SEQ ID NO: 56)- |
| | YPGGDNT | QQSNRWP |
| | CDR3 (SEQ ID NO: 52)- | |
| | AR | |
| 5B3 | MAVLVLFLCLVAFPNCVLSQVQ | MRVLAELLGLLLFCFLGVRCDI |
| | LKESGPGLVAPSQSLSITCTVSGF | QMNQSPSSLSASLGDTITITCHA |
| | SLTSYGIDWVRQPPGKGLEWLG | SQNINFWLNWYQQKPGNIPKLL |
| | VIWGGGNTNYNSALMSRLTISKD | IYKASNLHTGVPSRFSGSGSGT |
| | NSKSQVFLKMNSLQTDDTAMYY | GFTLTISSLQPEDIATYYCQQGQ |
| | CAKANWDSYAMDYWGQGTSVT | SYPLTFGGGTKVEIKRADAAPT |
| | VSSAKTTPPSVYPLAPGSAAQTN | VSIFPPSSEQLTSGGASVVCFLN |
| | SMVTLGCLVKGYFPEPVTVTWN | NFYPRDINVKWKIDGSERQNG |
| | SGSLSSGVHTFPAVLQSDLYTLSS | VLNSWTDQDSKDSTYSMSSTL |
| | SVTVPSSTWPSQTVTCNVAHPAS | TLTKDEYERHNSYTCEATHKTS |
| | STKVDKKIVPRDCGCKPCICTVP | TSP |
| | EVSSVFIFPPKPKDVLTITLTPKVT | SEQ ID NO: 61 (kappa) |
| | CVVVDISKDDQ | CDR1 (SEQ ID NO: 62)- |
| | SEQ ID NO: 57 (IgG1) | QNINFWL |
| | CDR1 (SEQ ID NO: 58)- | CDR2 (SEQ ID NO: 63)- |
| | GFSLTSYG | KAS |
| | CDR2 (SEQ ID NO: 59)- | CDR3 (SEQ ID NO: 64)- |
| | IWGGGNT | QQGQSYP |
| | CDR3 (SEQ ID NO: 60)- | |
| | AK | |
| 5E6 | MEWSWVSLFFLSVTTGVHSQVQ | MVFTPQILGLMLFWISASRGDI |
| | LQQSAAELVKPGASVKISCKVSG | VLTQSPATLSVTPGDSVSLSCR |
| | QSFTDHTIHWMKQRPEQGLEWI | ASQSISKYLHWYQQKSHESPRL |
| | GYIFPRDGFTEYNERFKGKATLT | LIKYVSQSISGIPSRFSGSGSGTD |
| | ADKSSSTAYMHLNSLTSEDSAVY | FTLSINSVETEDFGIYFCQQSNS |
| | FCAGNWDALDYWGPGTSVTVSS | WPLTFGAGTKLELKRADAAPT |
| | AKTTPPSVYPLAPGSAAQTNSMV | VSIFPPSSEQLTSGGASVVCFLN |
| | TLGCLVKGYFPEPVTVTWNSGSL | NFYPRDINVKWKIDGSERQNG |
| | SSGVHTFPAVLQSDLYTLSSSVT | VLNSWTDQDSKDSTYSMSSTL |
| | VPSSTWPSQTVTCNVAHPASSTK | TLTKDEYERHNSYTCEATHKTS |
| | VDKKIVPRDCGCKPCICTVPEVS | TSP |
| | SVFIFPPKPKDVLTITLTPKVTCV | SEQ ID NO: 69 (kappa) |
| | VVDISKDDQ | CDR1 (SEQ ID NO: 70)- |
| | SEQ ID NO: 65 (IgG1) | QSISKY |
| | CDR1 (SEQ ID NO: 66)- | CDR2 (SEQ ID NO: 71)- |
| | QSFTDHT | YVS |
| | CDR2 (SEQ ID NO: 67)- | CDR3 (SEQ ID NO: 72)- |
| | IFPRDGFT | QQSNSWP |
| | CDR3 (SEQ ID NO: 68)- | |
| | A | |
| 5E11 | MAVLALLLCLVTFPSCVLSQVQL | MMSSAQFLGLLLLCFQGTRCDI |
| | KESGPGLVAPSQSLSITCTVSGFS | QMTQTTSSLSASLGDRVTITCR |
| | LSSYGVNWVRQPPGKGLEWLGV | ASQDISNFLNWYQQKPDGPVK |
| | IWGDGSTNYHSALISRLSISKDNS | LLIYYTSRLHSGVPSRFSGSGSG |
| | KSQVFLKLNSLQTDDTATYYCA | TDYSLTISNLEQEDIATYFCQQ |
| | KPNWDYYAMEYWGQGTSVTVS | GNTLPWTFGGGTKLEIKRADA |
| | SAKTTPPSVYPLAPGSAAQTNSM | APTVSIFPPSSEQLTSGGASVVC |
| | VTLGCLVKGYFPEPVTVTWNSG | FLNNFYPRDINVKWKIDGSERQ |
| | SLSSGVHTFPAVLQSDLYTLSSSV | NGVLNSWTDQDSKDSTYSMSS |
| | TVPSSTWPSQTVTCNVAHPASST | TLTLTKDEYERHNSYTCEATH |
| | KVDKKIVPRDCGCKPCICTVPEV | KTSTSP |
| | SSVFIFPPKPKDVLTITLTPKVTCV | SEQ ID NO: 77 (kappa) |
| | VVDISKDDQ | CDR1 (SEQ ID NO: 78)- |
| | SEQ ID NO: 73 (IgG1) | QDISNF |
| | CDR1 (SEQ ID NO: 74)- | CDR2 (SEQ ID NO: 79)- |
| | GFSLSSYG | YTS |
| | CDR2 (SEQ ID NO: 75)- | CDR3 (SEQ ID NO: 80)- |
| | IWGDGST | QQGNTLP |
| | CDR3 (SEQ ID NO: 76)- | |
| | AK | |
| 5H12 | MAVLALLLCLVTFPSCVLSQVQL | MGFKMEFHTQVFVFVFLWLSG |
| | KESGPGLVAPSQSLSITCTVSGFS | VDGGIVMTQSQKFMSSTVGDR |
| | LSSYGVNWVRQPPGKGLEWLGV | VSITCKASQNVGAAVIWYQQK |
| | IWGDGSTNYHSALISRLSISKDNS | PGQSPKLLIYSASNRYTGVLDR |
| | KSQVFLKLNSLQTDDTATYYCA | FTGSGSGTDFTLTISNMQSEDL |
| | KPNWDYYAMEYWGQGTSVTVS | ADYFCQQYSSYPWTFGGGTKL |
| | SAKTTPPSVYPLAPGSAAQTNSM | EIKRADAAPTVSIFPPSSEQLTS |
| | VTLGCLVKGYFPEPVTVTWNSG | GGASVVCFLNNFYPRDINVKW |
| | SLSSGVHTFPAVLQSDLYTLSSSV | KIDGSERQNGVLNSWTDQDSK |
| | TVPSSTWPSQTVTCNVAHPASST | DSTYSMSSTLTLTKDEYERHNS |
| | KVDKKIVPRDCGCKPCICTVPEV | YTCEATHKTSTSP |
| | SSVFIFPPKPKDVLTITLTPKVTCV | SEQ ID NO: 85 (kappa) |
| | VVDISKDDQ | CDR1 (SEQ ID NO: 86)- |
| | SEQ ID NO: 81 (IgG1) | QNVGAA |
| | CDR1 (SEQ ID NO: 82)- | CDR2 (SEQ ID NO: 87)- |
| | SGFSLSSYG | SAS |
| | CDR2 (SEQ ID NO: 83)- | CDR3 (SEQ ID NO: 88)- |
| | IWGDGST | QQYSSYP |
| | CDR3 (SEQ ID NO: 84)- | |
| | AK | |
| 6A6 | MGWSWIFLFLLSGTAGVHCQVQ | MGFKMEFHTQVFVFVFLWLSG |
| | LQQSGPELVKPGASVKISCKASG | VDGDIVMTQSQKFMSTTVGDR |
| | YSFTSSYIHWVKQRPGQGLEWIG | VSITCKASQNVGTAVAWYQQK |
| | WIYPGSGNTKYNERFKGKATLT | PGQSPKLLIYSLSNRYTGVPDR |
| | ADTSSSTAYMHLSSLTSEDSAVY | FTGSGSGTDFTLTISNMQSEDL |
| | YCARNYGTSYGWYFDVWGTGT | TDYFCQQYSSYPLTFGAGTKLE |
| | TVTVSSAKTTPPSVYPLAPGCGD | LKRADAAPTVSIFPPSSEQLTSG |
| | TTGSSVTLGCLVKGYFPESVTVT | GASVVCFLNNFYPRDINVKWKI |
| | WNSGSLSSSVHTFPALLQSGLYT | DGSERQNGVLNSWTDQDSKDS |
| | MSSSVTVPSSTWPSQTVTCSVAH | TYSMSSTLTLTKDEYERHNSYT |
| | PASSTTVDKKLEPSGPISTINPCPP | CEATHKTSTSP |
| | CKECHKCPAPNLEGGPSVFIFPPN | SEQ ID NO: 93 (kappa) |
| | IKDVLMISLTPKVTCVVVDVSED | CDR1 (SEQ ID NO: 94)- |
| | DQ | QNVGTA |
| | SEQ ID NO: 89 (IgG2b) | CDR2 (SEQ ID NO: 95)- |
| | CDR1 (SEQ ID NO: 90)- | SLS |
| | ASGYSFTSSY | CDR3 (SEQ ID NO: 96)- |
| | CDR2 (SEQ ID NO: 91)- | QQYSSYP |
| | IYPGSGNT | |
| | CDR3 (SEQ ID NO: 92)- | |
| | AR | |
| 6A11 | MGWSWIFLFLLSGTAGVHCQIQL | MVSTPQFLVFLLFWIPASRGDIL |
| | QQSGPELVKPGASVKISCKASGY | LTQSPAILSVSPGERVSFSCRAS |
| | TFTDYYINWVKQRPGQGLEWIG | QYIGTSIHWYQQRTNGSPRLLI |
| | WIYPGSGNTKYNEKFKGKATLT | KYASESISGIPSRFSGSGSGTDF |
| | VDTSSSTAYMQLSSLTSEDSAVY | TLSINSVESEDIADYYCQQSNS |
| | FCARFEDKHSSNYWFAYWGQGT | WPFTFGSGTKLEIKRADAAPTV |
| | LVTVSAAKTTPPSVYPLAPGSAA | SIFPPSSEQLTSGGASVVCFLNN |
| | QTNSMVTLGCLVKGYFPEPVTV | FYPRDINVKWKIDGSERQNGV |
| | TWNSGSLSSGVHTFPAVLQSDLY | LNSWTDQDSKDSTYSMSSTLT |
| | TLSSSVTVPSSTWPSQTVTCNVA | LTKDEYERHNSYTCEATHKTST |
| | HPASSTKVDKKIVPRDCGCKPCI | S |
| | CTVPEVSSVFIFPPKPKDVLTITLT | SEQ ID NO: 101 (kappa) |
| | PKVTCVVVDISKDDQ | CDR1 (SEQ ID NO: 102)- |
| | SEQ ID NO: 97 (IgG1) | QYIGTS |
| | CDR1 (SEQ ID NO: 89)- | CDR2 (SEQ ID NO: 103)- |
| | SGYTFTDYY | YAS |
| | CDR2 (SEQ ID NO: 99)- | CDR3 (SEQ ID NO: 104)- |
| | IYPGSGNT | QQSNSWP |
| | CDR3 (SEQ ID NO; 100)- | |
| | AR | |
| 6B7 | MGWLWNLLFLMAAAQSAQAQI | MTMLSLAPLLSLLLLCVSDSRA |
| | QLVQSGPELKKPGETVKISCKAS | ETTVTQSPASLSVATGEKVTIR |
| | GYTFTNYGLSWVKQAPGKGLK | CITSTDIDDDINWYQQKPGEPP |
| | WMGWINTYSGVTTYADDFKGRF | KFLISEGNTLRPGVPSRFSSSGY |
| | AFSLETSASTAYLQINNLKSEDTA | GTDFVFTIENTLSEDVADYYCL |
| | TYFCARRSYDYSHYYAMDYWG | QSNNMPYTFGGGTKLEIKRAD |
| | QGTSVTVSSAKTTPPSVYPLAPG | AAPTVSIFPPSSEQLTSGGASVV |
| | SAAQTNSMVTLGCLVKGYFPEP | CFLNNFYPRDINVKWKIDGSER |
| | VTVTWNSGSLSSGVHTFPAVLQS | QNGVLNSWTDQDSKDSTYSMS |
| | DLYTLSSSVTVPSSTWPSQTVTC | STLTLTKDEYERHNSYTCEATH |
| | NVAHPASSTKVDKKIVPRDCGC | KTSTSP |
| | KPCICTVPEVSSVFIFPPKPKDVL | SEQ ID NO: 109 (kappa) |
| | TITLTPKVTCVVVDISKDDQ | CDR1 (SEQ ID NO: 110)- |
| | SEQ ID NO: 105 (IgG1) | TDIDDD |
| | CDR1 (SEQ ID NO: 106)- | CDR2 (SEQ ID NO: 111)- |
| | GYTFTNYG | +EGN |
| | CDR2 (SEQ ID NO: 107)- | CDR3 (SEQ ID NO; 112)- |
| | INTYSGVT | QSNNMP |
| | CDR3 (SEQ ID NO; 108)- | |
| | AR | |

TABLE 2
(LILRB2 Antibodies)

| | Heavy chain variable region | Light chain variable region |
|---|---|---|
| Clone | amino acid sequence | amino acid sequence |
| 1A8 | MNFGLSLIFLALILKGVQCEVQL | METDTLLLWVLLLWVPGSTGD |
| | VESGGDLVKPGESLKLSCAASGF | IVLTQSPASLAVSLGQRATISCR |
| | SFSSYDMSWVRQTPDKRLEWVT | ASESVDKSGISFLHWYQQKPRQ |
| | TISSGGDYTYFPDILKGRFTISRD | PPKLLIYRASNLESGIPGRESGS |
| | NAKHTLYLQLSSLKSEDTAMYY | GSRTDFTLSINPVEPDDVATYY |
| | CARWDSNFPHWYFDVWGTGTT | CQQSNKDPFTFGGGTKLEIKRA |
| | VTVSSAKTTPPSVYPLAPGSAAQ | DAAPTVSIFPPSSEQLTSGGASV |
| | TNSMVTLGCLVKGYFPEPVTVT | VCFLNNFYPRDINVKWKIDGSE |
| | WNSGSLSSGVHTFPAVLQSDLYT | RQNGVLNSWTDQDSKDSTYS |
| | LSSSVTVPSSTWPSQTVTCNVAH | MSSTLTLTKDEYERHNSYTCEA |
| | PASSTKVDKKIVPRDCGCKPCIC | THKTSTSP |
| | TVPEVSSVFIFPPKPKDVLTITLTP | SEQ ID NO: 117 (kappa) |
| | KVTCVVVDISKDD | CDR1 (SEQ ID NO: 118)- |
| | SEQ ID NO: 113 (IgG1) | ESVDKSGISF |
| | CDR1 (SEQ ID NO: 114)- | CDR2 (SEQ ID NO: 119)- |
| | GFSFSSYD | RAS |
| | CDR2 (SEQ ID NO: 115)- | CDR3 (SEQ ID NO: 120)- |
| | TISSGGDYT | QQSNKDP |
| | CDR3 (SEQ ID NO: 116)- | |
| | AR | |
| 1C8 | MNLGLSLIFLVLVLKGVQCEVKL | MGIKMESQIQAFVFVFL |
| | VESGGGLVQPGGSLKLSCAASGF | WLSGVDGDIVMTQSHKFMSTS |
| | TFSDYYMYWVRQTPEKRLEWV | VGDRVSITCKASQDVSTAVAW |
| | AYISNGGGNTYYPDTVKGRFTIS | SQQKPGQSPKLLVYWASARHT |
| | RDNAKNTLYLQMSRLKSEDTAM | GVPDRFTGSGSGADYTLTISSV |
| | YYCARQLEDWYFDVWGTGTTV | QAEDLALYYCQQHFSTPLTFG |
| | TVSSAKTTPPSVYPLAPGSAAQT | AGTKLELKRADAAPTVSIFPPSS |
| | NSMVTLGCLVKGYFPEPVTVTW | EQLTSGGASVVCFLNNFYPRDI |
| | NSGSLSSGVHTFPAVLQSDLYTL | NVKWKIDGSERQNGVLNSWT |
| | SSSVTVPSSTWPSQTVTCNVAHP | DQDSKDSTYSMSSTLTLTKDEY |
| | ASSTKVDKKIVPRDCGCKPCICT | ERHNSYTCEATHKTSTSP |
| | VPEVSSVFIFPPKPKDVLTITLTPK | SEQ ID NO: 125 (kappa) |
| | VTCVVVDISKDD | CDR1 (SEQ ID NO: 126)- |
| | SEQ ID NO: 121 (IgG1) | QDVSTA |
| | CDR1 (SEQ ID NO: 122)- | CDR2 (SEQ ID NO: 127)- |
| | GFTFSDYY | WAS |
| | CDR2 (SEQ ID NO: 123)- | CDR3 (SEQ ID NO: 128)- |
| | SNGGGNT | CQQHFSTP |
| | CDR3 (SEQ ID NO: 124) | |
| | AR | |
| 1G3 | MNFGLSLIFLALILKGVQCEVQL | METDTLLLWVLLLWVP |
| | VESGGDLVKPGGSLKLSCAASGF | GSTGDIVLTQSPASLAVSLGQR |
| | TFSSYDMSWVRQTPDKRLEWVA | ATISCRASESVDNNGISFMHWY |
| | TISSGGSYTFYPDSVKGRFTISRD | QQKPGQSPKLLIYRASNLESGIP |
| | NAKNTLSLQMSSLKSEDTAIYYC | ARFSGSGSRTDFTLTINPVETDD |
| | ARWDSNYLRWFFDVWGTGTTV | VATYYCQQSNDDPFTFGGGTK |
| | TVSSAKTTPPSVYPLAPGSAAQT | LEIKRADAAPTVSIFPPSSEQLT |
| | NSMVTLGCLVKGYFPEPVTVTW | SGGASVVCFLNNFYPRDINVK |
| | NSGSLSSGVHTFPAVLQSDLYTL | WKIDGSERQNGVLNSWTDQDS |
| | SSSVTVPSSTWPSQTVTCNVAHP | KDSTYSMSSTLTLTKDEYERHN |
| | ASSTKVDKKIVPRDCGCKPCICT | SYTCEATHKTSTSP |
| | VPEVSSVFIFPPKPKDVLTITLTPK | SEQ ID NO: 133 (kappa) |
| | VTCVVVDISKDD | CDR1 (SEQ ID NO: 134)- |
| | SEQ ID NO: 129 (IgG1) | ESVDNNGISF |
| | CDR1 (SEQ ID NO: 130)- | CDR2 (SEQ ID NO: 135)- |
| | GFTFSSYD | RAS |
| | CDR2 (SEQ ID NO: 131)- | CDR3 (SEQ ID NO: 136)- |
| | ISSGGSYT | QQSNDDP |
| | CDR3 (SEQ ID NO: 132)- | |
| | AR | |
| 2C5 | MNFGLSLIFLALILKGVQCEVQL | MHHTSMGIKMESQIQVFVFVFL |
| | VESGGDLVKPGGSLKLSCAASGF | WLSGVDGDIVMTQSHKFKSTS |
| | TFSSYDMSWVRQTPDKRLEWVA | VGDRVNITCKASQDVSIAVAW |
| | TINSGGSYSYYPDSVKGRFTMSR | YQQKPGQSPKLLIYAASYRYTG |
| | DNAKNTLYLQMSSLKSEDTAMY | VPDRFTGSGSGTDFTFTISSVQA |
| | YCARPSYGNSFDYWGQGTSLTV | EDLAVYYCQQHYSIPWTFGGG |
| | SSAKTTPPSVYPLAPGSAAQTNS | TKLEIKRADAAPTVSIFPPSSEQ |
| | MVTLGCLVKGYFPEPVTVTWNS | LTSGGASVVCFLNNFYPRDINV |
| | GSLSSGVHTFPAVLQSDLYTLSSS | KWKIDGSERQNGVLNSWTDQ |
| | VTVPSSTWPSQTVTCNVAHPASS | DSKDSTYSMSSTLTLTKDEYER |
| | TKVDKKIVPRDCGCKPCICTVPE | HNSYTCEATHKTSTSP |
| | VSSVFIFPPKPKDVLTITLTPKVTC | SEQ ID NO: 141 (kappa) |
| | VVVDISKDD | CDR1 (SEQ ID NO: 142)- |
| | SEQ ID NO: 137 (IgG1) | QDVSIA |
| | CDR1 (SEQ ID NO: 138)- | CDR2 (SEQ ID NO: 143)- |
| | GFTFSSYD | AAS |
| | CDR2 (SEQ ID NO: 139)- | CDR3 (SEQ ID NO: 144)- |
| | INSGGSYS | QQHYSIP |
| | CDR3 (SEQ ID NO: 140)- | |
| | AR | |
| 2F10 | MGWSWIFLFLLSGTAGVLSEVQL | MMSSAQFLGLLLLCFQGSRCDI |
| | QQSGPELVKPGASVKISCKASGY | QMTQTTSSLSASLGDRVTISCS |
| | TFTDDYMNWVKQSHGKSLEWIG | ASQGISNYLNWYQQKPDGTVK |
| | DINPTNGDTNYNQKFKGKATLT | LLIYYTSSLYSGVPSRFSGSGSG |
| | VDKSSSTAYMELRGLTSEDSAVY | TDYSLTISNLEPEDIATYYCQQ |
| | YCARGSSPFTYWGQGTLVTVSA | YSKLPWTFGGGTKLEIKRADA |
| | AKTTPPSVYPLAPGCGDTTGSSV | APTVSIFPPSSEQLTSGGASVVC |
| | TLGCLVKGYFPESVTVTWNSGSL | FLNNFYPRDINVKWKIDGSERQ |
| | SSSVHTFPALLQSGLYTMSSSVT | NGVLNSWTDQDSKDSTYSMSS |
| | VPSSTWPSQTVTCSVAHPASSTT | TLTLTKDEYERHNSYTCEATH |
| | VDKKLEPSGPISTINPCPPCKECH | KTSTSP |
| | KCPAPNLEGGPSVFIFPPNIKDVL | SEQ ID NO: 149 (kappa) |
| | MISLTPKVTCVVVDVSEDD | CDR1 (SEQ ID NO: 150)- |
| | SEQ ID NO: 145 (IgG2b) | QGISNY |
| | CDR1 (SEQ ID NO: 146)- | CDR2 (SEQ ID NO: 151)- |
| | GYTFTDDY | YTS |
| | CDR2 (SEQ ID NO: 147)- | CDR3 (SEQ ID NO: 152)- |
| | INPTNGDT | QQYSKLP |
| | CDR3 (SEQ ID NO: 148)- | |
| | AR | |
| 2G9 | MNLGLSLIFLVLVLKGVQCEVKL | MESQTQVLMFLLLWVSGACA |
| | VESGGGLVQPGGSLKLSCAASGF | DIVMTQSPSSLAMSVGQKVTM |
| | TFSDYYMYWVRQTPEKRLEWV | NCKSSQSLLISSNQKNYLAWY |
| | AYISNGGGNTYYPDTVKGRFTIS | QQKPGQSPKLLIYFASTRESGV |
| | RDNAKNTLYLQMSRLKSEDTAM | PDRFIGSGSGTDFTLTISSVQAE |
| | YYCARNRDDWYFDVWGTGTTV | DLADYFCQQHYSTPLTFGAGT |
| | TVSSAKTTAPSVYPLAPVCGGTT | KLELKRADAAPTVSIFPPSSEQL |
| | GSSVTLGCLVKGYFPEPVTLTWN | TSGGASVVCFLNNFYPRDINVK |
| | SGSLSSGVHTFPALLQSGLYTLSS | WKIDGSERQNGVLNSWTDQDS |
| | SVTVTSNTWPSQTITCNVAHPAS | KDSTYSMSSTLTLTKDEYERHN |
| | STKVDKKIEPRVPITQNPCPPLKE | SYTCEATHKTSTSP |
| | CPPCAAPDLLGGPSVFIFPP | SEQ ID NO: 157 (kappa) |
| | SEQ ID NO: 153 (IgG2c) | CDR1 (SEQ ID NO: 158)- |
| | CDR1 (SEQ ID NO: 154)- | QSLLISSNQKNY |
| | GFTFSDYY | CDR2 (SEQ ID NO: 159)- |
| | CDR2 (SEQ ID NO: 155)- | FAS |
| | SNGGGNT | CDR3 (SEQ ID NO: 160)- |
| | CDR3 (SEQ ID NO: 156)- | QQHYSTP |
| | AR | |
| 3B3 | MGWSCIILILVAAATGVHSQVQL | MHQTSMGIKMESQTLVFISILL |
| | QQPGAELVKPGASVKMSCKASD | WLYGADGNIVMTQSPKSMSMS |
| | YTFTSYWITWVKKRPGQGLEWI | VGERVTLSCKASENVGTYVSW |
| | GDIYPGSDTTNYNEKFKNKATLT | YQQKPEQSPKLLIYGASNRYTG |
| | VDTSSSTAHMQLSSLTSEDSAVY | VPDRFTGSGSATDFTLTISSVQA |
| | YCTRPLYQGISPWFAYWGQGTL | EDLADYHCGQSYSYPFTFGSGT |
| | VTVSAAKTTPPSVYPLAPGSAAQ | KLEIKRADAAPTVSIFPPSSEQL |
| | TNSMVTLGCLVKGYFPEPVTVT | TSGGASVVCFLNNFYPRDINVK |
| | WNSGSLSSGVHTFPAVLQSDLYT | WKIDGSERQNGVLNSWTDQDS |
| | LSSSVTVPSSTWPSQTVTCNVAH | KDSTYSMSSTLTLTKDEYERHN |
| | PASSTKVDKKIVPRDCGCKPCIC | SYTCEATHKTSTSP |
| | TVPEVSSVFIFPPKPKDVLTITLTP | SEQ ID NO: 165 (kappa) |
| | KVTCVVVDISKDD | CDR1 (SEQ ID NO: 166)- |
| | SEQ ID NO: 161 (IgG1) | ENVGTY |
| | CDR1 (SEQ ID NO: 162)- | CDR2 (SEQ ID NO: 167)- |
| | DYTFTSYW | GAS |
| | CDR2 (SEQ ID NO: 163)- | CDR3 (SEQ ID NO: 168)- |
| | IYPGSDTT | GQSYSYP |
| | CDR3 (SEQ ID NO: 164)- | |
| | TR | |
| 6B3 | MGWSWIFLFLLSGTAGVLSEVQL | MMSSAQFLGLLLLCFQGTRCDI |
| | QESGPELLKPGASMKISCKASGY | QMTQTTSSLSASLGDRVTINCR |
| | TFTDDYMNWVKQSHGKSLEWIG | ASQDIRNYLSWYQQKPDGTVK |
| | DINPNNGGTSYNQKFKGKATLT | LLIYYTSRLHSGVPSRFSGSGSG |
| | VDKSSSTAYMELRSLTSEDSAVY | RDYSLTISNLEQEDIATYFCQQS |
| | YCARGSPWFAYWGQGTLVTVSA | NTLPWTFGGGTKLEITRADAAP |
| | AKTTPPSVYPLAPGSAAQTNSMV | TVSIFPPSSEQLTSGGASVVCFL |
| | TLGCLVKGYFPEPVTVTWNSGSL | NNFYPRDINVKWKIDGSERQN |
| | S | GVLNSWTDQDSKDSTYSMSST |
| | SEQ ID NO: 169 (IgG1) | LTLTKDEYERHNSYTCEATHK |
| | CDR1 (SEQ ID NO: 170)- | TSTSP |
| | GYTFTDDY | SEQ ID NO: 173 (kappa) |
| | CDR2 (SEQ ID NO: 171)- | CDR1 (SEQ ID NO: 174)- |
| | INPNNGGT | QDIRNY |
| | CDR3 (SEQ ID NO: 172)- | CDR2 (SEQ ID NO: 175)- |
| | AR | YTS |
| | | CDR3 (SEQ ID NO: 176)- |
| | | QQSNTLP |
| 8D2 | MGWLWNLLFLMAAAQSAQAQI | MSVPTQVLGLLLLWLTAARCDI |
| | QLVQSGPELKKPGETVKISCKAS | QMTQSPASLSVSVGETVTITCR |
| | GYTFTTYGMSWVKQAPGKGLK | ASENIYSNLAWYQQKQGKSPQ |
| | WMGWINTYSGAPAYVDDFKGR | LLVYAATNLADGVPSRFSGSGS |
| | FAFSLETSASTAYLQINNLKNEDT | GTQYSLKINSLQSEDFGSYYCQ |
| | ATYFCARHFYSGSSYWYFDVWG | HFWGTPRTFGGGTKLEIKRADA |
| | TGTTVTVSSAKTTPPSVYPLAPGS | APTVSIFPPSSEQLTSGGASVVC |
| | AAQTNSMVTLGCLVKGYFPEPV | FLNNFYPRDINVKWKIDGSERQ |
| | TVTWNSGSLSSGVHTFPAVLQSD | NGVLNSWTDQDSKDSTYSMSS |
| | LYTLSSSVTVPSSTWPSQTVTCN | TLTLTKDEYERHNSYTCEATHK |
| | VAHPASSTKVDKKIVPRDCGCKP | TSTSP |
| | CICTVPEVSSVFIFPPKPKDVLTIT | SEQ ID NO: 181 (kappa) |
| | LTPKVTCVVVDISKDD | CDR1 (SEQ ID NO: 182)- |
| | SEQ ID NO: 177 (IgG1) | ENIYSN |
| | CDR1 (SEQ ID NO: 178)- | CDR2 (SEQ ID NO: 183)- |
| | GYTFTTYG | AAT |
| | CDR2 (SEQ ID NO: 179)- | CDR3 (SEQ ID NO: 184)- |
| | NTYSGAP | QHFWGTP |
| | CDR3 (SEQ ID NO: 180)- | |
| | AR | |
| 10A8 | MNFGLSLIFLALILKGVQCEVQL | MHHTSMGIKMESQIQVFVFVFL |
| | VESGGDFVKPGGSLKLSCAASGF | WLSGVDGDIVMTQSHKFMSTS |
| | TFSSYDMSWVRQTPDKRLEWVA | IGDRVSITCKASQDVNNAVAW |
| | TIIRGDSYTYYLDSVKGRFTISRD | YQQKPGQSPKLLIYAASYRYTG |
| | NAKNTLYLQMSSLKSEDTAMYY | VPDRFTGSGSGTDFTFTISSVQA |
| | CARPSYGNSFDYWGQGTTLTVS | EDLAVYHCQQHYGIPWTFGGG |
| | SAKTTPPSVYPLAPGSAAQTNSM | TKLEIKRADAAPTVSIFPPSSEQ |
| | VTLGCLVKGYFPEPVTVTWNSG | LTSGGASVVCFLNNFYPRDINV |
| | SLSSGVHTFPAVLQSDLYTLSSSV | KWKIDGSERQNGVLNSWTDQ |
| | TVPSSTWPSQTVTCNVAHPASST | DSKDSTYSMSSTLTLTKDEYER |
| | KVDKKIVPRDCGCKPCICTVPEV | HNSYTCEATHKTSTSP |
| | SSVFIFPPKPKDVLTITLTPKVTCV | SEQ ID NO: 189 (kappa) |
| | VVDISKDD | CDR1 (SEQ ID NO: 190)- |
| | SEQ ID NO: 185 (IgG1) | QDVNNA |
| | CDR1 (SEQ ID NO: 186)- | CDR2 (SEQ ID NO: 191)- |
| | GFTFSSYD | AAS |
| | CDR2 (SEQ ID NO: 187)- | CDR3 (SEQ ID NO: 192)- |
| | IIRGDSYT | QQHYGIP |
| | CDR3 (SEQ ID NO: 188)- | |
| | AR | |
| 10C7 | MKVLSLLYLLTAIPGILSDVQLQE | MSVPTQVLGLLLLWLTGARCD |
| | SGPGLVKPSQSLSLTCSVTGYSIT | IQMTQSPASLSASVGETVTITCR |
| | SGYYWNWIRQFPGNKLEWMGYI | ASENIYSYLAWYQQKQGKSPQ |
| | SYDGSNNYNPSLKNRISITRDTSK | LLVYNAKTLAEGVPSRFSGSGS |
| | NQFFLKLNSVTTEDTATYYCARG | DTQFSLKINSLQPEDFGIFYCQH |
| | GWLLSRYWGQGTSVTVSSAKTT | FYGDLPTFGAGTKLELKRADA |
| | PPSVYPLAPGSAAQTNSMVTLGC | APTVSIFPPSSEQLTSGGASVVC |
| | LVKGYFPEPVTVTWNSGSLSSGV | FLNNFYPRDINVKWKIDGSERQ |
| | HTFPAVLQSDLYTLSSSVTVPSST | NGVLNSWTDQDSKDSTYSMSS |
| | WPSQTVTCNVAHPASSTKVDKKI | TLTLTKDEYERHNSYTCEATH |
| | VPRDCGCKPCICTVPEVSSVFIFP | KTSTSP |
| | PKPKDVLTITLTPKVTCVVVDISK | SEQ ID NO: 197 (kappa) |
| | DD | CDR1 (SEQ ID NO: 198)- |
| | SEQ ID NO: 193 (IgG1) | ENIYSY |
| | CDR1 (SEQ ID NO: 194)- | CDR2 (SEQ ID NO: 199)- |
| | GYSITSGYY | NAK |
| | CDR2 (SEQ ID NO: 195)- | CDR3 (SEQ ID NO: 200)- |
| | ISYDGSN | QHFYG |
| | CDR3 (SEQ ID NO: 196)- | |
| | AR | |
| 10D4 | MEWSWVSLFFLSVTTGVHSQVQ | MDMRTPAQFLGILLLWFPGIKC |
| | LQQSDAELVKPGASVKISCKVSG | DIKMTQSPSSMHASLGERVTIT |
| | YTFSDHTFHWMKQRPEQGLEWI | CKASQDINSYLSWFQQKPGKSP |
| | GYFYPRDGRSKYNEKFRDKATL | KTLIYHADRLVDGVPSRFSGSG |
| | TADKSSSTAYMQLNSLTSEDSAV | SGQDYSLTISSLEYEDMGIYYC |
| | YFCTCDGFDYWGQGTTLTVSSA | LQYDEFPYTFGGGTKLEIKRAD |
| | KTTPPSVYPLAPGSAAQTNSMVT | AAPTVSIFPPSSEQLTSGGASVV |
| | LGCLVKGYFPEPVTVTWNSGSLS | CFLNNFYPRDINVKWKIDGSER |
| | SGVHTFPAVLQSDLYTLSSSVTV | QNGVLNSWTDQDSKDSTYSMS |
| | PSSTWPSQTVTCNVAHPASSTKV | STLTLTKDEYERHNSYTCEATH |
| | DKKIVPRDCGCKPCICTVPEVSS | KTSTSP |
| | VFIFPPKPKDVLTITLTPKVTCVV | SEQ ID NO: 204 (kappa) |
| | VDISKDD | CDR1 (SEQ ID NO: 205)- |
| | SEQ ID NO: 201 (IgG1) | QDINSY |
| | CDR1 (SEQ ID NO: 202)- | CDR2 (SEQ ID NO: 206)- |
| | GYTFSDHT | HAD |
| | CDR2 (SEQ ID NO: 203)- | CDR3 (SEQ ID NO: 207)- |
| | FYPRDGRS | LQYDEFP |
| | CDR3 (not identified) | |
| 10F5 | MNFGLSLIFLALILKGVQCEVQL | METDTLLLWVLLLWVPGSTGD |
| | VESGGDLVKPGGSLKLSCAASGF | IVLTQSPASLAVSLGQRATISCR |
| | TFSSYDMSWVRQTPDKRLEWVA | ASESVDNNGISFMHWYQQKPG |
| | TISSGASYTYYPDSVKGRFTISRD | QSPKLLISRASNLESGIPARFSG |
| | NAKNTLYLQMSSLKSEDTAIYYC | SGSRTDFTLTINPVETDDVATY |
| | ARWDSKYLRWYFDVWGTGTTV | YCQQSNEDPFTFGGGTKLEIKR |
| | TVSSAKTTPPSVYPLAPGSAAQT | ADAAPTVSIFPPSSEQLTSGGAS |
| | NSMVTLGCLVKGYFPEPVTVTW | VVCFLNNFYPRDINVKWKIDGS |
| | NSGSLSSGVHTFPAVLQSDLYTL | ERQNGVLNSWTDQDSKDSTYS |
| | SSSVTVPSSTWPSQTVTCNVAHP | MSSTLTLTKDEYERHNSYTCEA |
| | ASSTKVDKKIVPRDCGCKPCICT | THKTSTSP |
| | VPEVSSVFIFPPKPKDVLTITLTPK | SEQ ID NO: 212 (kappa) |
| | VTCVVVDISKDD | CDR1 (SEQ ID NO: 213)- |
| | SEQ ID NO: 208 (IgG1) | ESVDNNGISF |
| | CDR1 (SEQ ID NO: 209)- | CDR2 (SEQ ID NO: 214)- |
| | GFTFSSYD | RAS |
| | CDR2 (SEQ ID NO: 210)- | CDR3 (SEQ ID NO: 215)- |
| | TISSGASYT | QQSNEDP |
| | CDR3 (SEQ ID NO: 211)- | |
| | AR | |

In some embodiments, the anti-LILRB1 antibodies in the present disclosure include a heavy chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%9, 97%, 98%, 99%, or 100%) identical to SEQ ID NO:1 and a light chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:5.

In some embodiments, the anti-LILRB antibodies include a vhCDR1 comprising SEQ ID NO:2, a vhCDR2 comprising SEQ ID NO:3, a vhCDR3 comprising SEQ ID NO:4, a vlCDR1 comprising SEQ ID NO:6, a vlCDR2 comprising SEQ ID NO:7, and a vlCDR3 comprising SEQ ID NO:8. In some embodiments, one or more of such 6 CDRs have from 1, 2, 3, 4 or 5 amino acid modifications. In further embodiments, a single CDR contains 1 or 2 amino acid substitutions, and the modified anti-LILRB antibodies retain binding to human LILRB. As will be appreciated, any mentions of LILRB and/or anti-LILRB antibodies herein encompass any LILRB variants, including LILRB1 and/or LILRB2.

In some embodiments, the anti-LILRB antibodies in the present disclosure include a heavy chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%9, 97%, 98%, 99%, or 100%) identical to SEQ ID NO:9 and a light chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:13.

In some embodiments, the anti-LILRB antibodies include a vhCDR1 comprising SEQ ID NO:10, a vhCDR2 comprising SEQ ID NO:11, a vhCDR3 comprising SEQ ID NO:12, a vlCDR1 comprising SEQ ID NO: 14, a vlCDR2 comprising SEQ ID NO:15, and a vlCDR3 comprising SEQ ID NO:16. In further embodiments, a single CDR contains 1 or 2 amino acid substitutions, and the modified anti-LILRB antibodies retain binding to human LILRB.

In some embodiments, the anti-LILRB antibodies in the present disclosure include a heavy chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%9, 97%, 98%, 99%, or 100%) identical to SEQ ID NO:17 and a light chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:21.

In some embodiments, the anti-LILRB antibodies include a vhCDR1 comprising SEQ ID NO:18, a vhCDR2 comprising SEQ ID NO:19, a vhCDR3 comprising SEQ ID NO:20, a vlCDR1 comprising SEQ ID NO:22, a vlCDR2 comprising SEQ ID NO:23, and a vlCDR3 comprising SEQ ID NO:24. In some embodiments, one or more of such 6 CDRs have from 1, 2, 3, 4 or 5 amino acid modifications. In further embodiments, a single CDR contains 1 or 2 amino acid substitutions, and the modified anti-LILRB antibodies retain binding to human LILRB.

In some embodiments, the anti-LILRB antibodies in the present disclosure include a heavy chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%9, 97%, 98%, 99%, or 100%) identical to SEQ ID NO:25 and a light chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:29.

In some embodiments, the anti-LILRB antibodies include a vhCDR1 comprising SEQ ID NO:26, a vhCDR2 comprising SEQ ID NO:27, a vhCDR3 comprising SEQ ID NO:28, a vlCDR1 comprising SEQ ID NO:30, a vlCDR2 comprising SEQ ID NO:31, and a vlCDR3 comprising SEQ ID NO:32. In some embodiments, one or more of such 6 CDRs have from 1, 2, 3, 4 or 5 amino acid modifications. In further embodiments, a single CDR contains 1 or 2 amino acid substitutions, and the modified anti-LILRB antibodies retain binding to human LILRB.

In some embodiments, the anti-LILRB antibodies in the present disclosure include a heavy chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%9, 97%, 98%, 99%, or 100%) identical to SEQ ID NO:33 and a light chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:37.

In some embodiments, the anti-LILRB antibodies include a vhCDR1 comprising SEQ ID NO:34, a vhCDR2 comprising SEQ ID NO:35, a vhCDR3 comprising SEQ ID NO:36, a vlCDR1 comprising SEQ ID NO:38, a vlCDR2 comprising SEQ ID NO:39, and a vlCDR3 comprising SEQ ID NO:40. In some embodiments, one or more of such 6 CDRs have from 1, 2, 3, 4 or 5 amino acid modifications. In further embodiments, a single CDR contains 1 or 2 amino acid substitutions, and the modified anti-LILRB antibodies retain binding to human LILRB.

In some embodiments, the anti-LILRB antibodies in the present disclosure include a heavy chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%9, 97%, 98%, 99%, or 100%) identical to SEQ ID NO:41 and a light chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:45.

In some embodiments, the anti-LILRB antibodies that include a vhCDR1 comprising SEQ ID NO:42, a vhCDR2 comprising SEQ ID NO:43, a vhCDR3 comprising SEQ ID NO:44, a vlCDR1 comprising SEQ ID NO:46, a vlCDR2 comprising SEQ ID NO:47, and a vlCDR3 comprising SEQ ID NO:48. In some embodiments, one or more of such 6 CDRs have from 1, 2, 3, 4 or 5 amino acid modifications. In further embodiments, a single CDR contains 1 or 2 amino acid substitutions, and the modified anti-LILRB antibodies retain binding to human LILRB.

In some embodiments, the anti-LILRB antibodies in the present disclosure include a heavy chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%9, 97%, 98%, 99%, or 100%) identical to SEQ ID NO:49 and a light chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:53.

In some embodiments, the anti-LILRB antibodies that include a vhCDR1 comprising SEQ ID NO:50, a vhCDR2 comprising SEQ ID NO:51, a vhCDR3 comprising SEQ ID NO:52, a vlCDR1 comprising SEQ ID NO:54, a vlCDR2 comprising SEQ ID NO:55, and a vlCDR3 comprising SEQ ID NO:56. In some embodiments, one or more of such 6 CDRs have from 1, 2, 3, 4 or 5 amino acid modifications. In further embodiments, a single CDR contains 1 or 2 amino acid substitutions, and the modified anti-LILRB antibodies retain binding to human LILRB.

In some embodiments, the anti-LILRB antibodies in the present disclosure include a heavy chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%9, 97%, 98%, 99%, or 100%) identical to SEQ ID NO:57 and a light chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:61.

In some embodiments, the anti-LILRB antibodies that include a vhCDR1 comprising SEQ ID NO:58, a vhCDR2 comprising SEQ ID NO:59, a vhCDR3 comprising SEQ ID NO:60, a vlCDR1 comprising SEQ ID NO:62, a vlCDR2 comprising SEQ ID NO:63, and a vlCDR3 comprising SEQ ID NO:64. In some embodiments, one or more of such 6 CDRs have from 1, 2, 3, 4 or 5 amino acid modifications. In further embodiments, a single CDR contains 1 or 2 amino acid substitutions, and the modified anti-LILRB antibodies retain binding to human LILRB.

In some embodiments, the anti-LILRB antibodies in the present disclosure include a heavy chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%9, 97%, 98%, 99%, or 100%) identical to SEQ ID NO:65 and a light chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:69.

In some embodiments, the anti-LILRB antibodies include a vhCDR1 comprising SEQ ID NO:66, a vhCDR2 comprising SEQ ID NO:67, a vhCDR3 comprising SEQ ID NO:68, a vlCDR1 comprising SEQ ID NO:70, a vlCDR2 comprising SEQ ID NO:71, and a vlCDR3 comprising SEQ ID NO:72. In some embodiments, one or more of such 6 CDRs have from 1, 2, 3, 4 or 5 amino acid modifications. In further embodiments, a single CDR contains 1 or 2 amino acid substitutions, and the modified anti-LILRB antibodies retain binding to human LILRB.

In some embodiments, the anti-LILRB antibodies in the present disclosure include a heavy chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%9, 97%, 98%, 99%, or 100%) identical to SEQ ID NO:73 and a light chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:77.

In some embodiments, the anti-LILRB antibodies include a vhCDR1 comprising SEQ ID NO:74, a vhCDR2 comprising SEQ ID NO:75, a vhCDR3 comprising SEQ ID NO:76, a vlCDR1 comprising SEQ ID NO:78, a vlCDR2 comprising SEQ ID NO:79, and a vlCDR3 comprising SEQ ID NO:80. In some embodiments, one or more of such 6 CDRs have from 1, 2, 3, 4 or 5 amino acid modifications. In further embodiments, a single CDR contains 1 or 2 amino acid substitutions, and the modified anti-LILRB antibodies retain binding to human LILRB.

In some embodiments, the anti-LILRB antibodies in the present disclosure include a heavy chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%9, 97%, 98%, 99%, or 100%) identical to SEQ ID NO:81 and a light chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:85.

In some embodiments, the anti-LILRB antibodies include a vhCDR1 comprising SEQ ID NO:82, a vhCDR2 comprising SEQ ID NO:83, a vhCDR3 comprising SEQ ID NO:84, a vlCDR1 comprising SEQ ID NO:86, a vlCDR2 comprising SEQ ID NO:87, and a vlCDR3 comprising SEQ ID NO:88. In some embodiments, one or more of such 6 CDRs have from 1, 2, 3, 4 or 5 amino acid modifications. In further embodiments, a single CDR contains 1 or 2 amino acid substitutions, and the modified anti-LILRB antibodies retain binding to human LILRB.

In some embodiments, the anti-LILRB antibodies in the present disclosure include a heavy chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%9, 97%, 98%, 99%, or 100%) identical to SEQ ID NO:89 and a light chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:93.

In some embodiments, the anti-LILRB antibodies include a vhCDR1 comprising SEQ ID NO:90, a vhCDR2 comprising SEQ ID NO:91, a vhCDR3 comprising SEQ ID NO:92, a vlCDR1 comprising SEQ ID NO:94, a vlCDR2 comprising SEQ ID NO:95, and a vlCDR3 comprising SEQ ID NO:96. In some embodiments, one or more of such 6 CDRs have from 1, 2, 3, 4 or 5 amino acid modifications. In further embodiments, a single CDR contains 1 or 2 amino acid substitutions, and the modified anti-LILRB antibodies retain binding to human LILRB.

In some embodiments, the anti-LILRB antibodies in the present disclosure include a heavy chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%9, 97%, 98%, 99%, or 100%) identical to SEQ ID NO:97 and a light chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:101.

In some embodiments, the anti-LILRB antibodies include a vhCDR1 comprising SEQ ID NO:98, a vhCDR2 comprising SEQ ID NO:99, a vhCDR3 comprising SEQ ID NO:100, a vlCDR1 comprising SEQ ID NO: 102, a vlCDR2 comprising SEQ ID NO:103, and a vlCDR3 comprising SEQ ID NO: 104. In some embodiments, one or more of such 6 CDRs have from 1, 2, 3, 4 or 5 amino acid modifications. In further embodiments, a single CDR contains 1 or 2 amino acid substitutions, and the modified anti-LILRB antibodies retain binding to human LILRB.

In some embodiments, the anti-LILRB antibodies in the present disclosure include a heavy chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%9, 97%, 98%, 99%, or 100%) identical to SEQ ID NO:105 and a light chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:109.

In some embodiments, the anti-LILRB antibodies include a vhCDR1 comprising SEQ ID NO:106, a vhCDR2 comprising SEQ ID NO:107, a vhCDR3 comprising SEQ ID NO:108, a vlCDR1 comprising SEQ ID NO:110, a vlCDR2 comprising SEQ ID NO:111, and a vlCDR3 comprising SEQ ID NO:112. In some embodiments, one or more of such 6 CDRs have from 1, 2, 3, 4 or 5 amino acid modifications. In further embodiments, a single CDR contains 1 or 2 amino acid substitutions, and the modified anti-LILRB antibodies retain binding to human LILRB.

In some embodiments, the anti-LILRB antibodies in the present disclosure include a heavy chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%9, 97%, 98%, 99%, or 100%) identical to SEQ ID NO:113 and a light chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:117.

In some embodiments, the anti-LILRB antibodies include a vhCDR1 comprising SEQ ID NO:114, a vhCDR2 comprising SEQ ID NO:115, a vhCDR3 comprising SEQ ID NO:116, a vlCDR1 comprising SEQ ID NO: 118, a vlCDR2 comprising SEQ ID NO: 119, and a vlCDR3 comprising SEQ ID NO: 120. In some embodiments, one or more of such 6 CDRs have from 1, 2, 3, 4 or 5 amino acid modifications. In further embodiments, a single CDR contains 1 or 2 amino acid substitutions, and the modified anti-LILRB antibodies retain binding to human LILRB.

In some embodiments, the anti-LILRB antibodies in the present disclosure include a heavy chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%9, 97%, 98%, 99%, or 100%) identical to SEQ ID NO:121 and a light chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:125.

In some embodiments, the anti-LILRB antibodies include a vhCDR1 comprising SEQ ID NO:122, a vhCDR2 comprising SEQ ID NO:123, a vhCDR3 comprising SEQ ID NO:124, a vlCDR1 comprising SEQ ID NO: 126, a vlCDR2 comprising SEQ ID NO:127, and a vlCDR3 comprising SEQ ID NO: 128. In some embodiments, one or more of such 6 CDRs have from 1, 2, 3, 4 or 5 amino acid modifications. In further embodiments, a single CDR contains 1 or 2 amino acid substitutions, and the modified anti-LILRB antibodies retain binding to human LILRB.

In some embodiments, the anti-LILRB antibodies in the present disclosure include a heavy chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%9, 97%, 98%, 99%, or 100%) identical to SEQ ID NO:129 and a light chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:133.

In some embodiments, the anti-LILRB antibodies include a vhCDR1 comprising SEQ ID NO:130, a vhCDR2 comprising SEQ ID NO:131, a vhCDR3 comprising SEQ ID NO:132, a vlCDR1 comprising SEQ ID NO: 134, a vlCDR2 comprising SEQ ID NO:135, and a vlCDR3 comprising SEQ ID NO:136. In some embodiments, one or more of such 6 CDRs have from 1, 2, 3, 4 or 5 amino acid modifications. In further embodiments, a single CDR contains 1 or 2 amino acid substitutions, and the modified anti-LILRB antibodies retain binding to human LILRB.

In some embodiments, the anti-LILRB antibodies in the present disclosure include a heavy chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%9, 97%, 98%, 99%, or 100%) identical to SEQ ID NO:137 and a light chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:141.

In some embodiments, the anti-LILRB antibodies include a vhCDR1 comprising SEQ ID NO:138, a vhCDR2 comprising SEQ ID NO:139, a vhCDR3 comprising SEQ ID NO:140, a vlCDR1 comprising SEQ ID NO: 142, a vlCDR2 comprising SEQ ID NO:143, and a vlCDR3 comprising SEQ ID NO: 144. In some embodiments, one or more of such 6 CDRs have from 1, 2, 3, 4 or 5 amino acid modifications. In further embodiments, a single CDR contains 1 or 2 amino acid substitutions, and the modified anti-LILRB antibodies retain binding to human LILRB.

In some embodiments, the anti-LILRB antibodies in the present disclosure include a heavy chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%9, 97%, 98%, 99%, or 100%) identical to SEQ ID NO:145 and a light chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:149.

In some embodiments, the anti-LILRB antibodies include a vhCDR1 comprising SEQ ID NO: 146, a vhCDR2 comprising SEQ ID NO:147, a vhCDR3 comprising SEQ ID NO:148, a vlCDR1 comprising SEQ ID NO:150, a vlCDR2 comprising SEQ ID NO:151, and a vlCDR3 comprising SEQ ID NO: 152. In some embodiments, one or more of such 6 CDRs have from 1, 2, 3, 4 or 5 amino acid modifications. In further embodiments, a single CDR contains 1 or 2 amino acid substitutions, and the modified anti-LILRB antibodies retain binding to human LILRB.

In some embodiments, the anti-LILRB antibodies in the present disclosure include a heavy chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%9, 97%, 98%, 99%, or 100%) identical to SEQ ID NO:153 and a light chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:157.

In some embodiments, the anti-LILRB antibodies include a vhCDR1 comprising SEQ ID NO:154, a vhCDR2 comprising SEQ ID NO:155, a vhCDR3 comprising SEQ ID NO:156, a vlCDR1 comprising SEQ ID NO:158, a vlCDR2 comprising SEQ ID NO:159, and a vlCDR3 comprising SEQ ID NO:160. In some embodiments, one or more of such 6 CDRs have from 1, 2, 3, 4 or 5 amino acid modifications. In further embodiments, a single CDR contains 1 or 2 amino acid substitutions, and the modified anti-LILRB antibodies retain binding to human LILRB.

In some embodiments, the anti-LILRB antibodies in the present disclosure include a heavy chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%9, 97%, 98%, 99%, or 100%) identical to SEQ ID NO:161 and a light chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:165.

In some embodiments, the anti-LILRB antibodies include a vhCDR1 comprising SEQ ID NO:162, a vhCDR2 comprising SEQ ID NO:163, a vhCDR3 comprising SEQ ID NO:164, a vlCDR1 comprising SEQ ID NO: 166, a vlCDR2 comprising SEQ ID NO:167, and a vlCDR3 comprising SEQ ID NO: 168. In some embodiments, one or more of such 6 CDRs have from 1, 2, 3, 4 or 5 amino acid modifications. In further embodiments, a single CDR contains 1 or 2 amino acid substitutions, and the modified anti-LILRB antibodies retain binding to human LILRB.

In some embodiments, the anti-LILRB antibodies in the present disclosure include a heavy chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%9, 97%, 98%, 99%, or 100%) identical to SEQ ID NO:169 and a light chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:173.

In some embodiments, the anti-LILRB antibodies include a vhCDR1 comprising SEQ ID NO:170, a vhCDR2 comprising SEQ ID NO:171, a vhCDR3 comprising SEQ ID NO:172, a vlCDR1 comprising SEQ ID NO: 174, a vlCDR2 comprising SEQ ID NO:175, and a vlCDR3 comprising SEQ ID NO: 176. In some embodiments, one or more of such 6 CDRs have from 1, 2, 3, 4 or 5 amino acid modifications. In further embodiments, a single CDR contains 1 or 2 amino acid substitutions, and the modified anti-LILRB antibodies retain binding to human LILRB.

In some embodiments, the anti-LILRB antibodies in the present disclosure include a heavy chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%9, 97%, 98%, 99%, or 100%) identical to SEQ ID NO:177 and a light chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:181.

In some embodiments, the anti-LILRB antibodies include a vhCDR1 comprising SEQ ID NO:178, a vhCDR2 comprising SEQ ID NO:179, a vhCDR3 comprising SEQ ID NO:180, a vlCDR1 comprising SEQ ID NO: 182, a vlCDR2 comprising SEQ ID NO:183, and a vlCDR3 comprising SEQ ID NO: 184. In some embodiments, one or more of such 6 CDRs have from 1, 2, 3, 4 or 5 amino acid modifications. In further embodiments, a single CDR contains 1 or 2 amino acid substitutions, and the modified anti-LILRB antibodies retain binding to human LILRB.

In some embodiments, the anti-LILRB antibodies in the present disclosure include a heavy chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%9, 97%, 98%, 99%, or 100%) identical to SEQ ID NO:185 and a light chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:189.

In some embodiments, the anti-LILRB antibodies include a vhCDR1 comprising SEQ ID NO:186, a vhCDR2 comprising SEQ ID NO:187, a vhCDR3 comprising SEQ ID NO:188, a vlCDR1 comprising SEQ ID NO: 190, a vlCDR2 comprising SEQ ID NO:191, and a vlCDR3 comprising SEQ ID NO: 192. In some embodiments, one or more of such 6 CDRs have from 1, 2, 3, 4 or 5 amino acid modifications. In further embodiments, a single CDR contains 1 or 2 amino acid substitutions, and the modified anti-LILRB antibodies retain binding to human LILRB.

In some embodiments, the anti-LILRB antibodies in the present disclosure include a heavy chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%9, 97%, 98%, 99%, or 100%) identical to SEQ ID NO:193 and a light chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:197.

In some embodiments, the anti-LILRB antibodies include a vhCDR1 comprising SEQ ID NO:194, a vhCDR2 comprising SEQ ID NO:195, a vhCDR3 comprising SEQ ID NO:196, a vlCDR1 comprising SEQ ID NO: 198, a vlCDR2 comprising SEQ ID NO:199, and a vlCDR3 comprising SEQ ID NO:200. In some embodiments, one or more of such 6 CDRs have from 1, 2, 3, 4 or 5 amino acid modifications. In further embodiments, a single CDR contains 1 or 2 amino acid substitutions, and the modified anti-LILRB antibodies retain binding to human LILRB.

In some embodiments, the anti-LILRB antibodies in the present disclosure include a heavy chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%9, 97%, 98%, 99%, or 100%) identical to SEQ ID NO:201 and a light chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:204.

In some embodiments, the anti-LILRB antibodies include a vhCDR1 comprising SEQ ID NO:202, a vhCDR2 comprising SEQ ID NO:203, a vlCDR1 comprising SEQ ID NO:205, a vlCDR2 comprising SEQ ID NO:206, and a vlCDR3 comprising SEQ ID NO:207. In some embodiments, one or more of such 6 CDRs have from 1, 2, 3, 4 or 5 amino acid modifications. In further embodiments, a single CDR contains 1 or 2 amino acid substitutions, and the modified anti-LILRB antibodies retain binding to human LILRB.

In some embodiments, the anti-LILRB antibodies in the present disclosure include a heavy chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%9, 97%, 98%, 99%, or 100%) identical to SEQ ID NO:208 and a light chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:212.

In some embodiments, the anti-LILRB antibodies include a vhCDR1 comprising SEQ ID NO:209, a vhCDR2 comprising SEQ ID NO:210, a vhCDR3 comprising SEQ ID NO:211, a vlCDR1 comprising SEQ ID NO:213, a vlCDR2 comprising SEQ ID NO:214, and a vlCDR3 comprising SEQ ID NO:215. In some embodiments, one or more of such 6 CDRs have from 1, 2, 3, 4 or 5 amino acid modifications. In further embodiments, a single CDR contains 1 or 2 amino acid substitutions, and the modified anti-LILRB antibodies retain binding to human LILRB.

In addition to the sequence variants described herein in the heavy chain and light chain variable regions and/or CDRs, changes in the framework region(s) of the heavy and/or light variable region(s) can be made. In some embodiments, variants in the framework regions (e.g., excluding the CDRs) retain at least about 80, 85, 90 or 95% identity to a germline sequence. Variants can be made to retain at least about 80, 85, 90 or 95% identity to any one of the light chain V-GENE, light chain J-GENE, heavy chain V-GENE, heavy chain J-GENE, and heavy chain D-GENE alleles.

In some embodiments, variations are made in the framework regions that retain at least 80, 85, 90 or 95% identity to the germline gene sequences, while keeping 6 CDRs unchanged.

In some embodiments, variations are made in both the framework regions that retain at least 80, 85, 90 or 95% identity to germline gene sequences. The CDRs can have amino acid modifications (e.g., from 1, 2, 3, 4 or 5 amino acid modifications in the set of CDRs (that is, the CDRs can be modified as long as the total number of changes in the set of 6 CDRs is less than 6 amino acid modifications, with any combination of CDRs being changed; e.g., there may be one change in vlCDR1, two in vhCDR2, none in vhCDR3, etc.). In some embodiments, CDR1 and/or CDR2 can have amino acid modifications (e.g., from 1, 2, 3, 4 or 5 amino acid modifications in either CDR1, CDR2, or both), while CDR3 does not contain modifications.

By selecting amino acid sequences of CDRs and/or variable regions of a heavy chain and a light chain from those described herein and combining them with amino acid sequences of framework regions and/or constant regions of a heavy chain and a light chain of an antibody as appropriate, a person skilled in the art will be able to design an anti-LILRB antibody according to the present invention. The antibody framework regions and/or constant region (Fc domain) described in the current invention can derive from an antibody of any species, such as from human, rabbit, dog, cat, mouse, horse or monkey.

In some embodiments, the constant region is derived from human, and includes a heavy chain constant region derived from those of IgG, IgA, IgM, IgE, and IgD subtypes or variants thereof, and a light chain constant region derived from kappa or lambda subtypes or variants thereof. In some embodiments, the heavy chain constant region is derived from a human IgG, including IgG1, IgG2, IgG3, and IgG4. In some embodiments, the amino acid sequence of the heavy chain constant region is at least 80%, 85%, 90%, or 95% identical to a human IgG1, IgG2, IgG3, or IgG4 constant region. In some other embodiments, the amino acid sequence of the constant region is at least 80%, 85%, 90%, or 95% identical to an antibody constant region from another mammal, such as rabbit, dog, cat, mouse, horse or monkey. In some embodiments, the antibody constant region includes a hinge, a CH2 domain, a CH3 domain and optionally a CH1 domain.

In some embodiments, the antibodies described herein can be derived from a mixture from different species, e.g., forming a chimeric antibody and/or a humanized antibody. In general, both “chimeric antibodies” and “humanized antibodies” refer to antibodies that combine regions from more than one species. For example, “chimeric antibodies” traditionally comprise variable region(s) from a mouse (or rat, in some cases) and the constant region(s) from a human. “Humanized antibodies” generally refer to non-human antibodies that have had the variable-domain framework regions swapped for sequences found in human antibodies. Generally, in a humanized antibody, the entire antibody, except the CDRs, is encoded by a polynucleotide of human origin or is identical to such an antibody except within its CDRs. The CDRs, some or all of which are encoded by nucleic acids originating in a non-human organism, are grafted into the beta-sheet framework of a human antibody variable region to create an antibody, the specificity of which is determined by the engrafted CDRs. The creation of such antibodies is described in, e.g., WO 92/11018, Jones, 1986, Nature 321:522-525, Verhoeyen et al., 1988, Science 239:1534-1536, all entirely incorporated by reference. “Backmutation” of selected acceptor framework residues to the corresponding donor residues is often required to regain affinity that is lost in the initial grafted construct (U.S. Pat. Nos. 5,530,101; 5,585,089; 5,693,761; 5,693,762; 6,180,370; 5,859,205; 5,821,337; 6,054,297; 6,407,213, all entirely incorporated by reference). The humanized antibody optimally also will comprise at least a portion of an immunoglobulin constant region, typically that of a human immunoglobulin, and thus will typically comprise a human Fc region. Humanized antibodies can also be generated using mice with a genetically engineered immune system, as described for example in Roque et al., 2004, Biotechnol. Prog. 20:639-654, entirely incorporated by reference. A variety of techniques and methods for humanizing and reshaping non-human antibodies are well known in the art (See Tsurushita & Vasquez, 2004, Humanization of Monoclonal Antibodies, Molecular Biology of B Cells, 533-545, Elsevier Science (USA), and references cited therein, all entirely incorporated by reference). Humanization methods include but are not limited to methods described in Jones et al., 1986, Nature 321:522-525; Riechmann et al., 1988; Nature 332:323-329; Verhoeyen et al., 1988, Science, 239:1534-1536; Queen et al., 1989, Proc Natl Acad Sci, USA 86:10029-33; He et al., 1998, J. Immunol. 160: 1029-1035; Carter et al., 1992, Proc Natl Acad Sci, USA 89:4285-9, Presta et al., 1997, Cancer Res. 57(20):4593-9; Gorman et al., 1991, Proc. Natl. Acad. Sci. USA 88:4181-4185; O'Connor et al., 1998, Protein Eng 11:321-8, all entirely incorporated by reference. Humanization or other methods of reducing the immunogenicity of nonhuman antibody variable regions may include resurfacing methods, as described for example in Roguska et al., 1994, Proc. Natl. Acad. Sci. USA 91:969-973, entirely incorporated by reference. Other humanization methods may involve the grafting of only parts of the CDRs, including but not limited to methods described in Tan et al., 2002, J. Immunol. 169:1119-1125; De Pascalis et al., 2002, J. Immunol. 169:3076-3084, all entirely incorporated by reference.

In some embodiments, the antibodies of the current invention comprise a heavy chain variable region derived from a particular human germline heavy chain immunoglobulin gene and/or a light chain variable region derived from a particular human germline light chain immunoglobulin gene. Such antibodies may contain amino acid differences as compared to the human germline sequences, due to, for example, naturally-occurring somatic mutations or intentional introduction of site-directed mutation. However, a humanized antibody typically is at least 80% identical in amino acids sequence to an amino acid sequence encoded by a human germline immunoglobulin gene and contains amino acid residues that identify the antibody as being derived from human sequences when compared to the germline immunoglobulin amino acid sequences of other species (e.g., murine germline sequences). In certain cases, a humanized antibody may be at least 95, 96, 97, 98 or 99%, or even at least 96%, 97%, 98%, or 99% identical in amino acid sequence to the amino acid sequence encoded by the human germline immunoglobulin gene. Typically, a humanized antibody derived from a particular human germline sequence will display no more than 10-20 amino acid differences from the amino acid sequence encoded by the human germline immunoglobulin gene. In certain cases, the humanized antibody may display no more than 5, or even no more than 4, 3, 2, or 1 amino acid difference from the amino acid sequence encoded by the germline immunoglobulin gene.

In some embodiments, the antibodies of the current disclosure are humanized and affinity matured, as is known in the art. Structure-based methods may be employed for humanization and affinity maturation, for example as described in U.S. Pat. No. 7,657,380. Selection based methods may be employed to humanize and/or affinity mature antibody variable regions, including but not limited to methods described in Wu et al., 1999, J. Mol. Biol. 294:151-162; Baca et al., 1997, J. Biol. Chem. 272(16):10678-10684; Rosok et al., 1996, J. Biol. Chem. 271(37): 22611-22618; Rader et al., 1998, Proc. Natl. Acad. Sci. USA 95: 8910-8915; Krauss et al., 2003, Protein Engineering 16(10):753-759, all entirely incorporated by reference.

II. Characteristics of the Antibodies

In some embodiments, the anti-LILRB1 and anti-LILRB2 antibodies described herein bind to human LILRB1 or LILRB2 respectively. In some embodiments, binding of the anti-LILBR antibodies to human LILRB is measured by flow cytometry.

In some embodiments, the anti-LILRB antibodies described herein display low immunogenicity when administered into human subjects. These antibodies can contain an Fc domain derived from human IgG1, human IgG2 or human IgG3. In some embodiments, these antibodies are humanized using the framework regions derived from human immunoglobulins.

In some embodiments, the anti-LILRB antibodies affect the responsiveness of T cells. In some embodiments the anti-LILRB antibodies regulate surface expression of activation markers in response to different types of T cell stimulation. In some embodiments the anti-LILRB antibodies regulate cytokine production by PBMCs in response to T cell stimulation.

In some embodiments, anti-LILRB1 and LILRB2 antibodies described act as LILRB antagonists. As a result, such anti-LILRB1 and LILRB2 antibodies inhibit the activity of LILRB1 and LILRB2, respectively.

In some other embodiments, anti-LIRB1 and anti-LILRB2 antibodies described herein act as agonists. As a result, such anti-LILRB1 and anti-LILRB2 antibodies promote the activity of LILRB1 and LILRB2, respectively.

Effects of the anti-LILRB antibodies on T cell function can be assayed using a variety of methods known in the art and described herein. Accordingly, the anti-LILRB antibodies can serve as LILRB antagonists or LILRB agonists.

III. Nucleic Acids of the Invention

Nucleic acids encoding the anti-LILRB antibodies described herein are also encompassed by the present disclosure, as well as expression vectors containing such nucleic acids and host cells transformed with such nucleic acids and/or expression vectors. As will be appreciated by those in the art, the protein sequences depicted herein can be encoded by any number of possible nucleic acid sequences due to the degeneracy of the genetic code, and one of skill in the art could readily identify such nucleic acid sequences based on the amino acid sequences provided herein.

In some embodiments, nucleic acid compositions encoding the anti-LILRB antibodies and/or LILRB-binding domains are also encompassed by the invention. As will be appreciated by those in the art, in the case of antigen binding domains, the nucleic acid compositions generally include a first nucleic acid encoding the heavy chain variable region and a second nucleic acid encoding the light chain variable region. In the case of scFvs, a single nucleic acid encoding the heavy chain variable region and light chain variable region, separated by a linker described herein, can be made. In the case of traditional antibodies, the nucleic acid compositions generally include a first nucleic acid encoding the heavy chain and a second nucleic acid encoding the light chain, which will, upon expression in a cell, spontaneously assemble into the “traditional” tetrameric format of two heavy chains and two light chains.

In some embodiments, the nucleic acid compositions encoding the anti-LILRB antibodies and/or LILRB-binding domains are codon optimized versions or variants.

As is known in the art, the nucleic acids encoding the components of the invention can be incorporated into expression vectors, and depending on the host cells, used to produce the antibodies of the invention. These two nucleic acids can be incorporated into a single expression vector or into two different expression vectors. Generally, the nucleic acids can be operably linked to any number of regulatory elements (promoters, origin of replication, selectable markers, ribosomal binding sites, inducers, etc.) in an expression vector. The expression vectors can be extra-chromosomal or integrating vectors.

The nucleic acids and/or expression vectors of the current invention can be introduced into any type of host cells, which are well known in the art, including mammalian, bacterial, yeast, insect and fungal cells. After transfection, single cell clones can be isolated for cell bank generation using methods known in the art, such as limited dilution, ELISA, FACS, microscopy, or Clonepix. Clones can be cultured under conditions suitable for bio-reactor scale-up and maintained expression of the antibodies. The antibodies can be isolated and purified using methods known in the art including centrifugation, depth filtration, cell lysis, homogenization, freeze-thawing, affinity purification, gel filtration, ion exchange chromatography, hydrophobic interaction exchange chromatography, and mixed-mode chromatography.

IV. Therapeutic Applications

The current disclosure provides a method of modulating an immune response in a subject, and the method includes administering to the subject an effective amount of an anti-LILRB antibody described herein, or a pharmaceutical composition containing an anti-LILRB antibody.

In some embodiments, the methods of modulating an immune response encompassed by the present disclosure comprises inhibiting LILRB activity in a subject, and in further embodiments, such methods comprise administering to the subject an effective amount of an anti-LILRB antibody that acts as a LILRB antagonist, or by administering a pharmaceutical composition containing an antagonistic anti-LILRB antibody.

In some embodiments, the methods of modulating an immune response encompassed by the present disclosure comprises promoting LILRB activity in a subject, and in further embodiments, such methods comprise administering to the subject an effective amount of an anti-LILRB antibody that acts as a LILRB agonist, or by administering a pharmaceutical composition containing an agonistic anti-LILRB antibody.

In some embodiments, an antagonist may stimulate and immune response. In other embodiments an antagonistic may inhibit an immune response. In some embodiments an agonist may stimulate an immune response. In other embodiments an agonist may inhibit an immune response.

The present disclosure also provides methods of treating cancer in a subject, and such methods include administering to the subject an effective amount of an anti-LILRB antibody described herein, or a pharmaceutical composition containing such anti-LILRB antibody. In some embodiments, the cancer to be treated expresses LILRB on the cancer cell surface. In some embodiments, the cancer to be treated upregulates LILRB compared to the corresponding non-cancerous tissue. In some embodiments, the subject to be treated expresses LILRB on one or more types of immune cells including lymphoid cells, myeloid cells, monocytes, monocyte-derived osteoclasts, granulocytes, dendritic cells, osteoclasts, and progenitor mast cells. In some embodiments, the subject to be treated expresses a high level of LILRB on one or more types of immune cells including monocytes, monocyte-derived osteoclasts, granulocytes, dendritic cells, osteoclasts, and progenitor mast cells.

In some embodiments, the cancer is myeloid leukemia, B lymphoid leukemia, or myeloma.

In some other embodiments, the cancer is brain cancer, bladder cancer, breast cancer, cervical cancer, endometrial cancer, esophageal cancer, leukemia, lung cancer, liver cancer, melanoma, ovarian cancer, pancreatic cancer, prostate cancer, rectal cancer, renal cancer, testicular cancer, or uterine cancer. In yet other embodiments, the cancer is a vascularized tumor, squamous cell carcinoma, adenocarcinoma, small cell carcinoma, neuroblastoma, sarcoma (e.g., an angiosarcoma or chondrosarcoma), larynx cancer, parotid cancer, biliary tract cancer, thyroid cancer, acral lentiginous melanoma, actinic keratoses, acute lymphocytic leukemia, acute myeloid leukemia, adenoid cystic carcinoma, adenomas, adenosarcoma, adenosquamous carcinoma, anal canal cancer, anal cancer, anorectum cancer, astrocytic tumor, bartholin gland carcinoma, basal cell carcinoma, biliary cancer, bone cancer, bone marrow cancer, bronchial cancer, bronchial gland carcinoma, carcinoid, cholangiocarcinoma, chondosarcoma, choroid plexus papilloma/carcinoma, chronic lymphocytic leukemia, chronic myeloid leukemia, clear cell carcinoma, connective tissue cancer, cystadenoma, digestive system cancer, duodenum cancer, endocrine system cancer, endodermal sinus tumor, endometrial hyperplasia, endometrial stromal sarcoma, endometrioid adenocarcinoma, endothelial cell cancer, ependymal cancer, epithelial cell cancer, Ewing's sarcoma, eye and orbit cancer, female genital cancer, focal nodular hyperplasia, gallbladder cancer, gastric antrum cancer, gastric fundus cancer, gastrinoma, glioblastoma, glucagonoma, heart cancer, hemangiblastomas, hemangioendothelioma, hemangiomas, hepatic adenoma, hepatic adenomatosis, hepatobiliary cancer, hepatocellular carcinoma, Hodgkin's disease, ileum cancer, insulinoma, intraepithelial neoplasia, interepithelial squamous cell neoplasia, intrahepatic bile duct cancer, invasive squamous cell carcinoma, jejunum cancer, joint cancer, Kaposi's sarcoma, pelvic cancer, large cell carcinoma, large intestine cancer, leiomyosarcoma, lentigo maligna melanomas, lymphoma, male genital cancer, malignant melanoma, malignant mesothelial tumors, medulloblastoma, medulloepithelioma, meningeal cancer, mesothelial cancer, metastatic carcinoma, mouth cancer, mucoepidermoid carcinoma, multiple myeloma, muscle cancer, nasal tract cancer, nervous system cancer, neuroepithelial adenocarcinoma nodular melanoma, non-epithelial skin cancer, oat cell carcinoma, oligodendroglial cancer, oral cavity cancer, osteosarcoma, papillary serous adenocarcinoma, penile cancer, pharynx cancer, pituitary tumors, plasmacytoma, pseudosarcoma, pulmonary blastoma, rectal cancer, renal cell carcinoma, respiratory system cancer, retinoblastoma, rhabdomyosarcoma, sarcoma, serous carcinoma, sinus cancer, skin cancer, small cell carcinoma, small intestine cancer, smooth muscle cancer, soft tissue cancer, somatostatin-secreting tumor, spine cancer, squamous cell carcinoma, striated muscle cancer, submesothelial cancer, superficial spreading melanoma, T cell leukemia, tongue cancer, undifferentiated carcinoma, ureter cancer, urethra cancer, urinary bladder cancer, urinary system cancer, uterine cervix cancer, uterine corpus cancer, uveal melanoma, vaginal cancer, verrucous carcinoma, VIPoma, vulva cancer, well-differentiated carcinoma, or Wilms tumor.

In some other embodiments, the cancer to be treated is a non-Hodgkin's lymphoma, such as a B-cell lymphoma or a T-cell lymphoma. In certain embodiments, the non-Hodgkin's lymphoma is a B-cell lymphoma, such as a diffuse large B-cell lymphoma, primary mediastinal B-cell lymphoma, follicular lymphoma, small lymphocytic lymphoma, mantle cell lymphoma, marginal zone B-cell lymphoma, extranodal marginal zone B-cell lymphoma, nodal marginal zone B-cell lymphoma, splenic marginal zone B-cell lymphoma, Burkitt lymphoma, lymphoplasmacytic lymphoma, hairy cell leukemia, or primary central nervous system (CNS) lymphoma. In certain other embodiments, the non-Hodgkin's lymphoma is a T-cell lymphoma, such as a precursor T-lymphoblastic lymphoma, peripheral T-cell lymphoma, cutaneous T-cell lymphoma, angioimmunoblastic T-cell lymphoma, extranodal natural killer/T-cell lymphoma, enteropathy type T-cell lymphoma, subcutaneous panniculitis-like T-cell lymphoma, anaplastic large cell lymphoma, or peripheral T-cell lymphoma.

The present disclosure also provides methods of treating autoimmune or inflammatory disorders in a subject, and the method includes administering to the subject an effective amount of an anti-LILRB antibody that acts as a modulator of LILRB. In some embodiments, the subject to be treated expresses LILRB on one or more types of immune cells including lymphoid cells, myeloid cells, monocytes, monocyte-derived osteoclasts, granulocytes, dendritic cells, osteoclasts, and progenitor mast cells. In some embodiments, the subject to be treated expresses a high level of LILRB on one or more types of immune cells including lymphoid cells, myeloid cells, monocytes, monocyte-derived osteoclasts, granulocytes, dendritic cells, osteoclasts, and progenitor mast cells. In some embodiments, LILRB is expressed in the subject at a high level on autoreactive immune cells (e.g., T cells, B cells, natural killer cells, dendritic cells, endothelial cells, and macrophages at sites where the autoimmune disease develops, for example, lymph nodes and central nervous system in the subject suffering from multiple sclerosis, joints in the subject suffering from Rheumatoid arthritis, and gastrointestinal tract in the subject suffering from Celiac disease). Administering an anti-LILRB antibody that acts as a LILRB antagonist can inhibit LILRB activity. Administering an anti-LILRB antibody that acts as a LILRB agonist can promote LILRB activity.

In some embodiments, the autoimmune or inflammatory disorder to treated is asthma, multiple sclerosis, Addison's disease, amyotrophic lateral sclerosis, Crohn's disease, Cushing's Syndrome, diabetes mellitus type 1, graft versus host disease, Graves' disease, Guillain-Barre syndrome, lupus erythematosus, psoriasis, psoriatic arthritis, rheumatoid arthritis, sarcoidosis, scleroderma, systemic lupus erythematosus, transplant rejection, or vasculitis.

In some other embodiments, the autoimmune disorders to be treated include, but are not limited to, Acute disseminated encephalomyelitis (ADEM), Agammaglobulinemia, Alopecia areata, Ankylosing Spondylitis, Antiphospholipid syndrome, Antisynthetase syndrome, Atopic allergy, Atopic dermatitis, Autoimmune aplastic anemia, Autoimmune cardiomyopathy, Autoimmune enteropathy, Autoimmune hemolytic anemia, Autoimmune hepatitis, Autoimmune inner ear disease, Autoimmune lymphoproliferative syndrome, Autoimmune pancreatitis, Autoimmune peripheral neuropathy, Autoimmune polyendocrine syndrome, Autoimmune progesterone dermatitis, Autoimmune thrombocytopenic purpura, Autoimmune urticaria, Autoimmune uveitis, Balo disease/Balo concentric sclerosis, Behcet's disease, Berger's disease, Bickerstaffs encephalitis, Blau syndrome, Bullous pemphigoid, Cancer, Castleman's disease, Celiac disease, Chagas disease, Chronic inflammatory demyelinating polyneuropathy, Chronic inflammatory demyelinating polyneuropathy, Chronic obstructive pulmonary disease, Chronic recurrent multifocal osteomyelitis, Churg-Strauss syndrome, Cicatricial pemphigoid, Cogan syndrome, Cold agglutinin disease, Complement component 2 deficiency, Contact dermatitis, Cranial arteritis, CREST syndrome, Cutaneous leukocytoclastic angiitis, Dego's disease, Dercum's disease, Dermatitis herpetiformis, Dermatomyositis, Diffuse cutaneous systemic sclerosis, Discoid lupus erythematosus, Dressler's syndrome, Drug-induced lupus, Eczema, Endometriosis, Eosinophilic fasciitis, Eosinophilic gastroenteritis, Eosinophilic pneumonia, Epidermolysis bullosa acquisita, Erythema nodosum, Erythroblastosis fetalis, Essential mixed cryoglobulinemia, Evan's syndrome, Fibrodysplasia ossificans progressiva, Fibrosing alveolitis (or Idiopathic pulmonary fibrosis), Gastritis, Gastrointestinal pemphigoid, Glomerulonephritis, Goodpasture's syndrome, Hashimoto's encephalopathy, Hashimoto's thyroiditis, Henoch-Schonlein purpura, Herpes gestationis aka Gestational Pemphigoid, Hidradenitis suppurativa, Hughes-Stovin syndrome, Hypogammaglobulinemi, Idiopathic inflammatory demyelinating diseases, Idiopathic pulmonary fibrosis, Idiopathic thrombocytopenic purpura, IgA nephropathy, Inclusion body myositis, Interstitial cystitis, Juvenile idiopathic arthritis aka Juvenile rheumatoid arthritis, Kawasaki's disease, Lambert-Eaton myasthenic syndrome, Leukocytoclastic vasculitis, Lichen planus, Lichen sclerosus, Linear IgA disease, Lupoid hepatitis aka Autoimmune hepatitis, Majeed syndrome, Microscopic colitis, Microscopic polyangiitis, Miller-Fisher syndrome, Mixed connective tissue disease, Morphea, Mucha-Habermann disease aka Pityriasis lichenoides et varioliformis acuta, Multiple sclerosis, Myasthenia gravis, Myositis, Meniere's disease, Narcolepsy, Neuromyelitis optica, Neuromyotonia, Occular cicatricial pemphigoid, Opsoclonus myoclonus syndrome, Ord's thyroiditis, Palindromic rheumatism, PANDAS (pediatric autoimmune neuropsychiatric disorders associated with streptococcus), Paraneoplastic cerebellar degeneration, Paroxysmal nocturnal hemoglobinuria (PNH), Parry Romberg syndrome, Pars planitis, Parsonage-Turner syndrome, Pemphigus vulgaris, Perivenous encephalomyelitis, Pernicious anaemia, POEMS syndrome, Polyarteritis nodosa, Polymyalgia rheumatica, Polymyositis, Primary biliary cirrhosis, Primary sclerosing cholangitis, Progressive inflammatory neuropathy, Pure red cell aplasia, Pyoderma gangrenosum, Rasmussen's encephalitis, Raynaud phenomenon, Reiter's syndrome, Relapsing polychondritis, Restless leg syndrome, Retroperitoneal fibrosis, Rheumatic fever, Schizophrenia, Schmidt syndrome, Schnitzler syndrome, Scleritis, Serum Sickness, Sjögren's syndrome, Spondyloarthropathy, Stiff person syndrome, Still's disease, Subacute bacterial endocarditis (SBE), Susac's syndrome, Sweet's syndrome, Sydenham chorea, Sympathetic ophthalmia, Takayasu's arteritis, Temporal arteritis, Thrombocytopenia, Tolosa-Hunt syndrome, Transverse myelitis, Ulcerative colitis, Undifferentiated spondyloarthropathy, Urticarial vasculitis, Vitiligo, Wegener's granulomatosis.

The present disclosure also provides methods of treating allergic inflammation in a subject, and the method includes administering to the subject an effective amount of any one of the anti-LILRB antibodies described herein, or any one of the compositions described herein.

In some embodiments, the allergic inflammation to be treated may be related to allergic asthma, atopic dermatitis, allergic rhinitis, allergic conjunctivitis.

The present disclosure also provides methods of modulating osteoclast differentiation, and the method includes administering to the subject an effective amount of any one of the anti-LILRB antibodies described herein, or any one of the compositions described herein.

In some embodiments, modulating osteoclast differentiation may be particularly useful to treat bone loss or bone resorption in patients suffering or susceptible of suffering from a condition selected from the group consisting of osteoporosis, osteodystrophy, osteopenia, osteomalacia, hyperparathyroidism, hyperthyroidism, hypogonadism, thyrotoxicosis, systemic mastocytosis, adult hypophosphatasia, hyperadrenocorticism, osteogenesis imperfecta, Paget's disease, Cushing's disease/syndrome, Tumer syndrome, Gaucher disease, Ehlers-Danlos syndrome, Marfan's syndrome, Menkes' syndrome, Fanconi's syndrome, multiple myeloma, hypercalcemia, hypocalcemia, arthritides, periodontal disease, rickets (including vitamin D dependent, type I and II, and x-linked hypophosphatemic rickets) or other form of vitamin D deficiency such as vitamin D deficiency associated with chronic kidney disease or kidney failure, fibrogenesis imperfecta ossium, osteosclerotic disorders such as pycnodysostosis and damage caused by macrophage-mediated inflammatory processes.

V. Combination Therapy

Anti-LILRB antibodies described herein can be used in combination with additional therapeutic agents to treat cancer, autoimmune disorders, and allergic inflammation. Anti-LILRB antibodies can also be used in combination with additional therapeutic agents to modulate osteoclast differentiation

Exemplary therapeutic agents that may be used as part of a combination therapy in treating cancer, include, for example, radiation, mitomycin, tretinoin, ribomustin, gemcitabine, vincristine, etoposide, cladribine, mitobronitol, methotrexate, doxorubicin, carboquone, pentostatin, nitracrine, zinostatin, cetrorelix, letrozole, raltitrexed, daunorubicin, fadrozole, fotemustine, thymalfasin, sobuzoxane, nedaplatin, cytarabine, bicalutamide, vinorelbine, vesnarinone, aminoglutethimide, amsacrine, proglumide, elliptinium acetate, ketanserin, doxifluridine, etretinate, isotretinoin, streptozocin, nimustine, vindesine, flutamide, drogenil, butocin, carmofur, razoxane, sizofilan, carboplatin, mitolactol, tegafur, ifosfamide, prednimustine, picibanil, levamisole, teniposide, improsulfan, enocitabine, lisuride, oxymetholone, tamoxifen, progesterone, mepitiostane, epitiostanol, formestane, interferon-alpha, interferon-2 alpha, interferon-beta, interferon-gamma, colony stimulating factor-1, colony stimulating factor-2, denileukin diftitox, interleukin-2, luteinizing hormone releasing factor and variations of the aforementioned agents that may exhibit differential binding to its cognate receptor, and increased or decreased serum half-life.

An additional class of agents that may be used as part of a combination therapy in treating cancer is immune checkpoint inhibitors. Exemplary immune checkpoint inhibitors include agents that inhibit one or more of (i) cytotoxic T-lymphocyte-associated antigen 4 (CTLA4), (ii) programmed cell death protein 1 (PD1), (iii) PDL1, (iv) LAG3, (v) B7-H3, (vi) B7-H4, and (vii) TIM3, such as Ipilimumab, Nivolumab, Pembrolizumab, Avelumab, Durvalumab, and Atezolizumab.

Yet other agents that may be used as part of a combination therapy in treating cancer are monoclonal antibody agents that target non-checkpoint targets (e.g., herceptin) and non-cytotoxic agents (e.g., tyrosine-kinase inhibitors).

Yet other categories of anti-cancer agents include, for example: (i) an inhibitor selected from an ALK Inhibitor, an ATR Inhibitor, an A2A Antagonist, a Base Excision Repair Inhibitor, a Bcr-Abl Tyrosine Kinase Inhibitor, a Bruton's Tyrosine Kinase Inhibitor, a CDC7 Inhibitor, a CHK1 Inhibitor, a Cyclin-Dependent Kinase Inhibitor, a DNA-PK Inhibitor, an Inhibitor of both DNA-PK and mTOR, a DNMT1 Inhibitor, a DNMT1 Inhibitor plus 2-chloro-deoxyadenosine, an HDAC Inhibitor, a Hedgehog Signaling Pathway Inhibitor, an IDO Inhibitor, a JAK Inhibitor, a mTOR Inhibitor, a MEK Inhibitor, a MELK Inhibitor, a MTH1 Inhibitor, a PARP Inhibitor, a Phosphoinositide 3-Kinase Inhibitor, an Inhibitor of both PARP1 and DHODH, a Proteasome Inhibitor, a Topoisomerase-II Inhibitor, a Tyrosine Kinase Inhibitor, a VEGFR Inhibitor, and a WEE1 Inhibitor; (ii) an agonist of OX40, CD137, CD40, GITR, CD27, HVEM, TNFRSF25, or ICOS; and (iii) a cytokine selected from IL-12, IL-15, GM-CSF, and G-CSF. Antibodies of the invention can also be used as an adjunct to surgical removal of cancer from the primary lesion.

Exemplary therapeutic agents that may be used as a part of a combination therapy with the anti-LILRB antibodies for treating, delaying the progression of, preventing a relapse of, or alleviating a symptom of an autoimmune or inflammatory disorder, include, for example, any of a variety of known anti-inflammatory and/or immunosuppressive therapy. In some embodiments, the anti-inflammatory and/or immunosuppressive therapies include, but are not limited to methotrexate, cyclosporin A (including, for example, cyclosporin microemulsion), tacrolimus, corticosteroids, statins, interferon beta, non-steroidal anti-inflammatory agents, and 6-MP (Mercaptopurine, also called 6-Mercaptopurine, or Purinethol).

In some embodiments, the anti-inflammatory and/or immunosuppressive therapies for combining with the anti-LILRB antibodies include, but are not limited to a TOPK inhibitor (e.g., OTS964 ((R)-9-(4-(1-(dimethylamino)propan-2-yl)phenyl)-8-hydroxy-6-methylthieno[2,3-c]quinolin-4(5H)-one) (Oncotherapy Science)), a tyrosine kinase inhibitor (e.g., axitinib, dasatinib, icotinib), a topoisomerase inhibitor (e.g., topotecan), a sphingosine-1-phosphate receptor agonist (e.g., fingolimod, KRP-203), anti-T cell immunoglobulin (e.g. AtGam), anti-IL-2 receptor antibody (e.g. daclizumab), amides (CTX), ifosfamide (IFO), adriamycin (ADM), daunorubicin (DNR), vincristine (VCR), vinblastine (VBL), etoposide (VP16), vermeer (Vumon), carboplatin (CBP), tacrolimus, sirolimus, everolimus, azathioprine, brequinar, leflunomide, LEA-29Y, anti-CD3 antibody (e.g. OKT3), aspirin, B7-CD28 blocking molecules (e.g. belatacept, abatacept), CD40-CD154 blocking molecules (anti-CD40 antibodies), acetaminophen, ibuprofen, naproxen, piroxicam, and anti-inflammatory steroids (e.g. prednisolone or dexamethasone).

In some embodiments, the anti-inflammatory and/or immunosuppressive therapies for combining with the anti-LILRB antibodies include ablation of autoimmune cells, for example, by administration of TNF-alpha, CFA, interleukin-1 (IL-1), proteasome inhibitors, NFκB inhibitors, anti-inflammatory drugs, tissue plasminogen activator (TPA), lipopolysaccharide, UV light, and an intracellular mediator of the TNF-alpha signaling pathway. Such agents induce the apoptosis of autoreactive lymphocytes by interrupting the pathway downstream from TNF-alpha receptor signaling or act downstream of TNF-alpha receptor binding. (Baldwin et al., Ann. Rev. Immunol. (1996) 12:141; Baltimore, Cell (1996) 87:13).

In some embodiments, the anti-LILRB antibodies are used in conjunction with a surgical method of treating or otherwise alleviating autoimmune diseases.

Exemplary therapeutic agents that may be used as a part of a combination therapy with the anti-LILRB antibodies for treating, delaying the progression of, preventing a relapse of, or alleviating a symptom of allergic inflammation, include, for example, any of a variety of known anti-inflammatory and/or immunosuppressive therapies. In some embodiments, the anti-inflammatory and/or immunosuppressive therapies for combining with anti-LILRB antibodies include but are not limited to: short-acting β2-agonists, long-acting β2-agonists, anticholinergics, corticosteroids, systemic corticosteroids, mast cell stabilizers, leukotriene modifiers, methylxanthines, β2-agonists, albuterol, levalbuterol, pirbuterol, artformoterol, formoterol, salmeterol, anticholinergics including ipratropium and tiotropium; corticosteroids including beclomethasone, budesonide, flunisolide, fluticasone, mometasone, triamcinolone, methyprednisolone, prednisolone, prednisone; leukotriene modifiers including montelukast, zafirlukast, and zileuton; mast cell stabilizers including cromolyn and nedocromil; methylxanthines including theophylline; combination drugs including ipratropium and albuterol, fluticasone and salmeterol, budesonide and formoterol; antihistamines including hydroxyzine, diphenhydramine, loratadine, cetirizine, and hydrocortisone; immune system modulating drugs including tacrolimus and pimecrolimus; cyclosporine; azathioprine; mycophenolatemofetil; and combinations thereof.

In other embodiments, therapeutic agents that may be used as a part of a combination therapy with the anti-LILRB antibodies for treating, delaying the progression of, preventing a relapse of, or alleviating a symptom of allergic inflammation, may also include those therapeutic agents specified for autoimmune or inflammatory disorders.

Exemplary therapeutic agents that may be used as a part of a combination therapy with the anti-LILRB antibodies for modulating osteoclast activity include but are not limited to bisphosphonates, calcitonin, estrogen replacement, sclerostin antibodies, RANKL antibodies, parathyroid peptides, strontiumranelate, TNFα inhibitors, colony-stimulating factor-1 inhibitors, colony-stimulating factor-1 receptor inhibitors, cathepsin K inhibitors, V-ATPase inhibitors, and Glucagon-like peptide 2.

The amount of the antibodies and additional therapeutic agents and the relative timing of administration may be selected in order to achieve a desired combined therapeutic effect. For example, when administering a combination therapy to a patient in need of such administration, the therapeutic agents in the combination, or a pharmaceutical composition or compositions comprising the therapeutic agents, may be administered in any order such as, for example, sequentially, concurrently, together, simultaneously and the like. Further, for example, a multi-specific binding protein may be administered during a time when the additional therapeutic agent(s) exerts its prophylactic or therapeutic effect, or vice versa.

VI. Pharmaceutical Composition and Administration

The present disclosure also features pharmaceutical compositions/formulations that contain a therapeutically effective amount of an anti-LILRB antibody described herein. The composition can be formulated for use in a variety of drug delivery systems. One or more physiologically acceptable excipients or carriers can also be included in the composition for proper formulation. Suitable formulations for use in the present disclosure are found in Remington's Pharmaceutical Sciences, Mack Publishing Company, Philadelphia, Pa., 17th ed., 1985. For a brief review of methods for drug delivery, see, e.g., Langer (Science 249:1527-1533, 1990).

The antibodies of the present disclosure can exist in a lyophilized formulation or liquid aqueous pharmaceutical formulation. The aqueous carrier of interest herein is one which is pharmaceutically acceptable (safe and non-toxic for administration to a human) and is useful for the preparation of a liquid formulation. Illustrative carriers include sterile water for injection (SWFI), bacteriostatic water for injection (BWFI), a pH buffered solution (e.g., phosphate-buffered saline), sterile saline solution, Ringer's solution or dextrose solution.

The antibodies of the present disclosure could exist in a lyophilized formulation including the proteins and a lyoprotectant. The lyoprotectant may be sugar, e.g., disaccharides. In certain embodiments, the lyoprotectant is sucrose or maltose. The lyophilized formulation may also include one or more of a buffering agent, a surfactant, a bulking agent, and/or a preservative.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient. It may be administered in the range of 0.1 mg to 1 g and preferably in the range of 0.5 mg to 500 mg of active antibody per administration for adults. Alternatively, a patient's dose can be tailored to the approximate body weight or surface area of the patient. Other factors in determining the appropriate dosage can include the disease or condition to be treated or prevented, the severity of the disease, the route of administration, and the age, sex and medical condition of the patient. Further refinement of the calculations necessary to determine the appropriate dosage for treatment is routinely made by those skilled in the art, especially in light of the dosage information and assays disclosed herein. The dosage can also be determined through the use of known assays for determining dosages used in conjunction with appropriate dose-response data. An individual patient's dosage can be adjusted as the progress of the disease is monitored. Blood levels of the targetable construct or complex in a patient can be measured to see if the dosage needs to be adjusted to reach or maintain an effective concentration. Pharmacogenomics may be used to determine which targetable constructs and/or complexes, and dosages thereof, are most likely to be effective for a given individual (Schmitz et al., Clinica Chimica Acta 308: 43-53, 2001; Steimer et al., Clinica Chimica Acta 308: 33-41, 2001).

Doses may be given once or more times daily, weekly, monthly or yearly, or even once every 2 to 20 years. Persons of ordinary skill in the art can easily estimate repetition rates for dosing based on measured residence times and concentrations of the targetable construct or complex in bodily fluids or tissues. Administration of the present invention could be intravenous, intraarterial, intraperitoneal, intramuscular, subcutaneous, intrapleural, intrathecal, intracavitary, by perfusion through a catheter or by direct intralesional injection. This may be administered once or more times daily, once or more times weekly, once or more times monthly, and once or more times annually.

### EXAMPLES

The invention now being generally described, will be more readily understood by reference to the following examples, which are included merely for purposes of illustration of certain aspects and embodiments of the present invention, and is not intended to limit the invention.

### Example 1—LILRB1 and LILRB2 Monoclonal Antibody Bioacore Affinity Data

Dissociation constants (Kd) for mouse anti-human LILRB1 and LIRB2 antibodies were determined using a Biacore X100 surface plasmon resonance instrument (Cytivia). Recombinant human Fc-tagged extracellular domains of LILRB1 or LILRB2 (LILRB1-Fc or LILRB2-Fc; R&D Systems) were immobilized on a Biacore CM5 biosensor chip by amine coupling at a density of 40-50 relative units (RU). Purified mouse monoclonal antibodies were injected over the biosensor in a concentration series of 3.7, 11, 33, 100, and 300 nM in HBS-EP buffer (10 mM HEPS pH 7.4, 150 M NaCl, 3 mM EDTA, 0.005% v/v Surfactant P20) at a flow rate of 50 μL/min. Regeneration of the chip was achieved by a 30 s injection of 10 mM glycine-HCl, pH 1.7. Affinity parameters were determined by fitting response data using the accompanying Biacore X100 analysis software.

| LILRB1 antibody | K_{d} (M⁻¹) | −log(K_{d}) |
|---|---|---|
| 1B10 | 7.38E−09 | 8.131885 |
| 3E12 | 2.46E−12 | 11.60845 |
| 4C2 | 2.21E−13 | 12.65472 |
| 4A10 | 1.3E−08 | 7.886391 |
| 4A11 | 1.74E−08 | 7.759754 |
| 4D5 | 1.28E−08 | 7.894164 |
| 4H5 | 2.29E−13 | 12.63941 |
| 5B3 | 1.74E−09 | 8.760485 |
| 5E11 | >10E−03 | <3 |
| 5E6 | 6.29E−09 | 8.20128 |
| 5H12 | 5.46E−09 | 8.262807 |
| 6A11 | 4.01E−13 | 12.39736 |
| 6A6 | 1.04E−08 | 7.982132 |
| 6B7 | 2.63E−07 | 6.579381 |

| LILRB2 antibody | K_{d} (M⁻¹) | −log(K_{d}) |
|---|---|---|
| 1A8 | 1.62E−08 | 7.791827 |
| 1C8 | 2.29E−08 | 7.640178 |
| 1G3 | 8.92E−09 | 8.049405 |
| 2F10 | 3.96E−09 | 8.40196 |
| 2G9 | 4.63E−09 | 8.334001 |
| 3B3 | 9.66E−09 | 8.014843 |
| 6B3 | 9.61E−10 | 9.017277 |
| 8D2 | 3.69E−09 | 8.432503 |
| 10A8 | 1.68E−09 | 8.773484 |
| 10D4 | 1.52E−08 | 7.819014 |
| 10F5 | 1.12E−08 | 7.950394 |

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

What is claimed is:

1. An antibody that binds human LILRB1 or LILRB2, the antibody comprising:
a) a heavy chain variable region comprising a vhCDR1, a vhCDR2 and a vhCDR3 from a heavy chain variable region comprising an amino acid sequence of SEQ ID NO:1 and a light chain variable region comprising a vlCDR1, a vlCDR2, and a vlCDR3 from a light chain variable region comprising an amino acid sequence of SEQ ID NO:5;
b) a heavy chain variable region comprising a vhCDR1, a vhCDR2 and a vhCDR3 from a heavy chain variable region comprising an amino acid sequence of SEQ ID NO:9 and a light chain variable region comprising a vlCDR1, a vlCDR2, and a vlCDR3 from a light chain variable region comprising an amino acid sequence of SEQ ID NO:13;
c) a heavy chain variable region comprising a vhCDR1, a vhCDR2 and a vhCDR3 from a heavy chain variable region comprising an amino acid sequence of SEQ ID NO:17 and a light chain variable region comprising a vlCDR1, a vlCDR2, and a vlCDR3 from a light chain variable region comprising an amino acid sequence of SEQ ID NO:21;
d) a heavy chain variable region comprising a vhCDR1, a vhCDR2 and a vhCDR3 from a heavy chain variable region comprising an amino acid sequence of SEQ ID NO:25 and a light chain variable region comprising a vlCDR1, a vlCDR2, and a vlCDR3 from a light chain variable region comprising an amino acid sequence of SEQ ID NO:29;
e) a heavy chain variable region comprising a vhCDR1, a vhCDR2 and a vhCDR3 from a heavy chain variable region comprising an amino acid sequence of SEQ ID NO:33 a light chain variable region comprising a vlCDR1, a vlCDR2, and a vlCDR3 from a light chain variable region comprising an amino acid sequence of SEQ ID NO:37;
f) a heavy chain variable region comprising a vhCDR1, a vhCDR2 and a vhCDR3 from a heavy chain variable region comprising an amino acid sequence of SEQ ID NO:41 and a light chain variable region comprising a vlCDR1, a vlCDR2, and a vlCDR3 from a light chain variable region comprising an amino acid sequence of SEQ ID NO:45;
g) a heavy chain variable region comprising a vhCDR1, a vhCDR2 and a vhCDR3 from a heavy chain variable region comprising an amino acid sequence of SEQ ID NO:49 and a light chain variable region comprising a vlCDR1, a vlCDR2, and a vlCDR3 from a light chain variable region comprising an amino acid sequence of SEQ ID NO:53;
h) a heavy chain variable region comprising a vhCDR1, a vhCDR2 and a vhCDR3 from a heavy chain variable region comprising an amino acid sequence of SEQ ID NO:57 and a light chain variable region comprising a vlCDR1, a vlCDR2, and a vlCDR3 from a light chain variable region comprising an amino acid sequence of SEQ ID NO:61;
i) a heavy chain variable region comprising a vhCDR1, a vhCDR2 and a vhCDR3 from a heavy chain variable region comprising an amino acid sequence of SEQ ID NO:65 and a light chain variable region comprising a vlCDR1, a vlCDR2, and a vlCDR3 from a light chain variable region comprising an amino acid sequence of SEQ ID NO:69;
j) a heavy chain variable region comprising a vhCDR1, a vhCDR2 and a vhCDR3 from a heavy chain variable region comprising an amino acid sequence of SEQ ID NO:73 and a light chain variable region comprising a vlCDR1, a vlCDR2, and a vlCDR3 from a light chain variable region comprising an amino acid sequence of SEQ ID NO:77;
k) a heavy chain variable region comprising a vhCDR1, a vhCDR2 and a vhCDR3 from a heavy chain variable region comprising an amino acid sequence of SEQ ID NO:81 and a light chain variable region comprising a vlCDR1, a vlCDR2, and a vlCDR3 from a light chain variable region comprising an amino acid sequence of SEQ ID NO:85;
l) a heavy chain variable region comprising a vhCDR1, a vhCDR2 and a vhCDR3 from a heavy chain variable region comprising an amino acid sequence of SEQ ID NO:89 and a light chain variable region comprising a vlCDR1, a vlCDR2, and a vlCDR3 from a light chain variable region comprising an amino acid sequence of SEQ ID NO:93;
m) a heavy chain variable region comprising a vhCDR1, a vhCDR2 and a vhCDR3 from a heavy chain variable region comprising an amino acid sequence of SEQ ID NO:97 and a light chain variable region comprising a vlCDR1, a vlCDR2, and a vlCDR3 from a light chain variable region comprising an amino acid sequence of SEQ ID NO:101;
n) a heavy chain variable region comprising a vhCDR1, a vhCDR2 and a vhCDR3 from a heavy chain variable region comprising an amino acid sequence of SEQ ID NO:105 and a light chain variable region comprising a vlCDR1, a vlCDR2, and a vlCDR3 from a light chain variable region comprising an amino acid sequence of SEQ ID NO:109;
o) a heavy chain variable region comprising a vhCDR1, a vhCDR2 and a vhCDR3 from a heavy chain variable region comprising an amino acid sequence of SEQ ID NO:113 and a light chain variable region comprising a vlCDR1, a vlCDR2, and a vlCDR3 from a light chain variable region comprising an amino acid sequence of SEQ ID NO:117;
p) a heavy chain variable region comprising a vhCDR1, a vhCDR2 and a vhCDR3 from a heavy chain variable region comprising an amino acid sequence of SEQ ID NO:121 and a light chain variable region comprising a vlCDR1, a vlCDR2, and a vlCDR3 from a light chain variable region comprising an amino acid sequence of SEQ ID NO:125;
q) a heavy chain variable region comprising a vhCDR1, a vhCDR2 and a vhCDR3 from a heavy chain variable region comprising an amino acid sequence of SEQ ID NO:129 and a light chain variable region comprising a vlCDR1, a vlCDR2, and a vlCDR3 from a light chain variable region comprising an amino acid sequence of SEQ ID NO:133;
r) a heavy chain variable region comprising a vhCDR1, a vhCDR2 and a vhCDR3 from a heavy chain variable region comprising an amino acid sequence of SEQ ID NO:137 and a light chain variable region comprising a vlCDR1, a vlCDR2, and a vlCDR3 from a light chain variable region comprising an amino acid sequence of SEQ ID NO:141;
s) a heavy chain variable region comprising a vhCDR1, a vhCDR2 and a vhCDR3 from a heavy chain variable region comprising an amino acid sequence of SEQ ID NO:145 and a light chain variable region comprising a vlCDR1, a vlCDR2, and a vlCDR3 from a light chain variable region comprising an amino acid sequence of SEQ ID NO:149;
t) a heavy chain variable region comprising a vhCDR1, a vhCDR2 and a vhCDR3 from a heavy chain variable region comprising an amino acid sequence of SEQ ID NO:153 and a light chain variable region comprising a vlCDR1, a vlCDR2, and a vlCDR3 from a light chain variable region comprising an amino acid sequence of SEQ ID NO:157;
u) a heavy chain variable region comprising a vhCDR1, a vhCDR2 and a vhCDR3 from a heavy chain variable region comprising an amino acid sequence of SEQ ID NO:161 and a light chain variable region comprising a vlCDR1, a vlCDR2, and a vlCDR3 from a light chain variable region comprising an amino acid sequence of SEQ ID NO:165;
v) a heavy chain variable region comprising a vhCDR1, a vhCDR2 and a vhCDR3 from a heavy chain variable region comprising an amino acid sequence of SEQ ID NO:169 and a light chain variable region comprising a vlCDR1, a vlCDR2, and a vlCDR3 from a light chain variable region comprising an amino acid sequence of SEQ ID NO:173;
w) a heavy chain variable region comprising a vhCDR1, a vhCDR2 and a vhCDR3 from a heavy chain variable region comprising an amino acid sequence of SEQ ID NO:177 and a light chain variable region comprising a vlCDR1, a vlCDR2, and a vlCDR3 from a light chain variable region comprising an amino acid sequence of SEQ ID NO:181;
x) a heavy chain variable region comprising a vhCDR1, a vhCDR2 and a vhCDR3 from a heavy chain variable region comprising an amino acid sequence of SEQ ID NO:185 and a light chain variable region comprising a vlCDR1, a vlCDR2, and a vlCDR3 from a light chain variable region comprising an amino acid sequence of SEQ ID NO:189;
y) a heavy chain variable region comprising a vhCDR1, a vhCDR2 and a vhCDR3 from a heavy chain variable region comprising an amino acid sequence of SEQ ID NO:193 and a light chain variable region comprising a vlCDR1, a vlCDR2, and a vlCDR3 from a light chain variable region comprising an amino acid sequence of SEQ ID NO:197;
z) a heavy chain variable region comprising a vhCDR1, a vhCDR2 and a vhCDR3 from a heavy chain variable region comprising an amino acid sequence of SEQ ID NO:201 and a light chain variable region comprising a vlCDR1, a vlCDR2, and a vlCDR3 from a light chain variable region comprising an amino acid sequence of SEQ ID NO:204; or
aa) a heavy chain variable region comprising a vhCDR1, a vhCDR2 and a vhCDR3 from a heavy chain variable region comprising an amino acid sequence of SEQ ID NO:208 and a light chain variable region comprising a vlCDR1, a vlCDR2, and a vlCDR3 from a light chain variable region comprising an amino acid sequence of SEQ ID NO:212.

2. An antibody that binds human LILRB1 or LILRB2, the antibody comprising:
a) a vhCDR1 comprising SEQ ID NO:2, a vhCDR2 comprising SEQ ID NO:3, a vhCDR3 comprising SEQ ID NO:4, a vlCDR1 comprising SEQ ID NO:6, a vlCDR2 comprising SEQ ID NO:7, and a vlCDR3 comprising SEQ ID NO:8;
b) a vhCDR1 comprising SEQ ID NO:10, a vhCDR2 comprising SEQ ID NO:11, a vhCDR3 comprising SEQ ID NO:12, a vlCDR1 comprising SEQ ID NO: 14, a vlCDR2 comprising SEQ ID NO:15, and a vlCDR3 comprising SEQ ID NO: 16;
c) a vhCDR1 comprising SEQ ID NO:18, a vhCDR2 comprising SEQ ID NO:19, a vhCDR3 comprising SEQ ID NO:20, a vlCDR1 comprising SEQ ID NO:22, a vlCDR2 comprising SEQ ID NO:23, and a vlCDR3 comprising SEQ ID NO:24;
d) a vhCDR1 comprising SEQ ID NO:26, a vhCDR2 comprising SEQ ID NO:27, a vhCDR3 comprising SEQ ID NO:28, a vlCDR1 comprising SEQ ID NO:30, a vlCDR2 comprising SEQ ID NO:31, and a vlCDR3 comprising SEQ ID NO: 32;
e) a vhCDR1 comprising SEQ ID NO:34, a vhCDR2 comprising SEQ ID NO:35, a vhCDR3 comprising SEQ ID NO:36, a vlCDR1 comprising SEQ ID NO:38, a vlCDR2 comprising SEQ ID NO:39, and a vlCDR3 comprising SEQ ID NO:40;
f) a vhCDR1 comprising SEQ ID NO:42, a vhCDR2 comprising SEQ ID NO:43, a vhCDR3 comprising SEQ ID NO:44, a vlCDR1 comprising SEQ ID NO:46, a vlCDR2 comprising SEQ ID NO:47, and a vlCDR3 comprising SEQ ID NO:48;
g) a vhCDR1 comprising SEQ ID NO:50, a vhCDR2 comprising SEQ ID NO:51, a vhCDR3 comprising SEQ ID NO:52, a vlCDR1 comprising SEQ ID NO:54, a vlCDR2 comprising SEQ ID NO:55, and a vlCDR3 comprising SEQ ID NO:56;
h) a vhCDR1 comprising SEQ ID NO:58, a vhCDR2 comprising SEQ ID NO:59, a vhCDR3 comprising SEQ ID NO:60, a vlCDR1 comprising SEQ ID NO:62, a vlCDR2 comprising SEQ ID NO:63, and a vlCDR3 comprising SEQ ID NO: 64;
i) a vhCDR1 comprising SEQ ID NO:66, a vhCDR2 comprising SEQ ID NO:67, a vhCDR3 comprising SEQ ID NO:68, a vlCDR1 comprising SEQ ID NO:70, a vlCDR2 comprising SEQ ID NO:71, and a vlCDR3 comprising SEQ ID NO: 72;
j) a vhCDR1 comprising SEQ ID NO:74, a vhCDR2 comprising SEQ ID NO:75, a vhCDR3 comprising SEQ ID NO:76, a vlCDR1 comprising SEQ ID NO:78, a vlCDR2 comprising SEQ ID NO:79, and a vlCDR3 comprising SEQ ID NO: 80;
k) a vhCDR1 comprising SEQ ID NO:82, a vhCDR2 comprising SEQ ID NO:83, a vhCDR3 comprising SEQ ID NO:84, a vlCDR1 comprising SEQ ID NO: 86, a vlCDR2 comprising SEQ ID NO:87, and a vlCDR3 comprising SEQ ID NO: 88;
l) a vhCDR1 comprising SEQ ID NO:90, a vhCDR2 comprising SEQ ID NO:91, a vhCDR3 comprising SEQ ID NO:92, a vlCDR1 comprising SEQ ID NO:94, a vlCDR2 comprising SEQ ID NO:95, and a vlCDR3 comprising SEQ ID NO: 96;
m) a vhCDR1 comprising SEQ ID NO:98, a vhCDR2 comprising SEQ ID NO:99, a vhCDR3 comprising SEQ ID NO:100, a vlCDR1 comprising SEQ ID NO:102, a vlCDR2 comprising SEQ ID NO:103, and a vlCDR3 comprising SEQ ID NO: 104;
n) a vhCDR1 comprising SEQ ID NO:106, a vhCDR2 comprising SEQ ID NO:107, a vhCDR3 comprising SEQ ID NO:108, a vlCDR1 comprising SEQ ID NO:110, a vlCDR2 comprising SEQ ID NO:111, and a vlCDR3 comprising SEQ ID NO:112;
o) a vhCDR1 comprising SEQ ID NO:114, a vhCDR2 comprising SEQ ID NO:115, a vhCDR3 comprising SEQ ID NO:116, a vlCDR1 comprising SEQ ID NO:118, a vlCDR2 comprising SEQ ID NO:119, and a vlCDR3 comprising SEQ ID NO:120;
p) a vhCDR1 comprising SEQ ID NO:122, a vhCDR2 comprising SEQ ID NO:123, a vhCDR3 comprising SEQ ID NO:124, a vlCDR1 comprising SEQ ID NO:126, a vlCDR2 comprising SEQ ID NO:127, and a vlCDR3 comprising SEQ ID NO:128;
q) a vhCDR1 comprising SEQ ID NO:130, a vhCDR2 comprising SEQ ID NO:131, a vhCDR3 comprising SEQ ID NO:132, a vlCDR1 comprising SEQ ID NO:134, a vlCDR2 comprising SEQ ID NO:135, and a vlCDR3 comprising SEQ ID NO:136;
r) a vhCDR1 comprising SEQ ID NO:138, a vhCDR2 comprising SEQ ID NO:139, a vhCDR3 comprising SEQ ID NO:140, a vlCDR1 comprising SEQ ID NO: 142, a vlCDR2 comprising SEQ ID NO:143, and a vlCDR3 comprising SEQ ID NO:144;
s) a vhCDR1 comprising SEQ ID NO:146, a vhCDR2 comprising SEQ ID NO:147, a vhCDR3 comprising SEQ ID NO:148, a vlCDR1 comprising SEQ ID NO:150, a vlCDR2 comprising SEQ ID NO:151, and a vlCDR3 comprising SEQ ID NO:152;
t) a vhCDR1 comprising SEQ ID NO:154, a vhCDR2 comprising SEQ ID NO: 155, a vhCDR3 comprising SEQ ID NO:156, a vlCDR1 comprising SEQ ID NO:158, a vlCDR2 comprising SEQ ID NO:159, and a vlCDR3 comprising SEQ ID NO: 160;
u) a vhCDR1 comprising SEQ ID NO:162, a vhCDR2 comprising SEQ ID NO:163, a vhCDR3 comprising SEQ ID NO:164, a vlCDR1 comprising SEQ ID NO:166, a vlCDR2 comprising SEQ ID NO:167, and a vlCDR3 comprising SEQ ID NO:168;
v) a vhCDR1 comprising SEQ ID NO:170, a vhCDR2 comprising SEQ ID NO:171, a vhCDR3 comprising SEQ ID NO:172, a vlCDR1 comprising SEQ ID NO:174, a vlCDR2 comprising SEQ ID NO:175, and a vlCDR3 comprising SEQ ID NO:176;
w) a vhCDR1 comprising SEQ ID NO:178, a vhCDR2 comprising SEQ ID NO: 179, a vhCDR3 comprising SEQ ID NO:180, a vlCDR1 comprising SEQ ID NO:182, a vlCDR2 comprising SEQ ID NO:183, and a vlCDR3 comprising SEQ ID NO:184;
x) a vhCDR1 comprising SEQ ID NO:186, a vhCDR2 comprising SEQ ID NO:187, a vhCDR3 comprising SEQ ID NO:188, a vlCDR1 comprising SEQ ID NO:190, a vlCDR2 comprising SEQ ID NO:191, and a vlCDR3 comprising SEQ ID NO: 192;
y) a vhCDR1 comprising SEQ ID NO:194, a vhCDR2 comprising SEQ ID NO:195, a vhCDR3 comprising SEQ ID NO:196, a vlCDR1 comprising SEQ ID NO:198, a vlCDR2 comprising SEQ ID NO:199, and a vlCDR3 comprising SEQ ID NO:200;
z) a vhCDR1 comprising SEQ ID NO:202, a vhCDR2 comprising SEQ ID NO:203, a vlCDR1 comprising SEQ ID NO:205, a vlCDR2 comprising SEQ ID NO:206, and a vlCDR3 comprising SEQ ID NO:207; or
aa) a vhCDR1 comprising SEQ ID NO:209, a vhCDR2 comprising SEQ ID NO:210, a vhCDR3 comprising SEQ ID NO:211, a vlCDR1 comprising SEQ ID NO:213, a vlCDR2 comprising SEQ ID NO:214, and a vlCDR3 comprising SEQ ID NO:215.

3. An antibody that binds human LILRB1 or LILRB2, the antibody comprising:
a) a heavy chain variable region comprising an amino acid sequence of SEQ ID NO:1 and a light chain variable region comprising an amino acid sequence of SEQ ID NO: 5;
b) a heavy chain variable region comprising an amino acid sequence of SEQ ID NO:9 and a light chain variable region comprising an amino acid sequence of SEQ ID NO: 13;
c) a heavy chain variable region comprising an amino acid sequence of SEQ ID NO: 17 and a light chain variable region comprising an amino acid sequence of SEQ ID NO:21;
d) a heavy chain variable region comprising an amino acid sequence of SEQ ID NO:25 and a light chain variable region comprising an amino acid sequence of SEQ ID NO:29;
e) a heavy chain variable region comprising an amino acid sequence of SEQ ID NO: 33 a light chain variable region comprising an amino acid sequence of SEQ ID NO: 37;
f) a heavy chain variable region comprising an amino acid sequence of SEQ ID NO:41 and a light chain variable region comprising an amino acid sequence of SEQ ID NO:45;
g) a heavy chain variable region comprising an amino acid sequence of SEQ ID NO:49 and a light chain variable region comprising an amino acid sequence of SEQ ID NO:53;
h) a heavy chain variable region comprising an amino acid sequence of SEQ ID NO:57 and a light chain variable region comprising an amino acid sequence of SEQ ID NO:61;
i) a heavy chain variable region comprising an amino acid sequence of SEQ ID NO: 65 and a light chain variable region comprising an amino acid sequence of SEQ ID NO:69;
j) a heavy chain variable region comprising an amino acid sequence of SEQ ID NO: 73 and a light chain variable region comprising an amino acid sequence of SEQ ID NO:77;
k) a heavy chain variable region comprising an amino acid sequence of SEQ ID NO: 81 and a light chain variable region comprising an amino acid sequence of SEQ ID NO: 85;
l) a heavy chain variable region comprising an amino acid sequence of SEQ ID NO: 89 and a light chain variable region comprising an amino acid sequence of SEQ ID NO:93;
m) a heavy chain variable region comprising an amino acid sequence of SEQ ID NO: 97 and a light chain variable region comprising an amino acid sequence of SEQ ID NO: 101;
n) a heavy chain variable region comprising an amino acid sequence of SEQ ID NO: 105 and a light chain variable region comprising an amino acid sequence of SEQ ID NO: 109;
o) a heavy chain variable region comprising an amino acid sequence of SEQ ID NO: 113 and a light chain variable region comprising an amino acid sequence of SEQ ID NO: 117;
p) a heavy chain variable region comprising an amino acid sequence of SEQ ID NO: 121 and a light chain variable region comprising an amino acid sequence of SEQ ID NO: 125;
q) a heavy chain variable region comprising an amino acid sequence of SEQ ID NO: 129 and a light chain variable region comprising an amino acid sequence of SEQ ID NO: 133;
r) a heavy chain variable region comprising an amino acid sequence of SEQ ID NO: 137 and a light chain variable region comprising an amino acid sequence of SEQ ID NO: 141;
s) a heavy chain variable region comprising an amino acid sequence of SEQ ID NO:145 and a light chain variable region comprising an amino acid sequence of SEQ ID NO: 149;
t) a heavy chain variable region comprising an amino acid sequence of SEQ ID NO: 153 and a light chain variable region comprising an amino acid sequence of SEQ ID NO: 157;
u) a heavy chain variable region comprising an amino acid sequence of SEQ ID NO: 161 and a light chain variable region comprising an amino acid sequence of SEQ ID NO: 165;
v) a heavy chain variable region comprising an amino acid sequence of SEQ ID NO: 169 and a light chain variable region comprising an amino acid sequence of SEQ ID NO: 173;
w) a heavy chain variable region comprising an amino acid sequence of SEQ ID NO: 177 and a light chain variable region comprising an amino acid sequence of SEQ ID NO:181;
x) a heavy chain variable region comprising an amino acid sequence of SEQ ID NO: 185 and a light chain variable region comprising an amino acid sequence of SEQ ID NO:189;
y) a heavy chain variable region comprising an amino acid sequence of SEQ ID NO: 193 and a light chain variable region comprising an amino acid sequence of SEQ ID NO:197;
z) a heavy chain variable region comprising an amino acid sequence of SEQ ID NO: 201 and a light chain variable region comprising an amino acid sequence of SEQ ID NO:204; or
aa) a heavy chain variable region comprising an amino acid sequence of SEQ ID NO:208 and a light chain variable region comprising an amino acid sequence of SEQ ID NO:212.

4. The antibody according to any one of the previous claims, wherein the antibody comprises a constant region with an amino acid sequence at least 90% identical to a human IgG.

5. The antibody according to claim 4, wherein the human IgG is selected from a group consisting of IgG1, IgG2, IgG3 and IgG4.

6. The antibody according to claim 5, wherein the IgG is an IgG1.

7. The antibody according to claim 5, wherein the IgG is an IgG2.

8. A nucleic acid composition encoding the antibody according to any of the previous claims.

9. An expression vector composition comprising the nucleic acid composition according to claim 8, wherein the first nucleic acid is contained in a first expression vector and the second nucleic acid is contained in a second expression vector.

10. An expression vector composition comprising the nucleic acid composition according to claim 8, wherein the first nucleic acid and the second nucleic acid are contained in a single expression vector.

11. A host cell comprising the expression vector composition of claim 9 or 10.

12. A method of making an antibody comprising culturing said host cell of claim 11 under conditions wherein the antibody is expressed, and recovering the antibody.

13. A composition comprising the antibody according to any one of claims 1-7, and a pharmaceutical acceptable carrier or diluent.

14. A method of modulating an immune response in a subject, the method comprising administering to the subject an effective amount of the antibody according to any one of the claims 1-7 or the composition according to claim 13.

15. A method of treating cancer in a subject comprising administering to the subject an effective amount of the antibody according to any one of the claims 1-7 or the composition according to claim 13.

16. The method of claim 15, wherein the cancer upregulates LILRB1 or LILRB2.

17. The method according to any one of the claims 15-16, wherein the antibody is combined with one or more additional therapeutic agents to treat cancer.

18. The method of claim 17, wherein the additional therapeutic agents are other immune checkpoint inhibitors.

19. The method of claim 18, wherein the other immune checkpoint inhibitors are selected from the group consisting of Ipilimumab, Nivolumab, Pembrolizumab, Avelumab, Durvalumab, and Atezolizumab.

20. A method of treating an autoimmune disease in a subject comprising administering to the subject an effective amount of the antibody according to any one of the claims 1-7 or the composition according to claim 13.

21. A method according to claim 20, wherein the antibody is combined with one or more additional therapeutic agents to treat autoimmune disease.

22. A method of treating allergic inflammation in a subject comprising administering to the subject an effective amount of the antibody according to any one of the claims 1-7 or the composition according to claim 13.

23. A method according to claim 22, wherein the antibody is combined with one or more additional therapeutics to treat allergic inflammation.

24. Use of an antibody as described in any of the preceding claims according to a method as described in any of the preceding claims.


SEQUENCE LISTING (215 sequences)

SEQ ID NO: 1 - protein, 299 aa, Artificial Sequence
  peptide
MGWSYIILFLVATATGVHSQVQLQQPGAELVKPGASVKLSCKASGYTFTNYWVQWVKQRPGQGLEWIGMIHPNSGSTNSNEKFKNKATLTVDRSSSTAYMQLSRLTSEDSAVYYCARGDDGYLYYFDYWGQGTTLTVSSAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDQ

SEQ ID NO: 2 - protein, 8 aa, Artificial Sequence
  peptide
GYTFTNYW

SEQ ID NO: 3 - protein, 8 aa, Artificial Sequence
  peptide
IHPNSGST

SEQ ID NO: 4 - protein, 2 aa, Artificial Sequence
  peptide
AR

SEQ ID NO: 5 - protein, 225 aa, Artificial Sequence
  peptide
MDFQVQIFSFLLMSASVIMSRGQIVLTQSPALMSASPGEKVTMTCSASSSVSHMYWYQQKPRSSPKPWIYLTSNLASGVPARFSGSGSGTSYSLTISNMEAEDAATYYCQQWSSYAFTFGSGTKLEIKRADAAPTVSIFPPSSEQLTSGGASVVCFLNNFYPRDINVKWKIDGSERQNGVLNSWTDQDSKDSTYSMSSTLTLTKDEYERHNSYTCEATHKTSTSP

SEQ ID NO: 6 - protein, 5 aa, Artificial Sequence
  peptide
SSVSH

SEQ ID NO: 7 - protein, 3 aa, Artificial Sequence
  peptide
LTS

SEQ ID NO: 8 - protein, 5 aa, Artificial Sequence
  peptide
QQWSS

SEQ ID NO: 9 - protein, 292 aa, Artificial Sequence
  peptide
MEWTWVFLFLLSVTAGVHSQVQLQQSGAELMKPGASVKLSCKATGYTFTGYWIEWVKQRPGHGLEWIGEILLGSGNTNYNENFKGKATLTADTSSNTAYMQLSSLTTEDSAIYYCASTHDGYWGQGTTLTVSSAKTTAPSVYPLAPVCGGTTGSSVTLGCLVKGYFPEPVTLTWNSGSLSSGVHTFPALLQSGLYTLSSSVTVTSNTWPSQTITCNVAHPASSTKVDKKIEPRVPITQNPCPPLKECPPCAAPDLLGGPSVFIFPPKIKDVLMISLSPMVTCVVVDVSEDDQ

SEQ ID NO: 10 - protein, 8 aa, Artificial Sequence
  peptide
GYTFTGYW

SEQ ID NO: 11 - protein, 8 aa, Artificial Sequence
  peptide
ILLGSGNT

SEQ ID NO: 12 - protein, 1 aa, Artificial Sequence
  peptide
A

SEQ ID NO: 13 - protein, 225 aa, Artificial Sequence
  peptide
MHFQVQIFSFLLISASVIMSRGQFILTQSPAIMSASPGEKVTITCSASSSVSYMHWFQQKPGTSPKLWIYSTSNLASGVPARFSGSGSGTSYSLTISRMEAEDAATYYCQRRSSYPFTFGSGTKLEIKRADAAPTVSIFPPSSEQLTSGGASVVCFLNNFYPRDINVKWKIDGSERQNGVLNSWTDQDSKDSTYSMSSTLTLTKDEYERHNSYTCEATHKTSTSP

SEQ ID NO: 14 - protein, 5 aa, Artificial Sequence
  peptide
SSVSY

SEQ ID NO: 15 - protein, 3 aa, Artificial Sequence
  peptide
STS

SEQ ID NO: 16 - protein, 7 aa, Artificial Sequence
  peptide
QRRSSYP

SEQ ID NO: 17 - protein, 287 aa, Artificial Sequence
  peptide
MGWSYIILFLVATATGVHSQVQLQQPGAALVKPGASVKLSCKASGYTFTSYWMHWVKQRPGQGLEWIGMIHPNSGSTNYNEKFRSEATLTVDKSSSTVYMQLSSLTSEDSAVYYCARSGDAYYDGLDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDQ

SEQ ID NO: 18 - protein, 8 aa, Artificial Sequence
  peptide
GYTFTSYW

SEQ ID NO: 19 - protein, 8 aa, Artificial Sequence
  peptide
IHPNSGST

SEQ ID NO: 20 - protein, 2 aa, Artificial Sequence
  peptide
AR

SEQ ID NO: 21 - protein, 224 aa, Artificial Sequence
  peptide
MRVLAELLGLLLFCFLGVRCDIQMNQSPSSLSASLGDTITITCHARQNINVWLSWYQQKPGNIPKLLIYKASNLHTGVSSRFSGSGSGTGFTLTISSLQPEDIATYYCQQGQSYPWTFGGGTKLEIKRADAAPTVSIFPPSSEQLTSGGASVVCFLNNFYPRDINVKWKIDGSERQNGVLNSWTDQDSKDSTYSMSSTLTLTKDEYERHNSYTCEATHKTSTSP

SEQ ID NO: 22 - protein, 6 aa, Artificial Sequence
  peptide
QNINVW

SEQ ID NO: 23 - protein, 3 aa, Artificial Sequence
  peptide
KAS

SEQ ID NO: 24 - protein, 7 aa, Artificial Sequence
  peptide
QQGQSYP

SEQ ID NO: 25 - protein, 287 aa, Artificial Sequence
  peptide
MGWSYIILSLVATATGVHSQVQLQQPGAELVKPGASVKLSCKASGYTFTNYWMYWVKQRPGQGLEWIGMIHPNSDSTNYNEKFKNKATLTVDRSSSTAYMQLSSLTSEDSAVYCCARGEDNYDYVMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDQ

SEQ ID NO: 26 - protein, 8 aa, Artificial Sequence
  peptide
GYTFTNYW

SEQ ID NO: 27 - protein, 8 aa, Artificial Sequence
  peptide
MIHPNSDS

SEQ ID NO: 28 - protein, 2 aa, Artificial Sequence
  peptide
AR

SEQ ID NO: 29 - protein, 229 aa, Artificial Sequence
  peptide
MRCLAEFLGLLVLWIPGAIGDIVMTQAAPSVPVTPGESVSISCRSSKSLLHSYGNTHLYWFLQRPGQSPQLLIYRMSNLASGVPDRFSGSGSGTAFTLRISRVEAEDVGVYYCMQHLEYPLTFGAGTKLELKRADAAPTVSIFPPSSEQLTSGGASVVCFLNNFYPRDINVKWKIDGSERQNGVLNSWTDQDSKDSTYSMSSTLTLTKDEYERHNSYTCEATHKTSTSP

SEQ ID NO: 30 - protein, 12 aa, Artificial Sequence
  peptide
SKSLLHSYGNTH

SEQ ID NO: 31 - protein, 3 aa, Artificial Sequence
  peptide
RMS

SEQ ID NO: 32 - protein, 7 aa, Artificial Sequence
  peptide
MQHLEYP

SEQ ID NO: 33 - protein, 289 aa, Artificial Sequence
  peptide
MGWSWIFFFLLSGTAGVHCQVQLKQSGAELVRPGTSVKLSCKASGYTFTDYYINWVKQRPGQGLEWIARIYPGSGNTFYNEKFQDKATLTAEKSSSTAYMQLSSLTSEDSAVYFCANTPSYGSSHWYFDVWGTGTTVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDQ

SEQ ID NO: 34 - protein, 8 aa, Artificial Sequence
  peptide
GYTFTDYY

SEQ ID NO: 35 - protein, 8 aa, Artificial Sequence
  peptide
IYPGSGNT

SEQ ID NO: 36 - protein, 1 aa, Artificial Sequence
  peptide
A

SEQ ID NO: 37 - protein, 228 aa, Artificial Sequence
  peptide
MGIKMETHSQVFVYMLLWLSGVEGDIVMTQSHKFMSTSVGDRVSITCKASQDVNTAVAWYQQKPGQSPKLLIYWASTRHTGVPDRFTGSGSGTDFTLTISNVQSEDLADYFCQQYSTYPRTFGGGTKLEIKRADAAPTVSIFPPSSEQLTSGGASVVCFLNNFYPRDINVKWKIDGSERQNGVLNSWTDQDSKDSTYSMSSTLTLTKDEYERHNSYTCEATHKTSTSP

SEQ ID NO: 38 - protein, 6 aa, Artificial Sequence
  peptide
QDVNTA

SEQ ID NO: 39 - protein, 3 aa, Artificial Sequence
  peptide
WAS

SEQ ID NO: 40 - protein, 7 aa, Artificial Sequence
  peptide
QQYSTYP

SEQ ID NO: 41 - protein, 284 aa, Artificial Sequence
  peptide
MGWSCIILFLVATATGVHSQVQLQQPGAELVRPGSSVKLSCKASGYTFTSYWMDWVKQRPGQGLEWIGNIYPSDSATHYNQKFKDKATLTVDKSSSTAYMHLSSLTSEDSAVYYCARGLLQYFDVWGTGTSVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDQ

SEQ ID NO: 42 - protein, 8 aa, Artificial Sequence
  peptide
GYTFTSYW

SEQ ID NO: 43 - protein, 8 aa, Artificial Sequence
  peptide
IYPSDSAT

SEQ ID NO: 44 - protein, 2 aa, Artificial Sequence
  peptide
AR

SEQ ID NO: 45 - protein, 224 aa, Artificial Sequence
  peptide
MSVPTQVLGLLLLWLTDARCDIQMTQSPASLSVSVGETVTITCRASENIYSNLAWYQQKQGKSPQLLVYGATNLADGVPSRFSGSGSGTQYSLKINSLQSEDFGSYYCQHFWGIPWTFGGGTKLEIKRADAAPTVSIFPPSSEQLTSGGASVVCFLNNFYPRDINVKWKIDGSERQNGVLNSWTDQDSKDSTYSMSSTLTLTKDEYERHNSYTCEATHKTSTSP

SEQ ID NO: 46 - protein, 6 aa, Artificial Sequence
  peptide
ENIYSN

SEQ ID NO: 47 - protein, 3 aa, Artificial Sequence
  peptide
GAT

SEQ ID NO: 48 - protein, 6 aa, Artificial Sequence
  peptide
HFWGIP

SEQ ID NO: 49 - protein, 289 aa, Artificial Sequence
  peptide
MGWSWIFLFLLSGTAGVHCQIQLQQSGPELVKPGASVKISCKASGYTFTDYYINWVKQRPGQGLEWIGWIYPGGDNTKYNEKFKDKATLTVDTSSSTTYMQLSSLTSEDSAVYFCARFEDKYSSNYWFAYWGQGTLVTVSAAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDQ

SEQ ID NO: 50 - protein, 8 aa, Artificial Sequence
  peptide
GYTFTDYY

SEQ ID NO: 51 - protein, 7 aa, Artificial Sequence
  peptide
YPGGDNT

SEQ ID NO: 52 - protein, 2 aa, Artificial Sequence
  peptide
AR

SEQ ID NO: 53 - protein, 224 aa, Artificial Sequence
  peptide
MVSTPQFLVFLLFWIPASRGDILLTQSPAILSVSPGERVSFSCRASQYIGTSIHWYQQRTNGSPRLLIKYASESISGIPSRFSGSGSGTDFTLSINSVESEDVADYYCQQSNRWPFTFGSGTKLEIKRADAAPTVSIFPPSSEQLTSGGASVVCFLNNFYPRDINVKWKIDGSERQNGVLNSWTDQDSKDSTYSMSSTLTLTKDEYERHNSYTCEATHKTSTSP

SEQ ID NO: 54 - protein, 6 aa, Artificial Sequence
  peptide
QYIGTS

SEQ ID NO: 55 - protein, 3 aa, Artificial Sequence
  peptide
YAS

SEQ ID NO: 56 - protein, 7 aa, Artificial Sequence
  peptide
QQSNRWP

SEQ ID NO: 57 - protein, 285 aa, Artificial Sequence
  peptide
MAVLVLFLCLVAFPNCVLSQVQLKESGPGLVAPSQSLSITCTVSGFSLTSYGIDWVRQPPGKGLEWLGVIWGGGNTNYNSALMSRLTISKDNSKSQVFLKMNSLQTDDTAMYYCAKANWDSYAMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDQ

SEQ ID NO: 58 - protein, 8 aa, Artificial Sequence
  peptide
GFSLTSYG

SEQ ID NO: 59 - protein, 7 aa, Artificial Sequence
  peptide
IWGGGNT

SEQ ID NO: 60 - protein, 2 aa, Artificial Sequence
  peptide
AK

SEQ ID NO: 61 - protein, 224 aa, Artificial Sequence
  peptide
MRVLAELLGLLLFCFLGVRCDIQMNQSPSSLSASLGDTITITCHASQNINFWLNWYQQKPGNIPKLLIYKASNLHTGVPSRFSGSGSGTGFTLTISSLQPEDIATYYCQQGQSYPLTFGGGTKVEIKRADAAPTVSIFPPSSEQLTSGGASVVCFLNNFYPRDINVKWKIDGSERQNGVLNSWTDQDSKDSTYSMSSTLTLTKDEYERHNSYTCEATHKTSTSP

SEQ ID NO: 62 - protein, 7 aa, Artificial Sequence
  peptide
QNINFWL

SEQ ID NO: 63 - protein, 3 aa, Artificial Sequence
  peptide
KAS

SEQ ID NO: 64 - protein, 7 aa, Artificial Sequence
  peptide
QQGQSYP

SEQ ID NO: 65 - protein, 283 aa, Artificial Sequence
  peptide
MEWSWVSLFFLSVTTGVHSQVQLQQSAAELVKPGASVKISCKVSGQSFTDHTIHWMKQRPEQGLEWIGYIFPRDGFTEYNERFKGKATLTADKSSSTAYMHLNSLTSEDSAVYFCAGNWDALDYWGPGTSVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDQ

SEQ ID NO: 66 - protein, 7 aa, Artificial Sequence
  peptide
QSFTDHT

SEQ ID NO: 67 - protein, 8 aa, Artificial Sequence
  peptide
IFPRDGFT

SEQ ID NO: 68 - protein, 1 aa, Artificial Sequence
  peptide
A

SEQ ID NO: 69 - protein, 224 aa, Artificial Sequence
  peptide
MVFTPQILGLMLFWISASRGDIVLTQSPATLSVTPGDSVSLSCRASQSISKYLHWYQQKSHESPRLLIKYVSQSISGIPSRFSGSGSGTDFTLSINSVETEDFGIYFCQQSNSWPLTFGAGTKLELKRADAAPTVSIFPPSSEQLTSGGASVVCFLNNFYPRDINVKWKIDGSERQNGVLNSWTDQDSKDSTYSMSSTLTLTKDEYERHNSYTCEATHKTSTSP

SEQ ID NO: 70 - protein, 6 aa, Artificial Sequence
  peptide
QSISKY

SEQ ID NO: 71 - protein, 3 aa, Artificial Sequence
  peptide
YVS

SEQ ID NO: 72 - protein, 7 aa, Artificial Sequence
  peptide
QQSNSWP

SEQ ID NO: 73 - protein, 285 aa, Artificial Sequence
  peptide
MAVLALLLCLVTFPSCVLSQVQLKESGPGLVAPSQSLSITCTVSGFSLSSYGVNWVRQPPGKGLEWLGVIWGDGSTNYHSALISRLSISKDNSKSQVFLKLNSLQTDDTATYYCAKPNWDYYAMEYWGQGTSVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDQ

SEQ ID NO: 74 - protein, 8 aa, Artificial Sequence
  peptide
GFSLSSYG

SEQ ID NO: 75 - protein, 7 aa, Artificial Sequence
  peptide
IWGDGST

SEQ ID NO: 76 - protein, 2 aa, Artificial Sequence
  peptide
AK

SEQ ID NO: 77 - protein, 224 aa, Artificial Sequence
  peptide
MMSSAQFLGLLLLCFQGTRCDIQMTQTTSSLSASLGDRVTITCRASQDISNFLNWYQQKPDGPVKLLIYYTSRLHSGVPSRFSGSGSGTDYSLTISNLEQEDIATYFCQQGNTLPWTFGGGTKLEIKRADAAPTVSIFPPSSEQLTSGGASVVCFLNNFYPRDINVKWKIDGSERQNGVLNSWTDQDSKDSTYSMSSTLTLTKDEYERHNSYTCEATHKTSTSP

SEQ ID NO: 78 - protein, 6 aa, Artificial Sequence
  peptide
QDISNF

SEQ ID NO: 79 - protein, 3 aa, Artificial Sequence
  peptide
YTS

SEQ ID NO: 80 - protein, 7 aa, Artificial Sequence
  peptide
QQGNTLP

SEQ ID NO: 81 - protein, 285 aa, Artificial Sequence; identical to SEQ ID NO: 73
  peptide

SEQ ID NO: 82 - protein, 9 aa, Artificial Sequence
  peptide
SGFSLSSYG

SEQ ID NO: 83 - protein, 7 aa, Artificial Sequence
  peptide
IWGDGST

SEQ ID NO: 84 - protein, 2 aa, Artificial Sequence
  peptide
AK

SEQ ID NO: 85 - protein, 228 aa, Artificial Sequence
  peptide
MGFKMEFHTQVFVFVFLWLSGVDGGIVMTQSQKFMSSTVGDRVSITCKASQNVGAAVIWYQQKPGQSPKLLIYSASNRYTGVLDRFTGSGSGTDFTLTISNMQSEDLADYFCQQYSSYPWTFGGGTKLEIKRADAAPTVSIFPPSSEQLTSGGASVVCFLNNFYPRDINVKWKIDGSERQNGVLNSWTDQDSKDSTYSMSSTLTLTKDEYERHNSYTCEATHKTSTSP

SEQ ID NO: 86 - protein, 6 aa, Artificial Sequence
  peptide
QNVGAA

SEQ ID NO: 87 - protein, 3 aa, Artificial Sequence
  peptide
SAS

SEQ ID NO: 88 - protein, 7 aa, Artificial Sequence
  peptide
QQYSSYP

SEQ ID NO: 89 - protein, 300 aa, Artificial Sequence
  peptide
MGWSWIFLFLLSGTAGVHCQVQLQQSGPELVKPGASVKISCKASGYSFTSSYIHWVKQRPGQGLEWIGWIYPGSGNTKYNERFKGKATLTADTSSSTAYMHLSSLTSEDSAVYYCARNYGTSYGWYFDVWGTGTTVTVSSAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDQ

SEQ ID NO: 90 - protein, 10 aa, Artificial Sequence
  peptide
ASGYSFTSSY

SEQ ID NO: 91 - protein, 8 aa, Artificial Sequence
  peptide
IYPGSGNT

SEQ ID NO: 92 - protein, 2 aa, Artificial Sequence
  peptide
AR

SEQ ID NO: 93 - protein, 228 aa, Artificial Sequence
  peptide
MGFKMEFHTQVFVFVFLWLSGVDGDIVMTQSQKFMSTTVGDRVSITCKASQNVGTAVAWYQQKPGQSPKLLIYSLSNRYTGVPDRFTGSGSGTDFTLTISNMQSEDLTDYFCQQYSSYPLTFGAGTKLELKRADAAPTVSIFPPSSEQLTSGGASVVCFLNNFYPRDINVKWKIDGSERQNGVLNSWTDQDSKDSTYSMSSTLTLTKDEYERHNSYTCEATHKTSTSP

SEQ ID NO: 94 - protein, 6 aa, Artificial Sequence
  peptide
QNVGTA

SEQ ID NO: 95 - protein, 3 aa, Artificial Sequence
  peptide
SLS

SEQ ID NO: 96 - protein, 7 aa, Artificial Sequence
  peptide
QQYSSYP

SEQ ID NO: 97 - protein, 289 aa, Artificial Sequence
  peptide
MGWSWIFLFLLSGTAGVHCQIQLQQSGPELVKPGASVKISCKASGYTFTDYYINWVKQRPGQGLEWIGWIYPGSGNTKYNEKFKGKATLTVDTSSSTAYMQLSSLTSEDSAVYFCARFEDKHSSNYWFAYWGQGTLVTVSAAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDQ

SEQ ID NO: 98 - protein, 9 aa, Artificial Sequence
  peptide
SGYTFTDYY

SEQ ID NO: 99 - protein, 8 aa, Artificial Sequence
  peptide
IYPGSGNT

SEQ ID NO: 100 - protein, 2 aa, Artificial Sequence
  peptide
AR

SEQ ID NO: 101 - protein, 223 aa, Artificial Sequence
  peptide
MVSTPQFLVFLLFWIPASRGDILLTQSPAILSVSPGERVSFSCRASQYIGTSIHWYQQRTNGSPRLLIKYASESISGIPSRFSGSGSGTDFTLSINSVESEDIADYYCQQSNSWPFTFGSGTKLEIKRADAAPTVSIFPPSSEQLTSGGASVVCFLNNFYPRDINVKWKIDGSERQNGVLNSWTDQDSKDSTYSMSSTLTLTKDEYERHNSYTCEATHKTSTS

SEQ ID NO: 102 - protein, 6 aa, Artificial Sequence
  peptide
QYIGTS

SEQ ID NO: 103 - protein, 3 aa, Artificial Sequence
  peptide
YAS

SEQ ID NO: 104 - protein, 7 aa, Artificial Sequence
  peptide
QQSNSWP

SEQ ID NO: 105 - protein, 289 aa, Artificial Sequence
  peptide
MGWLWNLLFLMAAAQSAQAQIQLVQSGPELKKPGETVKISCKASGYTFTNYGLSWVKQAPGKGLKWMGWINTYSGVTTYADDFKGRFAFSLETSASTAYLQINNLKSEDTATYFCARRSYDYSHYYAMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDQ

SEQ ID NO: 106 - protein, 8 aa, Artificial Sequence
  peptide
GYTFTNYG

SEQ ID NO: 107 - protein, 8 aa, Artificial Sequence
  peptide
INTYSGVT

SEQ ID NO: 108 - protein, 2 aa, Artificial Sequence
  peptide
AR

SEQ ID NO: 109 - protein, 226 aa, Artificial Sequence
  peptide
MTMLSLAPLLSLLLLCVSDSRAETTVTQSPASLSVATGEKVTIRCITSTDIDDDINWYQQKPGEPPKFLISEGNTLRPGVPSRFSSSGYGTDFVFTIENTLSEDVADYYCLQSNNMPYTFGGGTKLEIKRADAAPTVSIFPPSSEQLTSGGASVVCFLNNFYPRDINVKWKIDGSERQNGVLNSWTDQDSKDSTYSMSSTLTLTKDEYERHNSYTCEATHKTSTSP

SEQ ID NO: 110 - protein, 6 aa, Artificial Sequence
  peptide
TDIDDD

SEQ ID NO: 111 - protein, 3 aa, Artificial Sequence
  peptide
EGN

SEQ ID NO: 112 - protein, 6 aa, Artificial Sequence
  peptide
QSNNMP

SEQ ID NO: 113 - protein, 287 aa, Artificial Sequence
  peptide
MNFGLSLIFLALILKGVQCEVQLVESGGDLVKPGESLKLSCAASGFSFSSYDMSWVRQTPDKRLEWVTTISSGGDYTYFPDILKGRFTISRDNAKHTLYLQLSSLKSEDTAMYYCARWDSNFPHWYFDVWGTGTTVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDD

SEQ ID NO: 114 - protein, 8 aa, Artificial Sequence
  peptide
GFSFSSYD

SEQ ID NO: 115 - protein, 9 aa, Artificial Sequence
  peptide
TISSGGDYT

SEQ ID NO: 116 - protein, 2 aa, Artificial Sequence
  peptide
AR

SEQ ID NO: 117 - protein, 228 aa, Artificial Sequence
  peptide
METDTLLLWVLLLWVPGSTGDIVLTQSPASLAVSLGQRATISCRASESVDKSGISFLHWYQQKPRQPPKLLIYRASNLESGIPGRFSGSGSRTDFTLSINPVEPDDVATYYCQQSNKDPFTFGGGTKLEIKRADAAPTVSIFPPSSEQLTSGGASVVCFLNNFYPRDINVKWKIDGSERQNGVLNSWTDQDSKDSTYSMSSTLTLTKDEYERHNSYTCEATHKTSTSP

SEQ ID NO: 118 - protein, 10 aa, Artificial Sequence
  peptide
ESVDKSGISF

SEQ ID NO: 119 - protein, 3 aa, Artificial Sequence
  peptide
RAS

SEQ ID NO: 120 - protein, 7 aa, Artificial Sequence
  peptide
QQSNKDP

SEQ ID NO: 121 - protein, 284 aa, Artificial Sequence
  peptide
MNLGLSLIFLVLVLKGVQCEVKLVESGGGLVQPGGSLKLSCAASGFTFSDYYMYWVRQTPEKRLEWVAYISNGGGNTYYPDTVKGRFTISRDNAKNTLYLQMSRLKSEDTAMYYCARQLEDWYFDVWGTGTTVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDD

SEQ ID NO: 122 - protein, 8 aa, Artificial Sequence
  peptide
GFTFSDYY

SEQ ID NO: 123 - protein, 7 aa, Artificial Sequence
  peptide
SNGGGNT

SEQ ID NO: 124 - protein, 2 aa, Artificial Sequence
  peptide
AR

SEQ ID NO: 125 - protein, 228 aa, Artificial Sequence
  peptide
MGIKMESQIQAFVFVFLWLSGVDGDIVMTQSHKFMSTSVGDRVSITCKASQDVSTAVAWSQQKPGQSPKLLVYWASARHTGVPDRFTGSGSGADYTLTISSVQAEDLALYYCQQHFSTPLTFGAGTKLELKRADAAPTVSIFPPSSEQLTSGGASVVCFLNNFYPRDINVKWKIDGSERQNGVLNSWTDQDSKDSTYSMSSTLTLTKDEYERHNSYTCEATHKTSTSP

SEQ ID NO: 126 - protein, 6 aa, Artificial Sequence
  peptide
QDVSTA

SEQ ID NO: 127 - protein, 3 aa, Artificial Sequence
  peptide
WAS

SEQ ID NO: 128 - protein, 8 aa, Artificial Sequence
  peptide
CQQHFSTP

SEQ ID NO: 129 - protein, 287 aa, Artificial Sequence
  peptide
MNFGLSLIFLALILKGVQCEVQLVESGGDLVKPGGSLKLSCAASGFTFSSYDMSWVRQTPDKRLEWVATISSGGSYTFYPDSVKGRFTISRDNAKNTLSLQMSSLKSEDTAIYYCARWDSNYLRWFFDVWGTGTTVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDD

SEQ ID NO: 130 - protein, 8 aa, Artificial Sequence
  peptide
GFTFSSYD

SEQ ID NO: 131 - protein, 8 aa, Artificial Sequence
  peptide
ISSGGSYT

SEQ ID NO: 132 - protein, 2 aa, Artificial Sequence
  peptide
AR

SEQ ID NO: 133 - protein, 228 aa, Artificial Sequence
  peptide
METDTLLLWVLLLWVPGSTGDIVLTQSPASLAVSLGQRATISCRASESVDNNGISFMHWYQQKPGQSPKLLIYRASNLESGIPARFSGSGSRTDFTLTINPVETDDVATYYCQQSNDDPFTFGGGTKLEIKRADAAPTVSIFPPSSEQLTSGGASVVCFLNNFYPRDINVKWKIDGSERQNGVLNSWTDQDSKDSTYSMSSTLTLTKDEYERHNSYTCEATHKTSTSP

SEQ ID NO: 134 - protein, 10 aa, Artificial Sequence
  peptide
ESVDNNGISF

SEQ ID NO: 135 - protein, 3 aa, Artificial Sequence
  peptide
RAS

SEQ ID NO: 136 - protein, 7 aa, Artificial Sequence
  peptide
QQSNDDP

SEQ ID NO: 137 - protein, 284 aa, Artificial Sequence
  peptide
MNFGLSLIFLALILKGVQCEVQLVESGGDLVKPGGSLKLSCAASGFTFSSYDMSWVRQTPDKRLEWVATINSGGSYSYYPDSVKGRFTMSRDNAKNTLYLQMSSLKSEDTAMYYCARPSYGNSFDYWGQGTSLTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDD

SEQ ID NO: 138 - protein, 8 aa, Artificial Sequence
  peptide
GFTFSSYD

SEQ ID NO: 139 - protein, 8 aa, Artificial Sequence
  peptide
INSGGSYS

SEQ ID NO: 140 - protein, 2 aa, Artificial Sequence
  peptide
AR

SEQ ID NO: 141 - protein, 233 aa, Artificial Sequence
  peptide
MHHTSMGIKMESQIQVFVFVFLWLSGVDGDIVMTQSHKFKSTSVGDRVNITCKASQDVSIAVAWYQQKPGQSPKLLIYAASYRYTGVPDRFTGSGSGTDFTFTISSVQAEDLAVYYCQQHYSIPWTFGGGTKLEIKRADAAPTVSIFPPSSEQLTSGGASVVCFLNNFYPRDINVKWKIDGSERQNGVLNSWTDQDSKDSTYSMSSTLTLTKDEYERHNSYTCEATHKTSTSP

SEQ ID NO: 142 - protein, 6 aa, Artificial Sequence
  peptide
QDVSIA

SEQ ID NO: 143 - protein, 3 aa, Artificial Sequence
  peptide
AAS

SEQ ID NO: 144 - protein, 7 aa, Artificial Sequence
  peptide
QQHYSIP

SEQ ID NO: 145 - protein, 294 aa, Artificial Sequence
  peptide
MGWSWIFLFLLSGTAGVLSEVQLQQSGPELVKPGASVKISCKASGYTFTDDYMNWVKQSHGKSLEWIGDINPTNGDTNYNQKFKGKATLTVDKSSSTAYMELRGLTSEDSAVYYCARGSSPFTYWGQGTLVTVSAAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDD

SEQ ID NO: 146 - protein, 8 aa, Artificial Sequence
  peptide
GYTFTDDY

SEQ ID NO: 147 - protein, 8 aa, Artificial Sequence
  peptide
INPTNGDT

SEQ ID NO: 148 - protein, 2 aa, Artificial Sequence
  peptide
AR

SEQ ID NO: 149 - protein, 224 aa, Artificial Sequence
  peptide
MMSSAQFLGLLLLCFQGSRCDIQMTQTTSSLSASLGDRVTISCSASQGISNYLNWYQQKPDGTVKLLIYYTSSLYSGVPSRFSGSGSGTDYSLTISNLEPEDIATYYCQQYSKLPWTFGGGTKLEIKRADAAPTVSIFPPSSEQLTSGGASVVCFLNNFYPRDINVKWKIDGSERQNGVLNSWTDQDSKDSTYSMSSTLTLTKDEYERHNSYTCEATHKTSTSP

SEQ ID NO: 150 - protein, 6 aa, Artificial Sequence
  peptide
QGISNY

SEQ ID NO: 151 - protein, 3 aa, Artificial Sequence
  peptide
YTS

SEQ ID NO: 152 - protein, 7 aa, Artificial Sequence
  peptide
QQYSKLP

SEQ ID NO: 153 - protein, 270 aa, Artificial Sequence
  peptide
MNLGLSLIFLVLVLKGVQCEVKLVESGGGLVQPGGSLKLSCAASGFTFSDYYMYWVRQTPEKRLEWVAYISNGGGNTYYPDTVKGRFTISRDNAKNTLYLQMSRLKSEDTAMYYCARNRDDWYFDVWGTGTTVTVSSAKTTAPSVYPLAPVCGGTTGSSVTLGCLVKGYFPEPVTLTWNSGSLSSGVHTFPALLQSGLYTLSSSVTVTSNTWPSQTITCNVAHPASSTKVDKKIEPRVPITQNPCPPLKECPPCAAPDLLGGPSVFIFPP

SEQ ID NO: 154 - protein, 8 aa, Artificial Sequence
  peptide
GFTFSDYY

SEQ ID NO: 155 - protein, 7 aa, Artificial Sequence
  peptide
SNGGGNT

SEQ ID NO: 156 - protein, 2 aa, Artificial Sequence
  peptide
AR

SEQ ID NO: 157 - protein, 230 aa, Artificial Sequence
  peptide
MESQTQVLMFLLLWVSGACADIVMTQSPSSLAMSVGQKVTMNCKSSQSLLISSNQKNYLAWYQQKPGQSPKLLIYFASTRESGVPDRFIGSGSGTDFTLTISSVQAEDLADYFCQQHYSTPLTFGAGTKLELKRADAAPTVSIFPPSSEQLTSGGASVVCFLNNFYPRDINVKWKIDGSERQNGVLNSWTDQDSKDSTYSMSSTLTLTKDEYERHNSYTCEATHKTSTSP

SEQ ID NO: 158 - protein, 12 aa, Artificial Sequence
  peptide
QSLLISSNQKNY

SEQ ID NO: 159 - protein, 3 aa, Artificial Sequence
  peptide
FAS

SEQ ID NO: 160 - protein, 7 aa, Artificial Sequence
  peptide
QQHYSTP

SEQ ID NO: 161 - protein, 287 aa, Artificial Sequence
  peptide
MGWSCIILILVAAATGVHSQVQLQQPGAELVKPGASVKMSCKASDYTFTSYWITWVKKRPGQGLEWIGDIYPGSDTTNYNEKFKNKATLTVDTSSSTAHMQLSSLTSEDSAVYYCTRPLYQGISPWFAYWGQGTLVTVSAAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDD

SEQ ID NO: 162 - protein, 8 aa, Artificial Sequence
  peptide
DYTFTSYW

SEQ ID NO: 163 - protein, 8 aa, Artificial Sequence
  peptide
IYPGSDTT

SEQ ID NO: 164 - protein, 2 aa, Artificial Sequence
  peptide
TR

SEQ ID NO: 165 - protein, 233 aa, Artificial Sequence
  peptide
MHQTSMGIKMESQTLVFISILLWLYGADGNIVMTQSPKSMSMSVGERVTLSCKASENVGTYVSWYQQKPEQSPKLLIYGASNRYTGVPDRFTGSGSATDFTLTISSVQAEDLADYHCGQSYSYPFTFGSGTKLEIKRADAAPTVSIFPPSSEQLTSGGASVVCFLNNFYPRDINVKWKIDGSERQNGVLNSWTDQDSKDSTYSMSSTLTLTKDEYERHNSYTCEATHKTSTSP

SEQ ID NO: 166 - protein, 6 aa, Artificial Sequence
  peptide
ENVGTY

SEQ ID NO: 167 - protein, 3 aa, Artificial Sequence
  peptide
GAS

SEQ ID NO: 168 - protein, 7 aa, Artificial Sequence
  peptide
GQSYSYP

SEQ ID NO: 169 - protein, 182 aa, Artificial Sequence
  peptide
MGWSWIFLFLLSGTAGVLSEVQLQESGPELLKPGASMKISCKASGYTFTDDYMNWVKQSHGKSLEWIGDINPNNGGTSYNQKFKGKATLTVDKSSSTAYMELRSLTSEDSAVYYCARGSPWFAYWGQGTLVTVSAAKTTPPSVYPLAPGSAAQTNSMVTLGCLVKGYFPEPVTVTWNSGSLS

SEQ ID NO: 170 - protein, 8 aa, Artificial Sequence
  peptide
GYTFTDDY

SEQ ID NO: 171 - protein, 8 aa, Artificial Sequence
  peptide
INPNNGGT

SEQ ID NO: 172 - protein, 2 aa, Artificial Sequence
  peptide
AR

SEQ ID NO: 173 - protein, 224 aa, Artificial Sequence
  peptide
MMSSAQFLGLLLLCFQGTRCDIQMTQTTSSLSASLGDRVTINCRASQDIRNYLSWYQQKPDGTVKLLIYYTSRLHSGVPSRFSGSGSGRDYSLTISNLEQEDIATYFCQQSNTLPWTFGGGTKLEITRADAAPTVSIFPPSSEQLTSGGASVVCFLNNFYPRDINVKWKIDGSERQNGVLNSWTDQDSKDSTYSMSSTLTLTKDEYERHNSYTCEATHKTSTSP

SEQ ID NO: 174 - protein, 6 aa, Artificial Sequence
  peptide
QDIRNY

SEQ ID NO: 175 - protein, 3 aa, Artificial Sequence
  peptide
YTS

SEQ ID NO: 176 - protein, 7 aa, Artificial Sequence
  peptide
QQSNTLP

SEQ ID NO: 177 - protein, 288 aa, Artificial Sequence
  peptide
MGWLWNLLFLMAAAQSAQAQIQLVQSGPELKKPGETVKISCKASGYTFTTYGMSWVKQAPGKGLKWMGWINTYSGAPAYVDDFKGRFAFSLETSASTAYLQINNLKNEDTATYFCARHFYSGSSYWYFDVWGTGTTVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDD

SEQ ID NO: 178 - protein, 8 aa, Artificial Sequence
  peptide
GYTFTTYG

SEQ ID NO: 179 - protein, 7 aa, Artificial Sequence
  peptide
NTYSGAP

SEQ ID NO: 180 - protein, 2 aa, Artificial Sequence
  peptide
AR

SEQ ID NO: 181 - protein, 224 aa, Artificial Sequence
  peptide
MSVPTQVLGLLLLWLTAARCDIQMTQSPASLSVSVGETVTITCRASENIYSNLAWYQQKQGKSPQLLVYAATNLADGVPSRFSGSGSGTQYSLKINSLQSEDFGSYYCQHFWGTPRTFGGGTKLEIKRADAAPTVSIFPPSSEQLTSGGASVVCFLNNFYPRDINVKWKIDGSERQNGVLNSWTDQDSKDSTYSMSSTLTLTKDEYERHNSYTCEATHKTSTSP

SEQ ID NO: 182 - protein, 6 aa, Artificial Sequence
  peptide
ENIYSN

SEQ ID NO: 183 - protein, 3 aa, Artificial Sequence
  peptide
AAT

SEQ ID NO: 184 - protein, 7 aa, Artificial Sequence
  peptide
QHFWGTP

SEQ ID NO: 185 - protein, 284 aa, Artificial Sequence
  peptide
MNFGLSLIFLALILKGVQCEVQLVESGGDFVKPGGSLKLSCAASGFTFSSYDMSWVRQTPDKRLEWVATIIRGDSYTYYLDSVKGRFTISRDNAKNTLYLQMSSLKSEDTAMYYCARPSYGNSFDYWGQGTTLTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDD

SEQ ID NO: 186 - protein, 8 aa, Artificial Sequence
  peptide
GFTFSSYD

SEQ ID NO: 187 - protein, 8 aa, Artificial Sequence
  peptide
IIRGDSYT

SEQ ID NO: 188 - protein, 2 aa, Artificial Sequence
  peptide
AR

SEQ ID NO: 189 - protein, 233 aa, Artificial Sequence
  peptide
MHHTSMGIKMESQIQVFVFVFLWLSGVDGDIVMTQSHKFMSTSIGDRVSITCKASQDVNNAVAWYQQKPGQSPKLLIYAASYRYTGVPDRFTGSGSGTDFTFTISSVQAEDLAVYHCQQHYGIPWTFGGGTKLEIKRADAAPTVSIFPPSSEQLTSGGASVVCFLNNFYPRDINVKWKIDGSERQNGVLNSWTDQDSKDSTYSMSSTLTLTKDEYERHNSYTCEATHKTSTSP

SEQ ID NO: 190 - protein, 6 aa, Artificial Sequence
  peptide
QDVNNA

SEQ ID NO: 191 - protein, 3 aa, Artificial Sequence
  peptide
AAS

SEQ ID NO: 192 - protein, 7 aa, Artificial Sequence
  peptide
QQHYGIP

SEQ ID NO: 193 - protein, 282 aa, Artificial Sequence
  peptide
MKVLSLLYLLTAIPGILSDVQLQESGPGLVKPSQSLSLTCSVTGYSITSGYYWNWIRQFPGNKLEWMGYISYDGSNNYNPSLKNRISITRDTSKNQFFLKLNSVTTEDTATYYCARGGWLLSRYWGQGTSVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDD

SEQ ID NO: 194 - protein, 9 aa, Artificial Sequence
  peptide
GYSITSGYY

SEQ ID NO: 195 - protein, 7 aa, Artificial Sequence
  peptide
ISYDGSN

SEQ ID NO: 196 - protein, 2 aa, Artificial Sequence
  peptide
AR

SEQ ID NO: 197 - protein, 224 aa, Artificial Sequence
  peptide
MSVPTQVLGLLLLWLTGARCDIQMTQSPASLSASVGETVTITCRASENIYSYLAWYQQKQGKSPQLLVYNAKTLAEGVPSRFSGSGSDTQFSLKINSLQPEDFGIFYCQHFYGDLPTFGAGTKLELKRADAAPTVSIFPPSSEQLTSGGASVVCFLNNFYPRDINVKWKIDGSERQNGVLNSWTDQDSKDSTYSMSSTLTLTKDEYERHNSYTCEATHKTSTSP

SEQ ID NO: 198 - protein, 6 aa, Artificial Sequence
  peptide
ENIYSY

SEQ ID NO: 199 - protein, 3 aa, Artificial Sequence
  peptide
NAK

SEQ ID NO: 200 - protein, 5 aa, Artificial Sequence
  peptide
QHFYG

SEQ ID NO: 201 - protein, 280 aa, Artificial Sequence
  peptide
MEWSWVSLFFLSVTTGVHSQVQLQQSDAELVKPGASVKISCKVSGYTFSDHTFHWMKQRPEQGLEWIGYFYPRDGRSKYNEKFRDKATLTADKSSSTAYMQLNSLTSEDSAVYFCTCDGFDYWGQGTTLTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDD

SEQ ID NO: 202 - protein, 8 aa, Artificial Sequence
  peptide
GYTFSDHT

SEQ ID NO: 203 - protein, 8 aa, Artificial Sequence
  peptide
FYPRDGRS

SEQ ID NO: 204 - protein, 226 aa, Artificial Sequence
  peptide
MDMRTPAQFLGILLLWFPGIKCDIKMTQSPSSMHASLGERVTITCKASQDINSYLSWFQQKPGKSPKTLIYHADRLVDGVPSRFSGSGSGQDYSLTISSLEYEDMGIYYCLQYDEFPYTFGGGTKLEIKRADAAPTVSIFPPSSEQLTSGGASVVCFLNNFYPRDINVKWKIDGSERQNGVLNSWTDQDSKDSTYSMSSTLTLTKDEYERHNSYTCEATHKTSTSP

SEQ ID NO: 205 - protein, 6 aa, Artificial Sequence
  peptide
QDINSY

SEQ ID NO: 206 - protein, 3 aa, Artificial Sequence
  peptide
HAD

SEQ ID NO: 207 - protein, 7 aa, Artificial Sequence
  peptide
LQYDEFP

SEQ ID NO: 208 - protein, 287 aa, Artificial Sequence
  peptide
MNFGLSLIFLALILKGVQCEVQLVESGGDLVKPGGSLKLSCAASGFTFSSYDMSWVRQTPDKRLEWVATISSGASYTYYPDSVKGRFTISRDNAKNTLYLQMSSLKSEDTAIYYCARWDSKYLRWYFDVWGTGTTVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDD

SEQ ID NO: 209 - protein, 8 aa, Artificial Sequence
  peptide
GFTFSSYD

SEQ ID NO: 210 - protein, 9 aa, Artificial Sequence
  peptide
TISSGASYT

SEQ ID NO: 211 - protein, 2 aa, Artificial Sequence
  peptide
AR

SEQ ID NO: 212 - protein, 228 aa, Artificial Sequence
  peptide
METDTLLLWVLLLWVPGSTGDIVLTQSPASLAVSLGQRATISCRASESVDNNGISFMHWYQQKPGQSPKLLISRASNLESGIPARFSGSGSRTDFTLTINPVETDDVATYYCQQSNEDPFTFGGGTKLEIKRADAAPTVSIFPPSSEQLTSGGASVVCFLNNFYPRDINVKWKIDGSERQNGVLNSWTDQDSKDSTYSMSSTLTLTKDEYERHNSYTCEATHKTSTSP

SEQ ID NO: 213 - protein, 10 aa, Artificial Sequence
  peptide
ESVDNNGISF

SEQ ID NO: 214 - protein, 3 aa, Artificial Sequence
  peptide
RAS

SEQ ID NO: 215 - protein, 7 aa, Artificial Sequence
  peptide
QQSNEDP